(19) United States
(12) Patent Application Publication
(10) Pub. No.: US 2004/0127539 A1
(43) Pub. Date: Jul. 1, 2004

(54) Antiangiogenic combination therapy for the treatment of cancer

(75) Inventor: Jaime L. Masferrer, Ballwin, MO (US)

Correspondence Address: Charles E. Dunlap, P.O. Box 11070, Columbia, SC, 29211-1070, US

(73) Assignee: Pharmacia Corporation, St. Louis, MO

(21) Appl. No.: 10/692,643

(22) Filed: Oct. 24, 2003

Related U.S. Application Data
(62) Division of application No. 09/843,132, filed on Apr. 25, 2001.
Application No. 09/843,132 is a continuation-in-part of application No. 09/470,951, filed on Dec. 22, 1999, now abandoned.
(60) Provisional application No. 60/113,786, filed on Dec. 23, 1998.

(51) Int. Cl. A61K 31/365; A61K 31/415

(52) U.S. Cl. 514/406; 514/473

(57) ABSTRACT

The present invention provides combinations of a DNA topoisomerase I inhibiting agent and a selective Cox-2 inhibiting agent for preventing, treating, and/or reducing the risk of developing a neoplasia disorder in a mammal.

### CROSS-REFERENCE TO RELATED PATENT APPLICATIONS

This application is a divisional of U.S. patent application Ser. No. 09/843,132, filed Apr. 25, 2001, which is a continuation-in-part of U.S. patent application Ser. No. 09/470,951, filed Dec. 22, 1999, which is a non-provisional of U.S. patent application Serial No. 60/113,786, filed Dec. 23, 1998, and claims the priority benefit of these three applications, each of which is incorporated herein by reference in its entirety.

### BACKGROUND OF THE INVENTION

(1) Field of the Invention

The present invention relates to methods, combinations and compositions for treating, preventing or reducing the risk of developing a neoplasia disorder in a mammal.

(2) Description of Related Art

Cancer is now the second leading cause of death in the United States. In 1995 over 8,000,000 persons in the United States have been diagnosed with cancer and has accounted for 23.3% of all reported deaths.

Cancer is not fully understood on the molecular level. It is known that exposure of a cell to a carcinogen such as certain viruses, certain chemicals, or radiation, leads to DNA alteration that inactivates a “suppressive” gene or activates an “oncogene.” Suppressive genes are growth regulatory genes, which upon mutation, can no longer control cell growth. Oncogenes are initially normal genes (called prooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A neoplasm, or tumor, is an abnormal, unregulated, and disorganized proliferation of cell growth, and is generally referred to as cancer. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer today is primarily treated with one or more types of anticancer therapy, including surgery, radiation and chemotherapy. Surgery involves the bulk removal of diseased tissue. While surgery is sometimes effective in removing tumors located at certain sites, for example, in the breast, colon, or skin, it cannot be used in the treatment of tumors located in other areas, such as the backbone, nor in the treatment of disseminated neoplastic conditions such as leukemia. Radiation therapy involves the exposure of living tissue to ionizing radiation causing death or damage to the exposed cells. Side effects from radiation therapy may be acute and temporary, while others may be irreversible. Chemotherapy involves the disruption of cell replication or cell metabolism. Chemotherapy is used most often in the treatment of breast, lung, and testicular cancer.

The adverse side effects of anticancer therapy is most feared by patients undergoing treatment for cancer. Of these adverse effects pain, nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications, such as pruritis, urticaria, and angioedema; neurological complications; pulmonary and cardiac complications in patients receiving radiation or chemotherapy; and reproductive and endocrine complications. Anticancer therapy induced side effects significantly impact the quality of life of the patient and may dramatically influence patient compliance with treatment.

Additionally, the adverse side effects associated with anticancer therapy is generally the major dose-limiting toxicity (DLT) in the administration of the therapy. For example, mucositis, is one of the major dose limiting toxicity for several anticancer agents, including the antimetabolite cytotoxic agents 5-FU, and methotrexate, and antitumor antibiotics, such as doxorubicin. Many of these chemotherapy-induced side effects if severe, may lead to hospitalization, or require treatment with analgesics for the treatment of pain.

Adverse side effects induced by anticancer therapy have become of major importance in the clinical management of patients undergoing treatment for cancer or neoplasia disease.

### SUMMARY OF THE INVENTION

In brief, the present invention provides a method for treating, preventing or reducing the risk of developing a neoplasia disorder in a mammal in need thereof, comprising administering to the mammal in a combination therapy an amount of a DNA topoisomerase I inhibiting agent and an amount of a selective cyclooxygenase-2 inhibiting agent wherein the amount of the DNA topoisomerase I inhibiting agent and the selective cyclooxygenase-2 inhibiting agent together make a neoplasia disorder effective amount.

The present invention further provides a pharmaceutical composition comprising a DNA topoisomerase I inhibiting agent and a cyclooxygenase-2 inhibiting agent wherein the DNA topoisomerase I inhibiting agent and the selective cyclooxygenase-2 inhibiting agent together make a neoplasia disorder effective amount.

In another embodiment, the present invention provides a use of a composition in preparation of a medicament useful in treating, preventing or lowering the risk of developing a neoplasia disorder in a mammal in need thereof, the composition comprising an amount of a DNA topoisomerase I inhibiting agent and an amount of a cyclooxygenase-2 inhibiting agent wherein the amount of the DNA topoisomerase I inhibiting agent and the selective cyclooxygenase-2 inhibiting agent together make a neoplasia disorder effective amount.

The present invention further provides a kit comprising a DNA topoisomerase I inhibiting agent and a selective cyclooxygenase-2 inhibiting agent wherein the DNA topoisomerase I inhibiting agent and the selective cyclooxygenase-2 inhibiting agent together make a neoplasia disorder effective amount.

Another embodiment of the present invention provides a method for the prevention or treatment of DNA topoisomerase I inhibiting agent-related diarrhea in a subject in need of such prevention or treatment wherein the method comprises administering to the subject a diarrhea preventing or treating-effective amount of a source of a Cox-2 inhibiting agent, thereby preventing or treating the DNA topoisomerase I inhibiting agent-related diarrhea.

### DESCRIPTION OF THE PREFERRED EMBODIMENTS

Definitions

In the written descriptions of molecules and groups, molecular descriptors can be combined to produce words or phrases that describe structural groups or are combined to describe structural groups. Such descriptors are used in this document. Common illustrative examples include such terms as aralkyl (or arylalkyl), heteroaralkyl, heterocycloalkyl, cycloalkylalkyl, aralkoxyalkoxycarbonyl, and the like. A specific example of a compound encompassed with the latter descriptor aralkoxyalkoxycarbonyl is C₆H₅—CH₂—CH₂—O—CH₂—O—(C═O)— wherein C₆H₅— is phenyl. It is also to be noted that a structural group can have more than one descriptive word or phrase in the art, for example, heteroaryloxyalkylcarbonyl can also be termed heteroaryloxyalkanoyl. Such combinations are used herein in the description of the processes, compounds and compositions of this invention and further examples are described below. The following list is not intended to be exhaustive or drawn out but provide illustrative examples of words or phrases (terms) that are used herein.

As utilized herein, the term “alkyl”, alone or in combination, means a straight-chain or branched-chain alkyl radical containing one to about twelve carbon atoms, preferably one to about ten carbon atoms, and more preferably one to about six carbon atoms. Examples of such radicals include methyl, ethyl, n-propyl, isopropyl, n-butyl, isobutyl, sec-butyl, tert-butyl, pentyl, iso-amyl, hexyl, octyl, and the like.

The term “alkenyl”, alone or in combination, means a straight-chain or branched-chain hydrocarbon radical having one or more double bonds and containing two to about twenty carbon atoms preferably two to about twelve carbon atoms, and more preferably, two to about six carbon atoms. Examples of suitable alkenyl radicals include ethenyl (vinyl), 2-propenyl, 3-propenyl, allyl, 1,4-pentadienyl, 1,4-butadienyl, 1-butenyl, 2-butenyl, 3-butenyl, 4-methylbutenyl, decenyl, and the like. The term “alkenyl” embrace radicals having “cis” and “trans” orientations, or alternatively, “E” and “Z” orientations.

The term “alkynyl”, alone or in combination, means a straight-chain or branched-chain hydrocarbon radical having one or more triple bonds and containing two to about twelve carbon atoms, preferably two to about ten carbon atoms, and more preferably, two to about six carbon atoms. Examples of alkynyl radicals include ethynyl, 2-propynyl, 3-propynyl, decynyl, 1-butynyl, 2-butynyl, 3-butynyl, propargyl, and the like.

The term “acyl”, alone or in combination, means a radical provided by the residue after removal of hydroxyl from an organic acid. Examples of such acyl radicals include alkanoyl and aroyl radicals. Examples of such alkanoyl radicals include formyl, acetyl, propionyl, butyryl, isobutyryl, valeryl, isovaleryl, pivaloyl, hexanoyl, trifluoroacetyl, and the like.

The term “carbonyl” or “oxo”, alone or in combination, i.e., used with other terms, such as “alkoxycarbonyl”, means a —C(═O)— group wherein the remaining two bonds (valences) can be independently substituted. The term carbonyl is also intended to encompass a hydrated carbonyl group —C(OH)₂—.

The term “hydrido”, alone or in combination, means a single hydrogen atom (H). This hydrido radical may be attached, for example, to an oxygen atom to form a hydroxyl radical or two hydrido radicals may be attached to a carbon atom to form a methylene (—CH₂—) radical.

The term “halo”, alone or in combination, means halogen such as fluoride, chloride, bromide or iodide.

The term “haloalkyl”, alone or in combination, means an alkyl radical having the significance as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals.

More preferred haloalkoxy radicals are haloalkoxy radicals having one to six carbon atoms and one or more halo radicals. Examples of such haloalkyl radicals include chloromethyl, dichloromethyl, trichloromethyl, 1-bromoethyl, fluoromethyl, difluoromethyl, trifluoromethyl, 1,1,1-trifluoroethyl, pentafluoroethyl, heptafluoropropyl, difluorochloromethyl, dichlorofluoromethyl, difluoroethyl, difluoropropyl, dichloroethyl, dichloropropyl, and the like.

Examples of such radicals include fluoromethoxy, chloromethoxy, trifluoromethoxy, trifluoroethoxy, fluoroethoxy, fluoropropoxy, and the like.

The term “perfluoroalkyl”, alone or in combination, means an alkyl group wherein each hydrogen has been replaced by a fluorine atom. Examples of such perfluoroalkyl groups, in addition to trifluoromethyl above, are perfluorobutyl, perfluoroisopropyl, perfluorododecyl and perfluorodecyl.

The term “perfluoroalkoxy”, alone or in combination, means a perfluoroalkyl ether radical wherein the term perfluoroalkyl is as defined above. Examples of such perfluoroalkoxy groups, in addition to trifluoromethoxy (F₃C—O—), are perfluorobutoxy, perfluoroisopropoxy, perfluorododecoxy and perfluorodecoxy.

The term “perfluoroalkylthio”, alone or in combination, means a perfluoroalkyl thioether radical wherein the term perfluoroalkyl is as defined above. Examples of such perfluoroalkylthio groups, in addition to trifluoromethylthio (F₃C—S—), are perfluorobutylthio, perfluoroisopropylthio, perfluorododecylthio and perfluorodecylthio.

The term “hydroxyalkyl”, alone or in combination, means a linear or branched alkyl radical having one to about ten carbon atoms any one of which may be substituted with one or more hydroxyl radicals. Preferred hydroxyalkyl radicals have one to six carbon atoms and one or more hydroxyl radicals. Examples of such radicals include hydroxymethyl, hydroxyethyl, hydroxypropyl, hydroxybutyl and hydroxyhexyl.

The term “thiol” or “sulfhydryl”, alone or in combination, means a —SH group. The term “thio” or “thia”, alone or in combination, means a thiaether group; i.e., an ether group wherein the ether oxygen is replaced by a sulfur atom.

The term “amino”, alone or in combination, means an amine or —NH₂ group whereas the term mono-substituted amino, alone or in combination, means a substituted amine —N(H)(substituent) group wherein one hydrogen atom is replaced with a substituent, and disubstituted amine means a —N(substituent)₂ wherein two hydrogen atoms of the amino group are replaced with independently selected substituent groups.

Amines, amino groups and amides are compounds that can be designated as primary (I°), secondary (II°) or tertiary (III°) or unsubstituted, mono-substituted or N,N-disubstituted depending on the degree of substitution of the amino nitrogen. Quaternary amine (ammonium)(IV°) means a nitrogen with four substituents [—N⁺(substituent)₄] that is positively charged and accompanied by a counter ion, whereas N-oxide means one substituent is oxygen and the group is represented as [—N⁺(substituent)₃—O⁻]; i.e., the charges are internally compensated.

The term “cyano”, alone or in combination, means a —C-triple bond-N (—C≡N) group.

The term “azido”, alone or in combination, means a —N-triple bond-N (—N≡N) group.

The term “hydroxyl”, alone or in combination, means a —OH group.

The term “nitro”, alone or in combination, means a —NO₂ group.

The term “azo”, alone or in combination, means a —N═N— group wherein the bonds at the terminal positions can be independently substituted.

The term “hydrazino”, alone or in combination, means a —NH—NH— group wherein the depicted remaining two bonds (valences) can be independently substituted. The hydrogen atoms of the hydrazino group can be replaced, independently, with substituents and the nitrogen atoms can form acid addition salts or be quaternized.

The term “sulfonyl”, alone or in combination, i.e., linked to other terms such as alkylsulfonyl, means a —SO₂— group wherein the depicted remaining two bonds (valences) can be independently substituted.

The term “sulfoxido”, alone or in combination, means a —SO— group wherein the remaining two bonds (valences) can be independently substituted.

The term “sulfone”, alone or in combination, means a —SO₂— group wherein the depicted remaining two bonds (valences) can be independently substituted.

The term “sulfenamide”, alone or in combination, means a —SON═ group wherein the remaining three depicted bonds (valences) can be independently substituted.

The term “sulfide”, alone or in combination, means a —S— group wherein the remaining two bonds (valences) can be independently substituted.

The term “alkylthio”, alone or in combination, means a radical containing a linear or branched alkyl radical, of one to about ten carbon atoms attached to a divalent sulfur atom. More preferred alkylthio radicals are radicals having alkyl radicals of one to six carbon atoms. Examples of such alkylthio radicals are methylthio, ethylthio, propylthio, butylthio and hexylthio.

The term “alkylthioalkyl”, alone or in combination, means a radical containing an alkylthio radical attached through the divalent sulfur atom to an alkyl radical of one to about ten carbon atoms. More preferred alkylthioalkyl radicals are radicals having alkyl radicals of one to six carbon atoms. Examples of such alkylthioalkyl radicals include methylthiomethyl, methylthioethyl, ethylthioethyl, and ethylthiomethyl.

The term “alkylsulfinyl”, alone or in combination, means a radical containing a linear or branched alkyl radical, of one to ten carbon atoms, attached to a divalent —S(═O)— radical. More preferred alkylsulfinyl radicals are radicals having alkyl radicals of one to six carbon atoms. Examples of such alkylsulfinyl radicals include methylsulfinyl, ethylsulfinyl, butylsulfinyl and hexylsulfinyl.

The term “alkylsulfonyl”, alone or in combination, means an alkyl radical attached to a sulfonyl radical, where alkyl is defined as above. More preferred alkylsulfonyl radicals are alkylsulfonyl radicals having one to six carbon atoms. Examples of such alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl”, alone or in combination, mean a NH₂O₂S— radical.

The term “alkoxy” or “alkyloxy”, alone or in combination, mean an alkyl ether radical wherein the term alkyl is as defined above. Examples of suitable alkyl ether radicals include methoxy, ethoxy, n-propoxy, isopropoxy, n-butoxy, iso-butoxy, sec-butoxy, tert-butoxy, and the like. The “alkoxy” radicals may be further substituted with one or more halo atoms, such as fluoro, chloro or bromo, to provide haloalkoxy radicals. More preferred haloalkoxy radicals are “haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxyalkyl”, alone or in combination, means an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals.

The term “cycloalkyl”, alone or in combination, means a cyclic alkyl radical that contains three to about twelve carbon atoms. More preferred cycloalkyl radicals are cycloalkyl radicals having three to about eight carbon atoms. Examples of such radicals include cyclopropyl, cyclobutyl, cyclopentyl, cyclohexyl, and the like

The term “cycloalkylalkyl”, alone or in combination, means an alkyl radical as defined above that is substituted by a cycloalkyl radical containing three to about eight, preferably three to about six, carbon atoms. Examples of such cycloalkyl radicals include cyclopropyl, cyclobutyl, cyclopentyl, cyclohexyl, and the like.

The term “cycloalkenyl” means partially unsaturated carbocyclic radicals having three to twelve carbon atoms. More preferred cycloalkenyl radicals are cycloalkenyl radicals having four to about eight carbon atoms. Examples of such radicals include cyclobutenyl, cyclopentenyl, cyclohexenyl, and the like.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated three- to six-membered heteromonocylic group containing one to four nitrogen atoms (e.g. pyrrolidinyl, imidazolidinyl, piperidino, piperazinyl, etc.); saturated three- to six-membered heteromonocyclic group containing one to two oxygen atoms and one to three nitrogen atoms (e.g. morpholinyl, etc.); saturated three- to six-membered heteromonocyclic group containing one to two sulfur atoms and one to three nitrogen atoms (e.g., thiazolidinyl, etc.). Examples of partially unsaturated heterocyclo radicals include dihydrothiophene, dihydropyran, dihydrofuran and dihydrothiazole. A heterocyclic (heterocyclo) portion of a heterocyclocarbonyl, heterocyclooxy-carbonyl, heterocycloalkoxycarbonyl, or heterocycloalkyl group or the like is a saturated or partially unsaturated monocyclic, bicyclic or tricyclic heterocycle that contains one or more hetero atoms selected from nitrogen, oxygen and sulphur. Heterocyclo compounds include benzofused heterocyclic compounds such as benzo-1,4-dioxane. Such a moiety can be optionally substituted on one or more ring carbon atoms by halogen, hydroxy, hydroxycarbonyl, alkyl, alkoxy, oxo, and the like, and/or on a secondary nitrogen atom (i.e., —NH—) of the ring by alkyl, aralkoxycarbonyl, alkanoyl, aryl or arylalkyl or on a tertiary nitrogen atom (i.e., ═N—) by oxido and that is attached via a carbon atom. The tertiary nitrogen atom with three substituents can also attached to form a N-oxide [═N(O)—] group.

The term “heterocycloalkyl”, alone or in combination, means a saturated and partially unsaturated heterocyclo-substituted alkyl radical, such as pyrrolidinylmethyl, and heteroaryl-substituted alkyl, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aryl”, alone or in combination, means a five- or six-membered carbocyclic aromatic ring-containing moiety or a five- or six-membered carbocyclic aromatic system containing two or three rings wherein such rings are attached together in a pendent manner, or a fused ring system containing two or three rings that have all carbon atoms in the ring; i.e., a carbocyclic aryl radical. The term “aryl” embraces aromatic radicals such as phenyl, indenyl, naphthyl, tetrahydronaphthyl, indane and biphenyl. Aryl moieties may also be substituted with one or more substituents including alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heteroaryl”, alone or in combination means a five- or six-membered aromatic ring-containing moiety or a fused ring system (radical) containing two or three rings that have carbon atoms and also one or more heteroatoms in the ring(s) such as sulfur, oxygen and nitrogen. Examples of such heterocyclic or heteroaryl groups are pyrrolidinyl, piperidyl, piperazinyl, morpholinyl, thiamorpholinyl, pyrrolyl, imidazolyl (e.g., imidazol-4-yl, 1-benzyloxycarbonylimidazol-4-yl, and the like), pyrazolyl, pyridyl, pyrazinyl, pyrimidinyl, furyl, tetrahydrofuryl, thienyl, triazolyl, tetrazolyl, oxazolyl, oxadiazoyl, thiazolyl, thiadiazoyl, indolyl (e.g., 2-indolyl, and the like), quinolinyl, (e.g., 2-quinolinyl, 3-quinolinyl, 1-oxido-2-quinolinyl, and the like), isoquinolinyl (e.g., 1-isoquinolinyl, 3-isoquinolinyl, and the like), tetrahydroquinolinyl (e.g., 1,2,3,4-tetrahydro-2-quinolyl, and the like), 1,2,3,4-tetrahydroisoquinolinyl (e.g., 1,2,3,4-tetrahydro-1-oxo-isoquinolinyl, and the like), quinoxalinyl, β-carbolinyl, 2-benzofurancarbonyl, benzothiophenyl, 1-, 2-, 4- or 5-benzimidazolyl, and the like radicals.

The term “aralkyl”, alone or in combination, means an alkyl radical as defined above in which one hydrogen atom is replaced by an aryl radical as defined above, such as benzyl, diphenylmethyl, triphenylmethyl, phenylethyl, diphenylethyl 2-phenylethyl, and the like. The aryl in said aralkyl may be additionally substituted with halo, alkyl, alkoxy, halkoalkyl and haloalkoxy. The terms benzyl and phenylmethyl are interchangeable.

The term “aralkoxy”, alone or in combination, means an aralkyl radical attached through an oxygen atom to other radicals.

The term “aralkoxyalkyl”, alone or in combination, means an aralkoxy radical attached through an oxygen atom to an alkyl radical.

The term “aralkylthio”, alone or in combination, means an aralkyl radical attached to a sulfur atom.

The term “aralkylthioalkyl”, alone or in combination, means an aralkylthio radical attached through a sulfur atom to an alkyl radical.

The term “aralkoxycarbonyl”, alone or in combination, means a radical of the formula aralkyl-O—C(O)— in which the term “aralkyl” has the significance given above. An example of an aralkoxycarbonyl radical is benzyloxycarbonyl.

The term “aryloxy”, alone or in combination, means a radical of the formula aryl-O— in which the term aryl has the significance given above. The phenoxy radical is an exemplary aryloxy radical.

The term “aminoalkyl”, alone or in combination, means an alkyl radical substituted with amino radicals. Preferred are aminoalkyl radicals having alkyl portions having one to six carbon atoms. Examples of such radicals include aminomethyl, aminoethyl, and the like.

The term “alkylamino”, alone or in combination, means an amino group which has been substituted with one or two alkyl radicals. Preferred are N-alkylamino radicals having alkyl portions having one to six carbon atoms. Suitable alkylamino may be mono or dialkylamino such as N-methylamino, N-ethylamino, N,N-dimethylamino, N,N-diethylamino, and the like.

The term “arylamino”, alone or in combination, means an amino group which has been substituted with one or two aryl radicals, such as N-phenylamino. The “arylamino” radicals may be further substituted on the aryl ring portion of the radical.

The term “aralkylamino”, alone or in combination, means an aralkyl radical attached through a nitrogen atom to other radicals. The terms “N-arylaminoalkyl” and “N-aryl-N-alkyl-aminoalkyl” mean an amino group which have been substituted with one aryl radical or one aryl and one alkyl radical, respectively, and having the amino group attached to an alkyl radical. Examples of such radicals include N-phenylaminomethyl, N-phenyl-N-methylaminomethyl, and the like.

The terms “heteroaralkyl” and “heteroaryloxy”, alone or in combination, mean a radical structurally similar to aralkyl and aryloxy that are formed from heteroaryl radicals. Exemplary radicals include 4-picolinyl and 2-pyrimidinoxy, respectively.

The terms “alkanoyl” or “alkylcarbonyl”, alone or in combination, mean an acyl radical derived from an alkanecarboxylic acid, examples of which include formyl, acetyl, propionyl, butyryl, valeryl, 4-methylvaleryl, and the like.

The term “cycloalkylcarbonyl”, alone or in combination, means an acyl group derived from a monocyclic or bridged cycloalkanecarboxylic acid such as cyclopropanecarbonyl, cyclohexanecarbonyl, adamantanecarbonyl, and the like, or from a benz-fused monocyclic cycloalkanecarboxylic acid that is optionally substituted by, for example, alkanoylamino, such as 1,2,3,4-tetrahydro-2-naphthoyl, 2-acetamido-1,2,3,4-tetrahydro-2-naphthoyl.

The terms “aralkanoyl” or “aralkylcarbonyl”, alone or in combination, mean an acyl radical derived from an aryl-substituted alkanecarboxylic acid such as phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The terms “aroyl” or “arylcarbonyl”, alone or in combination, mean an acyl radical derived from an aromatic carboxylic acid. Examples of such radicals include aromatic carboxylic acids, an optionally substituted benzoic or naphthoic acid such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The terms “carboxy” or “carboxyl”, whether used alone or in combination, i.e., with other terms, such as “carboxyalkyl”, mean a —CO₂H radical.

The term “carboxyalkyl”, alone or in combination, means an alkyl radical substituted with a carboxy radical. More preferred carboxyalkyl radicals have alkyl radicals as defined above, and may be additionally substituted on the alkyl radical with halo. Examples of such carboxyalkyl radicals include carboxymethyl, carboxyethyl, carboxypropyl, and the like.

The term “alkoxycarbonyl”, alone or in combination, means a radical containing an alkoxy radical, as defined above, attached via an oxygen atom to a carbonyl radical. More preferred alkoxycarbonyl radicals have alkyl portions having one to six carbons. Examples of such alkoxycarbonyl (ester) radicals include substituted or unsubstituted methoxycarbonyl, ethoxycarbonyl, propoxycarbonyl, butoxycarbonyl, hexyloxycarbonyl, and the like.

The term “cycloalkylalkoxycarbonyl”, alone or in combination, means an acyl group of the formula cycloalkylalkyl-O—CO— wherein cycloalkylalkyl has the significance given above.

The term “aryloxyalkanoyl”, alone or in combination, means an acyl radical of the formula aryl-O-alkanoyl wherein aryl and alkanoyl have the significance given above.

The term “heterocyclooxycarbonyl”, alone or in combination, means an acyl group having the formula heterocyclo-O—CO— wherein heterocyclo is as defined above.

The term “heterocycloalkanoyl”, alone or in combination, means an acyl radical of the formula heterocyclo-substituted alkane carboxylic acid wherein heterocyclo has the significance given above.

The term “heterocycloalkoxycarbonyl”, alone or in combination, means an acyl radical of the formula heterocyclo-substituted alkane-O—CO— wherein heterocyclo has the significance given above.

The term “heteroaryloxycarbonyl”, alone or in combination, means an acyl radical represented by the formula heteroaryl-O—CO— wherein heteroaryl has the significance given above.

The term “aminocarbonyl” (carboxamide) alone or in combination, means an amino-substituted carbonyl (carbamoyl) group derived from an amine reacted with a carboxylic acid wherein the amino (amido nitrogen) group is unsubstituted (—NH₂) or a substituted primary or secondary amino group containing one or more substituents selected from hydrogen, alkyl, aryl, aralkyl, cycloalkyl, cycloalkylalkyl, and the like, as recited. A hydroxamate is a N-hydroxycarboxamide.

The term “alkylaminoalkyl”, alone or in combination, means a radical having one or more alkyl radicals attached to an aminoalkyl radical.

The term “aryloxyalkyl”, alone or in combination, means a radical having an aryl radical attached to an alkyl radical through a divalent oxygen atom.

The term “arylthioalkyl”, alone or in combination, means a radical having an aryl radical attached to an alkyl radical through a divalent sulfur atom.

The term “aminoalkanoyl”, alone or in combination, means an acyl group derived from an amino-substituted alkanecarboxylic acid wherein the amino group can be a primary or secondary amino group containing substituents independently selected from hydrogen, alkyl, aryl, aralkyl, cycloalkyl, cycloalkylalkyl, and the like.

The term “aromatic ring” in combinations such as substituted-aromatic ring sulfone or substituted-aromatic ring sulfoxide means aryl or heteroaryl as defined before.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Combinations and Methods

The present invention provides a method for treating, preventing or reducing the risk of developing a neoplasia disorder in a mammal. The method comprises administering to the mammal in a combination therapy an amount of a DNA topoisomerase I inhibiting agent and a cyclooxygenase-2 inhibiting agent, wherein the DNA topoisomerase I inhibiting agent and the cyclooxygenase-2 inhibiting agent together make a neoplasia disorder effective amount. The present invention further provides a method of halting or slowing the progression of neoplastic disease once it becomes clinically evident. Also provided by the present inventive the methods, combinations and compositions of the present invention are pharmaceutical compositions comprising a DNA topoisomerase I inhibiting agent and a cyclooxygenase-2 inhibiting agent where the individual agents together make a neoplasia disorder effective amount. The present invention also provides a kit comprising a cyclooxygenase-2 inhibiting agent and a DNA topoisomerase I inhibiting agent. When administered as part of a combination therapy, the cyclooxygenase-2 inhibiting agent together with the DNA topoisomerase I inhibiting agent provide enhanced treatment options for treating, preventing, and reducing the risk of developing neoplastic disease in a mammal as compared to administration of either a DNA topoisomerase I inhibiting agent or a cyclooxygenase-2 inhibiting agent alone.

The present invention further provides a method for the prevention or treatment of DNA topoisomerase I inhibiting agent-related diarrhea in a subject in need of such prevention or treatment wherein the method comprises administering to the subject a diarrhea preventing or treating-effective amount of a source of a Cox-2 inhibiting agent, thereby preventing or treating the DNA topoisomerase I inhibiting agent-related diarrhea. Preferably, the source of a Cox-2 inhibiting agent is a source of a Cox-2 selective inhibiting agent, and more preferably a Cox-2 selective inhibiting agent. For example, the Cox-2 selective inhibiting agent can be celecoxib, valdecoxib, deracoxib, rofecoxib, etoricoxib, meloxicam, or ABT-963. Alternatively, the Cox-2 selective inhibiting agent can be a chromene Cox-2 selective inhibiting agent. In another embodiment, the source of a Cox-2 selective inhibiting agent can be a prodrug of a Cox-2 selective inhibiting agent. For example, the prodrug can be parecoxib. Preferably the DNA topoisomerase I inhibiting agent is selected from the group consisting of irinotecan; irinotecan hydrochloride; camptothecin; 9-aminocamptothecin; 9-nitrocamptothecin; 9-chloro-10-hydroxy camptothecin; topotecan; lurtotecan; a homosilatecan; 6,8-dibromo-2-methyl-3-[2-(D-xylopyranosylamino)phenyl]-4(3H)-quinazolinone; 2-cyano-3-(3,4-dihydroxyphenyl)-N-(phenylmethyl)-(2E)-2-propenamide; 2-cyano-3-(3,4-dihydroxyphenyl)-N-(3-hydroxyphenylpropyl)-(E)-2-propenamide; 12-beta-D-glucopyranosyl-12,13-dihydro-2,10-dihydroxy-6-[[2-hydroxy-1-(hydroxymethyl)ethyl]amino]-5H-indolo[2,3-a]pyrrolo[3,4-c]carbazole-5,7(6H)-dione; N-[2-(dimethylamino)ethyl]-4-acridinecarboxamide, dihydrochloride; and N-[2-(dimethylamino)ethyl]-4-acridinecarboxamide; or a salt of the DNA topoisomerase I inhibiting agent.

Preferably, the DNA topoisoermerase I inhibiting agent is selected from the group consisting of irinotecan, rubitecan, lurtotecan, exetecan mesylate, karenitecan, and silatecan; or a salt of one of these agents. More preferably still, the DNA topoisomerase I inhibiting agent is irinotecan. When the DNA topoisomerase I inhibiting agent is irinotecan, the source of a Cox-2 inhibiting agent is preferably a source of a Cox-2 selective inhibiting agent, and more preferably selected from the group consisting of celecoxib, valdecoxib, deracoxib, rofecoxib, etoricoxib, meloxicam, and ABT-963. Alternatively, the source of a Cox-2 selective inhibiting agent can be a chromene Cox-2 selective inhibiting agent. In another embodiment, when the DNA topoisomerase I inhibiting agent is irinotecan, the source of a Cox-2 inhibiting agent can be a prodrug of a Cox-2 selective inhibiting agent, preferably parecoxib. For treatment or prevention of the DNA topoisomerase I inhibiting agent-related diarrhea, the source of a Cox-2 selective inhibiting agent can be administered to the subject by essentially any convenient route. For example, the source of a Cox-2 selective inhibiting agent can be administered orally, parenterally (e.g., intravenously, subcutaneously, or intramuscularly), transdermally, or rectally. The source of a Cox-2 inhibiting agent and the DNA topoisomerase I inhibiting agent can be administered to the subject in essentially any convenient regimen. For example, the source of the Cox-2 selective inhibiting agent can be administered to the subject before treating the subject with the DNA topoisomerase I inhibiting agent. Alternatively, the source of the Cox-2 selective inhibiting agent can be administered to the subject concurrently with treating the subject with the DNA topoisomerase I inhibiting agent. In another alternative the source of the Cox-2 selective inhibiting agent can be administered to the subject after treating the subject with the DNA topoisomerase I inhibiting agent.

A source of a Cox-2 inhibiting agent can be, for example, a source of a Cox-2 selective inhibiting agent, or a source of a nonselective cyclooxygenase inhibiting agent. The source of a Cox-2 selective inhibiting agent can be, for example, a Cox-2 selective inhibiting agent or a prodrug of a Cox-2 selective inhibiting agent.

Besides being useful for human treatment, the present invention is also useful for veterinary treatment of companion mammals, exotic animals and farm animals, including mammals, rodents, and the like. In one embodiment, the mammals include horses, dogs, and cats.

There are many uses for the present inventive combination. For example, DNA topoisomerase I inhibiting agents and Cox-2 selective inhibiting agents (or prodrugs thereof) are each believed to be effective antineoplastic or antiangiogenic agents. However, patients treated with a DNA topoisomerase I inhibiting agent frequently experience side effects such as diarrhea. The present inventive combination will allow the subject to be administered a DNA topoisomerase I inhibitor at a therapeutically effective dose yet experience reduced or fewer symptoms of diarrhea. A further use and advantage is that the present inventive combination will allow therapeutically effective individual dose levels of the DNA topoisomerase I inhibitor and the selective cyclooxygenase-2 inhibitor which are lower than the dose levels of each inhibitor when administered to the patient as a monotherapy.

Some therapeutic compounds which are useful in the present inventive combination include compounds which selectively inhibit cyclooxygenase-2 (Cox-2) relative to cyclooxygenase-1 (Cox-1) (i.e., a “Cox-2 selective inhibiting agent”). In one embodiment, the compounds have a selectivity ratio of Cox-2 inhibition relative to Cox-1 inhibition of at least 50, and in another embodiment have a selectivity ratio of at least 100. Inhibitors of the cyclooxygenase pathway in the metabolism of arachidonic acid used in the treatment, prevention or reduction in the risk of developing neoplasia disease may inhibit enzyme activity through a variety of mechanisms. By way of example, the cyclooxygenase inhibitors used in the methods described herein may block the enzyme activity directly by acting as a substrate for the enzyme. The use of a Cox-2 selective inhibiting agent is highly advantageous in that they minimize the gastric side effects that can occur with non-selective non-steroidal antiinflammatory drugs (NSAIDs), especially where prolonged treatment is expected.

A class of Cox-2 selective inhibiting agents useful in the methods, combinations and compositions of the present invention include compounds of Formula 1:

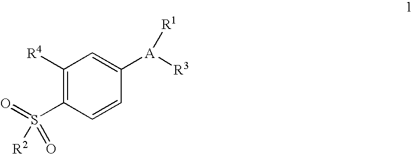

wherein

A is a 5- or 6-member ring substituent selected from aryl, heteroaryl, heterocyclo, and cycloalkyl, wherein A is optionally substituted with one or more radicals selected from hydroxy, alkyl, halo, oxo, and alkoxy;

R¹ is cyclohexyl, pyridinyl, or phenyl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, phenylamino, nitro, alkoxyalkyl, alkylsulfinyl, halo, alkoxy, and alkylthio;

R² is alkyl or amino;

R³ is selected from the group consisting of halo, alkyl, alkenyl, alkynyl, aryl, heteroaryl, oxo, cyano, carboxyl, cyanoalkyl, heterocyclyloxy, alkyloxy, alkylthio, alkylcarbonyl, cycloalkyl, phenyl, haloalkyl, heterocyclo, cycloalkenyl, phenylalkyl, heterocycloalkyl, alkylthioalkyl, hydroxyalkyl, alkoxycarbonyl, phenylcarbonyl, phenylalkylcarbonyl, phenylalkenyl, alkoxyalkyl, phenylthioalkyl, phenyloxyalkyl, alkoxyphenylalkoxyalkyl, alkoxycarbonylalkyl, aminocarbonyl, aminocarbonylalkyl, alkylaminocarbonyl, N-phenylaminocarbonyl, N-alkyl-N-phenylaminocarbonyl, alkylaminocarbonylalkyl, carboxyalkyl, alkylamino, N-arylamino, N-arylkylamino, N-alkyl-N-arylkylamino, N-alkyl-N-arylamino, aminoalkyl, alkylaminoalkyl, N-phenylaminoalkyl, N-phenylalkylaminoalkyl, N-alkyl-N-phenylalkylaminoalkyl, N-alkyl-N-phenylaminoalkyl, phenyloxy, phenylalkoxy, phenylthio, phenylalkylthio, alkylsulfinyl, alkylsulfonyl, aminosulfonyl, alkylaminosulfonyl, N-phenylaminosulfonyl, phenylsulfonyl, and N-alkyl-N-phenylaminosulfonyl; and

R⁴ is hydrido or halo;

or an isomer, tautomer, pharmaceutically-acceptable salt or prodrug thereof.

Within Formula 1 there is a subclass of compounds of particular interest wherein A is thienyl, oxazolyl, furyl, furanone, pyrrolyl, thiazolyl, imidazolyl, benzofuryl, indenyl, benzithienyl, isoxazolyl, pyrazolyl, cyclopentenyl, cyclopentadienyl, benzindazolyl, cyclopentenone, benzopyranopyrazolyl, phenyl, or pyridyl;

R¹ is cyclohexyl, pyridinyl, and phenyl, wherein cyclohexyl, pyridinyl, or phenyl, wherein R¹ is optionally substituted with one or more radicals selected from alkyl, haloalkyl, cyano, carboxyl, alkoxycarbonyl, hydroxyl, hydroxyalkyl, haloalkoxy, amino, alkylamino, phenylamino, nitro, alkoxyalkyl, alkylsulfinyl, alkoxy, halo, alkoxy, and alkylthio;

R² is methyl or amino; and

R³ is halo, alkyl, alkenyl, alkynyl, aryl, heteroaryl, oxo, cyano, carboxyl, cyanoalkyl, heterocyclyloxy, alkyloxy, alkylthio, alkylcarbonyl, cycloalkyl, phenyl, haloalkyl, heterocyclo, cycloalkenyl, phenylalkyl, heterocyclylalkyl, alkylthioalkyl, hydroxyalkyl, alkoxycarbonyl, phenylcarbonyl, phenylalkylcarbonyl, phenylalkenyl, alkoxyalkyl, phenylthioalkyl, phenyloxyalkyl, alkoxyphenylalkoxyalkyl, alkoxycarbonylalkyl, aminocarbonyl, aminocarbonylalkyl, alkylaminocarbonyl, N-phenylaminocarbonyl, N-alkyl-N-phenylaminocarbonyl, alkylaminocarbonyl-alkyl, carboxy-alkyl, alkylamino, N-arylamino, N-arylkylamino, N-alkyl-N-arylkylamino, N-alkyl-N-arylamino, amino-alkyl, alkylaminoalkyl, N-phenylamino-alkyl, N-phenyl-alkylaminoalkyl, N-alkyl-N-phenyl-alkylamino-alkyl, N-alkyl-N-phenylaminoalkyl, phenyloxy, phenylalkoxy, phenylthio, phenylalkylthio, alkylsulfinyl, alkylsulfonyl, aminosulfonyl, alkylaminosulfonyl, N-phenylaminosulfonyl, phenylsulfonyl, or N-alkyl-N-phenylaminosulfonyl;

or an isomer, tautomer, pharmaceutically-acceptable salt or prodrug thereof.

A preferred class of compounds within Formula 1 includes compounds wherein A is substituted with one or more radicals selected alkyl, halo, oxo, and alkoxy;

R¹ is pyridyl, cyclohexyl, or phenyl, wherein R¹ is optionally substituted with one or more radicals selected from alkyl, halo, and alkoxy;

R³ is halo, alkyl, cyano, carboxyl, alkyloxy, phenyl, haloalkyl, or hydroxyalkyl; and

R⁴ is hydrido or fluoro;

or an isomer, tautomer, pharmaceutically-acceptable salt or prodrug thereof.

A family within Formula 1 which are particularly preferred include the following compounds and their pharmaceutically-acceptable salts:

4-(4-(methylsulfonyl)phenyl]-3-phenyl-2(5H)-furanone (rofecoxib),

4-[5-(4-methylphenyl)-3-(trifluoromethyl)-1H-pyrazol-1-yl]-benzenesulfonamide (celecoxib),

4-[5-methyl-3-phenyl-3-phenylisoxazol-4-yl]benzensulfonamide (valdecoxib),

4-[5-(3-fluoro-4mthoxyphenyl)-3-difluoromethyl)-1H-pyrazol-1-yl]bensenesulfonamide (deracoxib),

4-(4-cyclohexyl-2-methyloxazol-5-yl)-2-fluorobenzenesulfonamide (JTE-522),

2-(6-methylpyrid-3-yl)-3-(4-methylsulfinylphenyl)-5-chloropyridine (MK-663),

5-chloro-3-(4-(methylsulfonyl)phenyl)-2-(methyl-5-pyridinyl)pyridine,

2-(3,5-difluorophenyl)-3-4-(methylsulfonyl)phenyl)-2-cyclopenten-1-one,

N-[[4-(5-methyl-3-phenylisoxazol-4yl]phenyl]sulfonyl]propanamide,

4-[5-(4-chorophenyl)-3-(trifluoromethyl)-1H-pyrazole-1-yl]benzenesulfonamide,

3-(3,4-difluorophenoxy)-5,5-dimethyl-4-[4-(methylsulfonyl)phenyl]-2(5H)-furanone,

N-[6-[(2,4-difluorophenyl)thio]-2,3-dihydro-1-oxo-1H-inden-5-yl]methanesulfonamide,

3-(4-chlorophenyl)-4-[4-(methylsulfonyl)phenyl]-2(3H)-oxazolone,

4-[3-(4-fluorophenyl)-2,3-dihydro-2-oxo-4-oxazolyl]benzenesulfonamide,

3-[4-(methylsulfonyl)phenyl]-2-phenyl-2-cyclopenten-1-one,

4-(2-methyl-4-phenyl-5-oxazolyl)benzenesulfonamide,

3-(4-fluorophenyl)-4-[4-(methylsulfonyl)phenyl]-2(3H)-oxazolone,

5-(4-fluorophenyl)-1-[4-(methylsulfonyl)phenyl]-3-(trifluoromethyl)-1H-pyrazole,

4-[5-phenyl)-3-(trifluoromethyl)-1H-pyrazol-1-yl)benzenesulfonamide,

4-[1-phenyl-3-(trifluoromethyl)-1H-pyrazol-5-yl]benzenesulfonamide,

4-[5-(4-fluorophenyl)-3-(trifluoromethyl)-1H-pyrazol-1-yl]benzenesulfonamide,

N-[2-(cyclohexyloxy)-4-nitrophenyl]methanesulfonamide,

N-[6-(2,4-difluorophenoxy)-2,3-dihydro-1-oxo-1H-inden-5-yl]methanesulfonamide,

3-(4-chlorophenoxy)-4-[(methylsulfonyl)amino]benzenesulfonamide,

3-(4-fluorophenoxy)-4-[(methylsulfonyl)amino]benzenesulfonamide,

3-[(1-methyl-1H-imidazol-2-yl)thio]-4 [(methylsulfonyl) amino]benzenesulfonamide,

5,5-dimethyl-4-[4-(methylsulfonyl)phenyl]-3-phenoxy-2(5H)-furanone,

N-[6-[(4-ethyl-2-thiazolyl)thio]-1,3-dihydro-1-oxo-5-isobenzofuranyl]methanesulfonamide,

3-[(2,4-dichlorophenyl)thio]-4-[(methylsulfonyl)amino]benzenesulfonamide,

1-fluoro-4-[2-[4-(methylsulfonyl)phenyl]cyclopenten-1-yl]benzene,

4-[5-(4-chlorophenyl)-3-(difluoromethyl)-1H-pyrazol-1-yl]benzenesulfonamide,

3-[1-[4-(methylsulfonyl)phenyl]-4-(trifluoromethyl)-1H-imidazol-2-yl]pyridine,

4-[2-(3-pyridinyll)-4-(trifluoromethyl)-1H-imidazol-1-yl]benzenesulfonamide,

4-[5-(hydroxymethyl)-3-phenylisoxazol-4-yl]benzenesulfonamide,

4-[3-(4-chlorophenyl)-2,3-dihydro-2-oxo-4-oxazolyl]benzenesulfonamide,

4-[5-(difluoromethyl)-3-phenylisoxazol-4-yl]benzenesulfonamide,

[1,1′:2′,1″-terphenyl]-4-sulfonamide,

4-(methylsulfonyl)-1,1′,2],1″-terphenyl,

4-(2-phenyl-3-pyridinyl)benzenesulfonamide,

N-(2,3-dihydro-1,1-dioxido-6-phenoxy-1,2-benzisothiazol-5-yl)methanesulfonamide,

N-[3-(formylamino)-4-oxo-6-phenoxy-4H-1-benzopyran-7-yl]methanesulfonamide,

6-[[5-(4-chlorobenzoyl)-1,4-dimethyl-1H-pyrrol-2-yl]methyl]-3(2H)-pyridazinone, and

N-(4-nitro-2-phenoxyphenyl)methanesulfonamide.

Specific compounds of particular interest within Formula 1 include each of the compounds and pharmaceutically-acceptable salts thereof as follows:

4-(4-(methylsulfonyl)phenyl]-3-phenyl-2(5H)-furanone (rofecoxib),

4-[5-(4-methylphenyl)-3-(trifluoromethyl)-1H-pyrazol-1-yl]-benzenesulfonamide (celecoxib),

4-[5-methyl-3-phenyl-3-phenylisoxazol-4-yl]benzensulfonamide (valdecoxib),

4-[5-(3-fluoro-4mthoxyphenyl)-3-difluoromethyl)-1H-pyrazol-1-yl]bensenesulfonamide (deracoxib),

4-(4-cyclohexyl-2-methyloxazol-5-yl)-2-fluorobenzenesulfonamide (JTE-522), and

2-(6-methylpyrid-3-yl)-3-(4-methylsulfinylphenyl)-5-chloropyridine (MK-663).

As used herein any Cox-2 selective inhibiting agent which comprises a 2H-1-benzopyran structure is called a “chromene Cox-2 selective inhibiting agent.” A class of chromene selective Cox-2 inhibiting agents useful in the methods, combinations and compositions of the present invention include compounds of Formula 2.

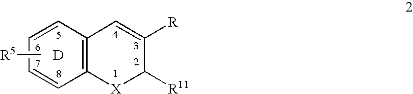

wherein

X is O, S or NR^{a};

R^{a} is alkyl;

R is carboxyl, alkyl, aralkyl, aminocarbonyl, alkylsulfonylaminocarbonyl or alkoxycarbonyl;

R¹¹ is haloalkyl, alkyl, aralkyl, cycloalkyl or aryl, wherein aryl is optionally substituted with one or more radicals selected from alkylthio, nitro and alkylsulfonyl; and

R⁵ is one or more radicals independently selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or R⁵ together with ring Dforms a naphthyl radical;

or an isomer, tautomer, pharmaceutically-acceptable salt or prodrug thereof.

Within Formula 2 there is a subclass of compounds of particular interest wherein

X is O or S;

R is carboxyl, lower alkyl, lower aralkyl or lower alkoxycarbonyl;

R¹¹ is lower haloalkyl, lower cycloalkyl or phenyl; and

R⁵ is one or more radicals independently selected from hydrido, halo, lower alkyl, lower alkoxy, lower haloalkyl, lower haloalkoxy, lower alkylamino, nitro, amino, aminosulfonyl, lower alkylaminosulfonyl, 5- or 6-membered heteroarylalkylaminosulfonyl, lower aralkylaminosulfonyl, 5- or 6-membered nitrogen containing heterocyclosulfonyl, lower alkylsulfonyl, optionally substituted phenyl, lower aralkylcarbonyl, and lower alkylcarbonyl;

or an isomer, tautomer, pharmaceutically-acceptable salt or prodrug thereof.

Preferably R is carboxyl; R¹¹ is lower haloalkyl; and R⁵ is one or more radicals independently selected from hydrido, halo, lower alkyl, lower haloalkyl, lower haloalkoxy, lower alkylamino, amino, aminosulfonyl, lower alkylaminosulfonyl, 5- or 6-membered heteroarylalkylaminosulfonyl, lower aralkylaminosulfonyl, lower alkylsulfonyl, 6-membered nitrogen containing heterocyclosulfonyl, optionally substituted phenyl, lower aralkylcarbonyl, and lower alkylcarbonyl; or an isomer, tautomer, pharmaceutically-acceptable salt or prodrug thereof.

Still other preferred compounds within Formula 2 of interest include compounds wherein R¹¹ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and R⁵ is one or more radicals independently selected from hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or an isomer, tautomer, pharmaceutically-acceptable salt or prodrug thereof.

Another preferred class of compounds within Formula 2 are compounds wherein R is carboxyl; R¹¹ is trifluoromethyl or pentafluorethyl; and R⁵ is one or more radicals independently selected from hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or an isomer, tautomer, pharmaceutically-acceptable salt or prodrug thereof.

A family of specific compounds within Formula 2 of particular interest include the following compounds and their isomers and pharmaceutically-acceptable salts:

6-chloro-2-trifluoromethyl-2H-1-benzopyran-3-carboxylic acid,

6-chloro-7-methyl-2-trifluoromethyl-2H-1-benzopyran-3-carboxylic acid,

8-(1-methylethyl)-2-trifluoromethyl-2H-1-benzopyran-3-carboxylic acid,

6-chloro-7-(1,1-dimethylethyl)-2-trifluoromethyl-2H-1-benzopyran-3-carboxylic acid,

6-chloro-8-(1-methylethyl)-2-trifluoromethyl-2H-1-benzopyran-3-carboxylic acid,

2-trifluoromethyl-3H-naphthopyran-3-carboxylic acid,

7-(1,1-dimethylethyl)-2-trifluoromethyl-2H-1-benzopyran-3-carboxylic acid,

6-bromo-2-trifluoromethyl-2H-1-benzopyran-3-carboxylic acid,

8-chloro-2-trifluoromethyl-2H-1-benzopyran-3-carboxylic acid,

6-trifluoromethoxy-2-trifluoromethyl-2H-1-benzopyran-3-carboxylic acid,

5,7-dichloro-2-trifluoromethyl-2H-1-benzopyran-3-carboxylic acid,

8-phenyl-2-trifluoromethyl-2H-1-benzopyran-3-carboxylic acid,

7,8-dimethyl-2-trifluoromethyl-2H-1-benzopyran-3-carboxylic acid,

6,8-bis(dimethylethyl)-2-trifluoromethyl-2H-1-benzopyran-3-carboxylic acid,

7-(1-methylethyl)-2-trifluoromethyl-2H-1-benzopyran-3-carboxylic acid,

7-phenyl-2-trifluoromethyl-2H-1-benzopyran-3-carboxylic acid,

6-chloro-7-ethyl-2-trifluoromethyl-2H-1-benzopyran-3-carboxylic acid,

6-chloro-8-ethyl-2-trifluoromethyl-2H-1-benzopyran-3-carboxylic acid,

6-chloro-7-phenyl-2-trifluoromethyl-2H-1-benzopyran-3-carboxylic acid,

6,7-dichloro-2-trifluoromethyl-2H-1-benzopyran-3-carboxylic acid,

6,8-dichloro-2-trifluoromethyl-2H-1-benzopyran-3-carboxylic acid,

2-trifluoromethyl-3H-naptho[2,1-b]pyran-3-carboxylic acid,

6-chloro-8-methyl-2-trifluoromethyl-2H-1-benzopyran-3-carboxylic acid,

8-chloro-6-methyl-2-trifluoromethyl-2H-1-benzopyran-3-carboxylic acid,

8-chloro-6-methoxy-2-trifluoromethyl-2H-1-benzopyran-3-carboxylic acid,

6-bromo-8-chloro-2-trifluoromethyl-2H-1-benzopyran-3-carboxylic acid,

8-bromo-6-fluoro-2-trifluoromethyl-2H-1-benzopyran-3-carboxylic acid,

8-bromo-6-methyl-2-trifluoromethyl-2H-1-benzopyran-3-carboxylic acid,

8-bromo-5-fluoro-2-trifluoromethyl-2H-1-benzopyran-3-carboxylic acid,

6-chloro-8-fluoro-2-trifluoromethyl-2H-1-benzopyran-3-carboxylic acid,

6-bromo-8-methoxy-2-trifluoromethyl-2H-1-benzopyran-3-carboxylic acid,

6-[[(phenylmethyl)amino]sulfonyl]-2-trifluoromethyl-2H-1-benzopyran-3-carboxylic acid,

6-[(dimethylamino)sulfonyl]-2-trifluoromethyl-2H-1-benzopyran-3-carboxylic acid,

6-[(methylamino)sulfonyl]-2-trifluoromethyl-2H-1-benzopyran-3-carboxylic acid,

6-[(4-morpholino)sulfonyl]-2-trifluoromethyl-2H-1-benzopyran-3-carboxylic acid,

6-[(1,1-dimethylethyl)aminosulfonyl]-2-trifluoromethyl-2H-1-benzopyran-3-carboxylic acid,

6-[(2-methylpropyl)aminosulfonyl]-2-trifluoromethyl-2H-1-benzopyran-3-carboxylic acid,

6-methylsulfonyl-2-trifluoromethyl-2H-1-benzopyran-3-carboxylic acid,

8-chloro-6-[[(phenylmethyl)amino]sulfonyl]-2-trifluoromethyl-2H-1-benzopyran-3-carboxylic acid,

6-phenylacetyl-2-trifluoromethyl-2H-1-benzopyran-3-carboxylic acid,

6,8-dibromo-2-trifluoromethyl-2H-1-benzopyran-3-carboxylic acid,

8-chloro-5,6-dimethyl-2-trifluoromethyl-2H-1-benzopyran-3-carboxylic acid,

6,8-dichloro-(S)-2-trifluoromethyl-2H-1-benzopyran-3-carboxylic acid,

6-benzylsulfonyl-2-trifluoromethyl-2H-1-benzopyran-3-carboxylic acid,

6-[[N-(2-furylmethyl)amino]sulfonyl]-2-trifluoromethyl-2H-1-benzopyran-3-carboxylic acid,

6-[[N-(2-phenylethyl)amino]sulfonyl]-2-trifluoromethyl-2H-1-benzopyran-3-carboxylic acid,

6-iodo-2-trifluoromethyl-2H-1-benzopyran-3-carboxylic acid,

7-(1,1-dimethylethyl)-2-pentafluoroethyl-2H-1-benzopyran-3-carboxylic acid, and

6-chloro-2-trifluoromethyl-2H-1-benzothiopyran-3-carboxylic acid.

Another class of chromene selective Cox-2 inhibiting agents useful in the methods, combinations and compositions of the present invention include compounds of Formula 3:

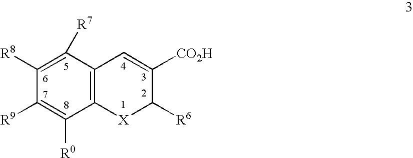

wherein

X is O, S or NR^{a};

R^{a} is alkyl;

R⁶ is lower haloalkyl;

R⁷ is hydrido or halo;

R⁸ is hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen containing heterocyclosulfonyl;

R⁹ is hydrido, lower alkyl, halo, lower alkoxy, or aryl; and

R¹⁰ is hydrido, halo, lower alkyl, lower alkoxy, or aryl;

or an isomer or pharmaceutically-acceptable salt or prodrug thereof.

Within Formula 3 there is a subclass of compounds of particular interest

wherein

R⁶ is trifluoromethyl or pentafluoroethyl;

R⁷ is hydrido, chloro, or fluoro;

R⁸ is hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R⁹ is hydrido, methyl, ethyl, isopropyl, tert-butyl, chloro, methoxy, diethylamino, or phenyl; and

R¹⁰ is hydrido, chloro, bromo, fluoro, methyl, ethyl, tert-butyl, methoxy, or phenyl;

or an isomer, tautomer, pharmaceutically-acceptable salt or prodrug thereof.

Specific compounds of interest within Formula 3 include each of the compounds and pharmaceutically-acceptable salts thereof as follows:

6-Chloro-2-trifluoromethyl-2H-1-benzopyran-3-carboxylic acid,

(S)-6-Chloro-2-trifluoromethyl-2H-1-benzopyran-3-carboxylic acid,

6-Chloro-7-(1,1-dimethylethyl)-2-trifluoromethyl-2H-1-benzopyran-3-carboxylic acid,

(S)-6-Chloro-7-(1,1-dimethylethyl)-2-(trifluoromethyl)-2H-1-benzopyran-3-carboxylic acid,

6-Trifluoromethoxy-2-trifluoromethyl-2H-1-benzopyran-3-carboxylic acid,

(S)-6-Trifluoromethoxy-2-trifluoromethyl-2H-1-benzopyran-3-carboxylic acid,

6-Formyl-2-(trifluoromethyl)-2H-1-benzopyran-3-carboxylic acid,

6-(Difluoromethyl)-2-(trifluoromethyl)-2H-1-benzopyran-3-carboxylic acid,

6,8-Dichloro-7-methyl-2-(trifluoromethyl)-2H-1-benzopyran-3-carboxylic acid,

6,8-Dichloro-2-trifluoromethyl-2H-1-benzopyran-3-carboxylic acid,

(S)-6,8-Dichloro-2-(trifluoromethyl)-2H-1-benzopyran-3-carboxylic acid,

6-Chloro-1,2-dihydro-2-(trifluoromethyl)-3-quinolinecarboxylic acid,

(S)-6-chloro-1,2-dihydro-2-(trifluoromethyl)-3-quinolinecarboxylic acid,

6,8-Dichloro-1,2-dihydro-2-(trifluoromethyl)-3-quinolinecarboxylic acid,

7-(1,1-Dimethylethyl)-2-trifluoromethyl-2H-1-benzopyran-3-carboxylic acid,

6,7-Dichloro-2-trifluoromethyl-2H-1-benzopyran-3-carboxylic acid,

5,6-Dichloro-2-(trifluoromethyl)-2H-1-benzopyran-3-carboxylic acid,

2,6-Bis(trifluoromethyl)-2H-1-benzopyran-3-carboxylic acid,

5,6,7-Trichloro-2-(trifluoromethyl)-2H-1-benzopyran-3-carboxylic acid,

6,7,8-Trichloro-2-(trifluoromethyl)-2H-1-benzopyran-3-carboxylic acid,

6-Iodo-1,2-dihydro-2-(trifluoromethyl)-3-quinolinecarboxylic acid,

6-Bromo-1,2-dihydro-2-(trifluoromethyl)-3-quinolinecarboxylic acid,

6-Chloro-7-methyl-2-(trifluoromethyl)-2H-1-benzothiopyran-3-carboxylic acid, and

6,8-Dichloro-2-trifluoromethyl-2H-1-benzothiopyran-3-carboxylic acid.

Specific compounds of particular interest within Formula 3 include each of the compounds and pharmaceutically-acceptable salts thereof as follows:

6-Chloro-2-trifluoromethyl-2H-1-benzopyran-3-carboxylic acid,

(S)-6-Chloro-2-trifluoromethyl-2H-1-benzopyran-3-carboxylic acid,

6-Chloro-7-(1,1-dimethylethyl)-2-trifluoromethyl-2H-1-benzopyran-3-carboxylic acid,

(S)-6-Chloro-7-(1,1-dimethylethyl)-2-(trifluoromethyl)-2H-1-benzopyran-3-carboxylic acid,

6-Trifluoromethoxy-2-trifluoromethyl-2H-1-benzopyran-3-carboxylic acid,

(S)-6-Trifluoromethoxy-2-trifluoromethyl-2H-1-benzopyran-3-carboxylic acid,

6-Formyl-2-(trifluoromethyl)-2H-1-benzopyran-3-carboxylic acid,

6-(Difluoromethyl)-2-(trifluoromethyl)-2H-1-benzopyran-3-carboxylic acid,

6,8-Dichloro-7-methyl-2-(trifluoromethyl)-2H-1-benzopyran-3-carboxylic acid,

6,8-Dichloro-2-trifluoromethyl-2H-1-benzopyran-3-carboxylic acid,

(S)-6,8-Dichloro-2-(trifluoromethyl)-2H-1-benzopyran-3-carboxylic acid,

6-Chloro-1,2-dihydro-2-(trifluoromethyl)-3-quinolinecarboxylic acid,

(S)-6-chloro-1,2-dihydro-2-(trifluoromethyl)-3-quinolinecarboxylic acid, and

6,8-Dichloro-1,2-dihydro-2-(trifluoromethyl)-3-quinolinecarboxylic acid.

Other selective cyclooxygenease-2 inhibiting agents useful in the methods, combinations and compositions of the present invention include compounds and pharmaceutically-acceptable salts thereof as follows:

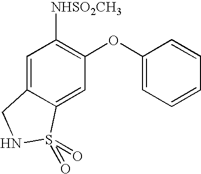

N-(2,3-dihydro-1,1-dioxido-6-phenoxy-1,2-benzisothiazol-5-yl)methanesulfonamide;

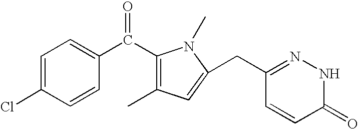

6-[[5-(4-chlorobenzoyl)-1,4-dimethyl-1H-pyrrol-2-yl]methyl]-3(2H)-pyridazinone;

ABT-963, 2-(3,4-difluorophenyl)-4-(3-hydroxy-3-methylbutoxy)-5-[4-(methylsulfonyl)phenyl]-3(2H)-pyridazinone;

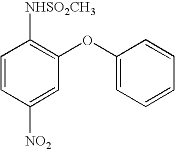

N-(4-nitro-2-phenoxyphenyl)methanesulfonamide;

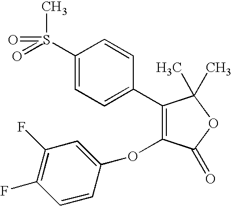

3-(3,4-difluorophenoxy)-5,5-dimethyl-4-[4-(methylsulfonyl)phenyl]-2(5H)-furanone;

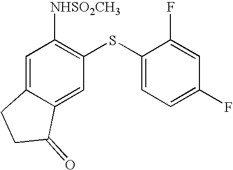

N-[6-[(2,4-difluorophenyl)thio]-2,3-dihydro-1-oxo-1H-inden-5-yl]methanesulfonamide;

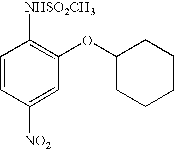

N-[2-(cyclohexyloxy)-4-nitrophenyl]methanesulfonamide;

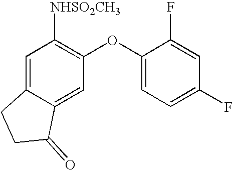

N-[6-(2,4-difluorophenoxy)-2,3-dihydro-1-oxo-1H-inden-5-yl]methanesulfonamide;

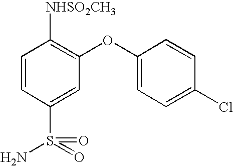

3-(4-chlorophenoxy)-4-[(methylsulfonyl)amino]benzenesulfonamide;

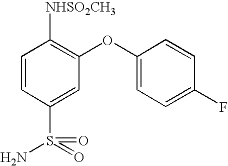

3-(4-fluorophenoxy)-4-[(methylsulfonyl)amino]benzenesulfonamide;

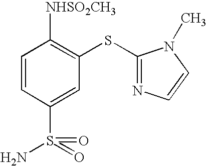

3-[(1-methyl-1H-imidazol-2-yl)thio]-4 [(methylsulfonyl) amino]benzenesulfonamide;

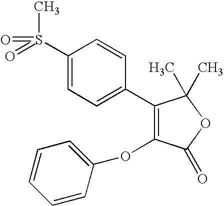

5,5-dimethyl-4-[4-(methylsulfonyl)phenyl]-3-phenoxy-2(5H)-furanone;

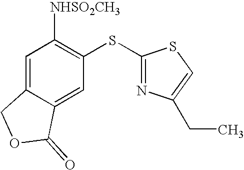

N-[6-[(4-ethyl-2-thiazolyl)thio]-1,3-dihydro-1-oxo-5-isobenzofuranyl]methanesulfonamide;

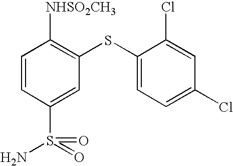

3-[(2,4-dichlorophenyl)thio]-4-[(methylsulfonyl)amino]benzenesulfonamide;

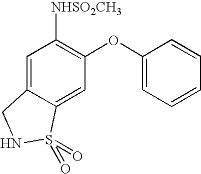

N-(2,3-dihydro-1,1-dioxido-6-phenoxy-1,2-benzisothiazol-5-yl)methanesulfonamide; and

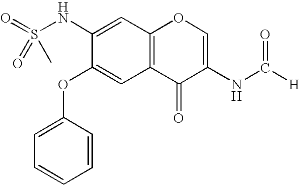

N-[3-(formylamino)-4-oxo-6-phenoxy-4H-1-benzopyran-7-yl]methanesulfonamide.

Nonlimiting examples of Cox-2 selective inhibiting agents that may be used in the methods, combinations and compositions of the present invention are identified in Table 1 below.

TABLE 1
Cox-2 Inhibitors

| | Trade | | |
|---|---|---|---|
| Compound | Name | Reference | Dosage |
| 6-chloro-4-hydroxy-2- | Iornoxicam; | CAS No. | |
| methyl-N-2-pyridinyl-2H- | Safem ® | 70374-39-9 | |
| thieno[2,3-e]-1,2-thiazine- | | | |
| 3-carboxamide, 1,1- | | | |
| dioxide | | | |
| 1,5-Diphenyl-3- | | WO | |
| substituted pyrazoles | | 97/13755 | |
| | radicicol | WO | |
| | | 96/25928; | |
| | | Kwon et al | |
| | | (Cancer | |
| | | Res(1992) 52 | |
| | | 6296) | |
| | GB- | | |
| | 02283745 | | |
| | TP-72 | Cancer Res. | |
| | | 1998 58 4 | |
| | | 717-723 | |
| 1-(4-chlorobenzoyl)-3-[4- | A- | | |
| (4-fluorophenyl )thiazol-2- | 183827.0 | | |
| ylmethyl]-5-methoxy-2- | | | |
| methy lindole | | | |
| | GR- | CAS Registry | |
| | 253035 | No. 215522- | |
| | | 99-9 | |
| 4-(4-cyclohexyl-2- | JTE-522 | CAS Registry | |
| methyloxazol-5-yl)-2- | | Number: | |
| fluorobenzenesulfonamide; | | 180200-68-4; | |
| Benzenesulfonamide, | | JP 09052882 | |
| 4-(4-cyclohexyl-2-methyl- | | | |
| 5-oxazolyl)-2-fluoro- | | | |
| 5-chloro-3-(4- | | | |
| (methylsulfonyl)phenyl)- | | | |
| 2-(methyl-5- | | | |
| pyridinyl)pyridine | | | |
| 2-(3,5-difluorophenyl)-3- | | | |
| 4- | | | |
| (methylsulfonyl)phenyl)- | | | |
| 2-cyclopenten-1-one | | | |
| 5-[4-(methylsulfonyl)- | L-768277 | CAS Registry | |
| phenyl]-6-phenyl- | | No. 180696- | |
| thiazolo[3,2- | | 49-5 | |
| b][1,2,4]triazole | | | |
| | L-783003 | CAS Registry | |
| | | No. 215435- | |
| | | 69-1 | |
| 4-(4-(methyl- | MK-966; | U.S. Pat. No. | 12.5-100 |
| sulfonyl)phenyl]-3-phenyl- | Vioxx ®; | 5968974 | mg po |
| 2(5H)-furanone; | rofecoxib | | |
| indomethacin-derived | | WO | 200 |
| indolalkanoic acid | | 96/37467-9 | g/kg/day |
| 1-Methylsulfonyl-4-[1,1- | | WO | |
| dimethyl-4-(4- | | 95/30656; | |
| fluorophenyl)cyclopenta- | | WO | |
| 2,4-dien-3-yl]benzene | | 95/30652; | |
| | | WO | |
| | | 96/38418; | |
| | | WO | |
| | | 96/38442 | |
| 4,4-dimethyl-2-phenyl-3- | | | |
| [4- | | | |
| (methylsulfonyl)phenyl] | | | |
| cyclobutenone | | | |
| 2-(4-methoxyphenyl)-4- | | EP 799823 | |
| methyl-1-(4- | | | |
| sulfamoylphenyl)pyrrole | | | |
| N-[5-(4- | RWJ- | | |
| fluoro)phenoxy]thiophene- | 63556 | | |
| 2-methanesulfonamide | | | |
| 5(E)-(3,5-di-tert-butyl-4- | S-2474 | EP 595546 | |
| hydroxy)benzylidene-2- | | | |
| ethyl-1,2-isothiazolidine- | | | |
| 1,1-dioxide | | | |
| 3-formylamino-7- | T-614 | DE 3834204 | |
| methylsulfonylamino-6- | | | |
| phenoxy-4H-1- | | | |
| benzopyran-4-one | | | |
| Benzenesulfonamide, 4- | celecoxib; | CAS Registry | |
| (5-(4-methylphenyl)-3- | Celebrex ® | Number: | |
| (trifluoromethyl)-1H- | | 169590-42-5; | |
| pyrazol-1-yl)- | | U.S. Pat. No. | |
| | | 5466823 | |
| Benzenesulfonamide, 4- | valdecoxib | CAS Registry | |
| (5-methyl-3-phenyl-4- | | Number: | |
| isoxazolyl)- | | 181695-72-7; | |
| | | 5,633,272 | |
| Propanamide, N-[[4-(5- | parecoxib | CAS Registry | |
| methyl-3-phenyl-4- | (prodrug) | Number: | |
| isoxazolyl)phenyl]sulfonyl]- | | 198470-84-7; | |
| | | U.S. Pat. No. | |
| | | 5932598 | |
| 4-[5-(3-fluoro-4- | deracoxib | CAS Registry | |
| methoxyphenyl)-3- | | Number: | |
| difluoromethyl)-1H- | | 169590-41-4; | |
| pyrazol-1- | | U.S. Pat. No. | |
| yl]benzenesulfonamide | | 5521207 | |
| | meloxicam | U.S. Pat. No. | 15-30 |
| | | 4233299 | mg/day |
| | nimesulide | U.S. Pat. No. | |
| | | 3840597 | |
| 1,5-Diphenyl-3- | | WO | |
| substituted pyrazoles | | 97/13755 | |
| | radicicol | WO 96/25928. | |
| | | Kwon et al | |
| | | (Cancer | |
| | | Res(1992) 52 | |
| | | 6296) | |
| | TP-72 | Cancer Res. | |
| | | 1998 58 4 | |
| | | 717-723 | |
| 1-(4-chlorobenzoyl)-3-[4- | A- | | |
| (4-fluoro-phenyl )thiazol- | 183827.0 | | |
| 2-ylmethyl]-5-methoxy-2- | | | |
| methy lindole | | | |
| | GR- | | |
| | 253035 | | |
| 5-chloro-3-(4- | | | |
| (methylsulfonyl)phenyl)- | | | |
| 2-(methyl-5-pyridinyl)- | | | |
| pyridine | | | |
| 2-(3,5-difluoro-phenyl)-3- | | | |
| 4-(methylsulfonyl)- | | | |
| phenyl)-2-cyclopenten-1- | | | |
| one | | | |
| CS 502 | Sankyo | | |
| 2-(6-methylpyrid-3-yl)-3- | etoricoxib; | WO | |
| (4-methylsulfinylphenyl)- | MK-663; L- | 98/03484; | |
| 5-chloropyridine | 791456 | Bioorg. Med. | |
| | | Chem. Lett. | |
| | | 1998, 8, | |
| | | 2777-2782 | |

The following individual references listed in Table No. 2 below, each hereby incorporated by reference, describe various Cox-2 selective inhibiting agents suitable for use in the methods, combinations and compositions of the present invention described herein, and processes for their manufacture.

TABLE No. 2
Cox-2 Inhibitor References

| WO 99/30721 | WO 99/30729 | U.S. Pat. No. | WO 98/15528 |
|---|---|---|---|
| | | 5760068 | |
| WO 99/25695 | WO 99/24404 | WO 99/23087 | FR 27/71005 |
| EP 921119 | FR 27/70131 | WO 99/18960 | WO 99/15505 |
| WO 99/15503 | WO 99/14205 | WO 99/14195 | WO 99/14194 |
| WO 99/13799 | GB 23/30833 | U.S. Pat. No. | WO 99/12930 |
| | | 5859036 | |
| WO 99/11605 | WO 99/10332 | WO 99/10331 | WO 99/09988 |
| U.S. Pat. No. | WO 99/05104 | U.S. Pat. No. | WO 98/47890 |
| 5869524 | | 5859257 | |
| WO 98/47871 | U.S. Pat. No. | U.S. Pat. No. | WO 98/45294 |
| | 5830911 | 5824699 | |
| WO 98/43966 | WO 98/41511 | WO 98/41864 | WO 98/41516 |
| WO 98/37235 | EP 86/3134 | JP 10/175861 | U.S. Pat. No. |
| | | | 5776967 |
| WO 98/29382 | WO 98/25896 | ZA 97/04806 | EP 84/6,689 |
| WO 98/21195 | GB 23/19772 | WO 98/11080 | WO 98/06715 |
| WO 98/06708 | WO 98/07425 | WO 98/04527 | WO 98/03484 |
| FR 27/51966 | WO 97/38986 | WO 97/46524 | WO 97/44027 |
| WO 97/34882 | U.S. Pat. No. | WO 97/37984 | U.S. Pat. No. |
| | 5681842 | | 5686460 |
| WO 97/36863 | WO 97/40012 | WO 97/36497 | WO 97/29776 |
| WO 97/29775 | WO 97/29774 | WO 97/28121 | WO 97/28120 |
| WO 97/27181 | WO 95/11883 | WO 97/14691 | WO 97/13755 |
| WO 97/13755 | CA 21/80624 | WO 97/11701 | WO 96/41645 |
| WO 96/41626 | WO 96/41625 | WO 96/38418 | WO 96/37467 |
| WO 96/37469 | WO 96/36623 | WO 96/36617 | WO 96/31509 |
| WO 96/25405 | WO 96/24584 | WO 96/23786 | WO 96/19469 |
| WO 96/16934 | WO 96/13483 | WO 96/03385 | U.S. Pat. No. |
| | | | 5510368 |
| WO 96/09304 | WO 96/06840 | WO 96/06840 | WO 96/03387 |
| WO 95/21817 | GB 22/83745 | WO 94/27980 | WO 94/26731 |
| WO 94/20480 | WO 94/13635 | FR 27/70,131 | U.S. Pat. No. |
| | | | 5859036 |
| WO 99/01131 | WO 99/01455 | WO 99/01452 | WO 99/01130 |
| WO 98/57966 | WO 98/53814 | WO 98/53818 | WO 98/53817 |
| WO 98/47890 | U.S. Pat. No. | U.S. Pat. No. | WO 98/22101 |
| | 5830911 | 5776967 | |
| DE 19/753463 | WO 98/21195 | WO 98/16227 | U.S. Pat. No. |
| | | | 5733909 |
| WO 98/05639 | WO 97/44028 | WO 97/44027 | WO 97/40012 |
| WO 97/38986 | U.S. Pat. No. | WO 97/34882 | WO 97/16435 |
| | 5677318 | | |
| WO 97/03678 | WO 97/03667 | WO 96/36623 | WO 96/31509 |
| WO 96/25928 | WO 96/06840 | WO 96/21667 | WO 96/19469 |
| U.S. Pat. No. | WO 96/09304 | GB 22/83745 | WO 96/03392 |
| 5510368 | | | |
| WO 94/25431 | WO 94/20480 | WO 94/13635 | JP 09052882 |
| GB 22/94879 | WO 95/15316 | WO 95/15315 | WO 96/03388 |
| WO 96/24585 | U.S. Pat. No. | WO 95/00501 | U.S. Pat. No. |
| | 5344991 | | 5968974 |
| U.S. Pat. No. | U.S. Pat. No. | U.S. Pat. No. | |
| 5945539 | 5994381 | 5521207 | |

The rofecoxib used in the therapeutic methods, combinations and compositions of the present invention can be prepared in the manner set forth in U.S. Pat. No. 5,968,974.

The celecoxib used in the therapeutic methods, combinations and compositions of the present invention can be prepared in the manner set forth in U.S. Pat. No. 5,466,823.

The valdecoxib used in the therapeutic methods, combinations and compositions of the present invention can be prepared in the manner set forth in U.S. Pat. No. 5,633,272.

The parecoxib used in the therapeutic methods, combinations and compositions of the present invention can be prepared in the manner set forth in U.S. Pat. No. 5,932,598.

The deracoxib used in the therapeutic methods, combinations and compositions of the present invention can be prepared in the manner set forth in U.S. Pat. No. 5,521,207.

The Japan Tobacco JTE-522 used in the therapeutic methods, combinations and compositions of the present invention can be prepared in the manner set forth in JP 90/52,882.

The etoricoxib used in the therapeutic methods, combinations and compositions of the present invention can be prepared in the manner set forth in WO document WO 98/03484.

A DNA topoisomerase I inhibitor, or a DNA topoisomerase I inhibiting agent, encompass a wide range of structures that are useful in the methods, combinations and compositions of the present invention. A compound that inhibits DNA topoisomerase I is used in combination with a Cox-2 selective inhibiting agent to practice the present invention. Compounds which have inhibitory activity for DNA topoisomerase I can be readily identified by using assays well-known in the art.

Topoisomerase I is a monomeric nuclear enzyme of 100 kDa involved in DNA replication, RNA transcription, mitosis, chromosome condensation, and probably DNA repair. Topoisomerase I forms a covalent complex with DNA which allows the formation of the single-strand breaks necessary for DNA replication. Topoisomerase I also religates those DNA strands after DNA replication. While not wishing to be bound by theory, it is believed that DNA topoisomerase I inhibiting agents bind to this DNA topoisomerase I complex in a reversible manner, resulting in the inhibition of topoisomerase I action. DNA topoisomerase I inhibiting agents have been shown to not only bind to the topoisomerase I enzyme but also to the DNA.

DNA topoisomerase I inhibiting agents of particular interest that can be used with the methods, combinations and compositions of the present invention are provided in Table No. 3 below. The therapeutic compounds of Table No. 3 can be used in the methods, combinations and compositions of the present invention in a variety of forms, including acid form, salt form, racemates, enantiomers, zwitterions, and tautomers. The individual references in Table No. 3 are each herein individually incorporated by reference.

TABLE 3
DNA Topoisomerase I Inhibitors

| | | | | | Oncology |
|---|---|---|---|---|---|
| Compound Name | Trade Name | Reference | Dosage | Toxicity | Indication |
| | Camptothecin | WO 9637496 | | Myelo- | Colon, stomach, and |
| | | J. Am. Chem. Soc. | | suppression, | non-small cell lung |
| | | 1966; 88: 3888-90. | | nausea, vomiting, | cancer. Melanoma. |
| | | | | and diarrhea, and | |
| | | | | hemorrhagic | |
| | | | | cystitis. | |
| 9-amino-20(S)- | | Cancer Res. 1989; | | | Colon, non-small |
| camptothecin | | 49: 1465-1469. | | | cell lung, and breast |
| | | Cancer Res. 1989; | | | cancer. Melanoma. |
| | | 49: 4385-4389 | | | |
| | GG211 | Proc Am Assoc. | | Hematologic | Colon, ovarian, lung |
| | | Cancer Res. 1994; | | toxicity dose | and epidermoid |
| | | 35: 47. | | limiting. | cancer. |
| | Irinotecan | Cancer Res. 1991; | 20 mg/m² for 3 | Diarrhea and | Colon, head and |
| | | 51: 4187-4191. | days weekly; | myelo- | neck, non-small cell |
| | | Cancer Res. 1987; | 100 mg/m² | suppression. | lung, cervical, |
| | | 47: 5944-5947. | weekly; 150 | | esophageal, renal |
| | | Cancer Res. 1990; | mg/ m² every 2 | | cell, breast, and |
| | | 50: 1715-1720. | weeks; 200 | | ovarian cancer. |
| | | | mg/m² every 3- | | Gastric and lung |
| | | | 4 weeks; 250 | | squamous cell |
| | | | mg/m² every 3- | | carcinomas. |
| | | | 4 weeks | | Rhabdomysarcoma. |
| | | | | | Non-Hodgkin's |
| | | | | | lymphoma. Combi- |
| | | | | | nation therapy: |
| | | | | | Recombinant |
| | | | | | granulocyte colony |
| | | | | | stimulating factor |
| | | | | | (G-CSF). 5- |
| | | | | | fluorouracil. |
| | | | | | Cisplatin Etoposide |
| (4S)-4,11-diethyl- | Irinotecan hydro- | U.S. Pat. No. | 125 mg/ m² IV | Lethality in mice: | Metastatic |
| 4-hydroxy-9-((4- | chloride, CPT-11. | 4604463. | over 90 | 111 mg/kg in mice. | carcinoma of the |
| piperi- | Camptosar ® Injection | EP 56692. | minutes/wk for | Lethality in rats: 73 | colon or rectum. |
| dinopiperidino)car | | JP 60019790. | 4 weeks | mg/kg. DLT: | Brain tumor, |
| bonyloxy)-1H- | | | followed by 2 | diarrhea and | arcinoma, Lung |
| pyrano(3′,4′:6,7)in- | | | week rest. | neutropenia. | tumor, Neoplasm, |
| dolizino(1,2- | | | Then repeated | Myelosup- | Non-Hodgkin |
| b)quinoline- | | | at 50 to 150 | pression, | lymphoma, Non- |
| 3,14(4H, 12H)dione | | | mg/m² doses. | neutropenia, | small-cell lung |
| hydrochloride. | | | | leukopenia | cancer, Ovary |
| | | | | (including lympho- | tumor, Pancreas |
| | | | | cytopenia), and | tumor, Stomach |
| | | | | anemia. | tumor, Uterine cervix |
| | | | | | tumor, Uterus tumor. |
| (S)-10- | Topotecan | | 1.5 mg/ m²/d | DLT: Bone marrow | Metastatic |
| ((dimethylamino) | hydrochloride; | | IV infusion | suppression. | carcinoma of the |
| methyl)-4-ethyl- 4,9- | Hycamtin | | over 30 | LD10: mice 75 | ovary. |
| dihydroxy-1H- | | | minutes for 5 | mg/m² single IV | Radio/chemosensiti- |
| pyrano (3′, | | | consecutive | infusion. Grade 4 | zer; Breast tumor, |
| 4′:6,7)indolizino(1, | | | days, starting | thrombo- | Carcinoma, Colon |
| 2-B)quinoline- | | | on day one of | cytopenia, anemia. | tumor, Glioma, |
| 3,14-(4H, 12H)- | | | a 21-day | | Leukemia, Lung |
| dione | | | course. | | tumor, Lymphoma, |
| monohydrochloride | | | | | Myeloproliferative |
| | | | | | disorder. |
| 1H- | Topotecan | EP 321122. | 1.5 mg/m² × 5 | Maximally | Colorectal, small |
| Pyrano[3′,4′:6,7]in- | | | d every 3 wk: | tolerated dose: 1.5 | and non-small cell |
| dolizino[1,2- | | | Prostate, | mg/m² × 5 d every | lung cancer; |
| b]quinoline- | | | colorectal, and | 3 to 4 wk. | ovarian, |
| 3,14(4, H, 12H)- | | | ovarian cancer. | Myelosuppression | esophageal, renal, |
| dione, 10- | | | 1.5 mg/m² × 5 | dose-limiting | squamous cell skin, |
| [(dimethylamino) | | | d every 4 wk: | toxicity. | prostate, and |
| methyl]- | | | Renal cell | Subsequent | epidermoid cancer. |
| 4-ethyl-4,9- | | | cancer. | administration of | Osteogenic |
| dihydroxy-, (S)- | | | | G-CSF lowers | sarcoma, rhabdo- |
| | | | | severity of neutro- | mysarcoma, acute |
| | | | | penia, allowing | myelo-blastic |
| | | | | dose escaltion. | leukemia, chronic |
| | | | | | myelocytic leukemia |
| | | | | | in blastic phase. |
| | | | | | Leiomyosarcoma. |
| | | | | | Combination |
| | | | | | therapy: Etoposide |
| | | | | | and cisplatin. |
| MAG- | PNU-166148 | Proc Am Soc. Clin | | | Solid tumors, |
| camptothecin | | Oncol 2000 19 | | | |
| (prodrug) | | May 20-23 Abs | | | |
| | | 771 | | | |
| 11H-1,4- | lurtotecan | EP 540099 | 0.3 to 0.5 | hematological | neoplasia |
| Dioxino[2,3- | | | mg/m2/day by | toxicity, | |
| g]pyrano[3′,4′:6,7] | | | continuous | myelotoxicity, | |
| indolizino[1,2- | | | infusions of 7, | gastrointestinal | |
| b[quinoline- | | | 14, and 21 | toxicity, | |
| 9,12(8H, 14H)- | | | days. | thrombocytopenia | |
| dione, 8-ethyl- | | | | and neutropenia | |
| 2,3-dihydro-8- | | | | and asthenia | |
| hydroxy-15-[(4- | | | | | |
| methyl-1- | | | | | |
| piperazinyl) | | | | | |
| methyl]-, | | | | | |
| (S)- | | | | | |
| 11H-1,4- | Lurtotecan | EP 540099 | 0.3 to 0.5 | hematological | neoplasia |
| Dioxino[2,3- | dihydrochloride | | mg/m2/day by | toxicity, | |
| g]pyrano[3′,4′:6,7] | | | continuous | myelotoxicity, | |
| indolizino[1,2- | | | infusions of 7, | gastrointestinal | |
| b]quinoline- | | | 14, and 21 | toxicity, | |
| 9,12(8H, 14H)- | | | days. | thrombocytopenia | |
| dione, 8-ethyl- | | | | and neutropenia | |
| 2,3-dihydro-8- | | | | and asthenia | |
| hydroxy-15-[(4- | | | | | |
| methyl-1- | | | | | |
| piperazinyl) | | | | | |
| methyl]-, | | | | | |
| dihydrochloride, | | | | | |
| (S)- | | | | | |
| 1H- | 9-aminocamptothecin | | Dose limiting | Maximum | Colon tumor, Solid |
| Pyrano[3′,4′:6,7] | | | toxicity | tolerated dose = | tumor, Neoplasm, |
| indolizino[1,2- | | | consisted of | 45 mug/square | Carcinoma, Lung |
| b]quinoline- | | | neutropenia. | meter/hr; | tumor, Colorectal |
| 3,14(4H, 12H)- | | | | | tumor, Pancreas |
| dione, 10-amino-4- | | | | | tumor, Stomach |
| ethyl-4-hydroxy-, | | | | | tumor, Bladder |
| (S)- | | | | | tumor, Prostate |
| | | | | | tumor, Head & neck |
| | | | | | tumor, Renal tumor, |
| | | | | | Leukemia |
| | DB-67, | WO 99/09996 | | | Neoplasia |
| | camptothecins, | | | | |
| | homosilatecans | | | | |
| 1H- | rubitecan, 9- | Eur J Haematol | Maximum | The dose limiting | Neoplasm, |
| Pyrano[3′,4′:6,7] | nitrocamptothecin | 1994 53 4 246- | tolerated dose: | toxicity was | Pancreas tumor, |
| indolizino[1,2- | | 248. | 1.5 mg/m2/day | hematological, | Ovary tumor, |
| b]quinoline- | | Proc Am Assoc. | over five | with grade 4 | Leukemia, Solid |
| 3,14(4H, 12H)- | | Cancer Res. 1994 | consecutive | anemia in 29% of | tumor, |
| dione, -4-ethyl-4- | | 35 Abs 2712. | days repeated | patients, | Myelodysplastic |
| hydroxy-10- | | Int J Cancer 1993 | every week. | neutropenia in | Disease |
| nitro-, (S)- | | 53 5 863-871. | | 25%, and | |
| | | | | thrombocytopenia | |
| | | | | in 18%. Grade 2 or | |
| | | | | higher toxic effects | |
| | | | | occurred at each | |
| | | | | dose level: nausea | |
| | | | | and vomiting | |
| | | | | (54%), diarrhea | |
| | | | | (32%), chemical | |
| | | | | cystitis (25%), | |
| | | | | neutropenic sepsis | |
| | | | | (21%), and weight | |
| | | | | loss (18%). | |
| 7-[N-(4-methyl-1- | CT-17 | Proc Am Assoc. | | | Neoplasia |
| piperazino) | | Cancer Res. 1999 | | | |
| methylamino]- | | 40 ABS 715 | | | |
| (20S)- | | | | | |
| camptothecin | | | | | |
| camptothecin | BAY-38-3441 | Clin Cancer Res. | | | Neoplasia |
| glycoconjugates | | 1999 5 11 3862s- | | | |
| | | 3863s. | | | |
| | | Proc Am Assoc. | | | |
| | | Cancer Res. 2000 | | | |
| | | 41 April 1-5 Abs | | | |
| | | 3430. | | | |
| camptothecin | BAY-38-3444 | Clin Cancer Res. | | | Neoplasia |
| glycoconjugates | | 1999 5 11 3862s- | | | |
| | | 3863s. | | | |
| 4(3H)- | NSC-665517 | Proc Am Assoc. | | | Carcinoma |
| Quinazolinone, | | Cancer Res. 1995 | | | |
| 6,8-dibromo-2- | | 36 Abs 2659. | | | |
| methyl-3-[2-(D- | | Mol Pharmacol | | | |
| xylopyranosylamino) | | 1995 48 4 658- | | | |
| phenyl]- | | 665 | | | |
| 2-Propenamide, 2- | AG 490, Tyrphostin | | | | Neoplasia |
| cyano-3-(3,4- | AG 490 | | | | |
| dihydroxyphenyl)- | | | | | |
| N-(phenylmethyl)-, | | | | | |
| (2E)- | | | | | |
| - | | | | | |
| 2-Propenamide, 2- | AG 555, Tyrphostin | Cancer Res. 1994 | | | Neoplasia |
| cyano-3-(3,4- | AG 555 | 54 19 5138-5142. | | | |
| dihydroxyphenyl)- | | Exp Opin Ther Pat | | | |
| N-(3- | | 1998 8 12 1599- | | | |
| hydroxyphenyl | | 1625 | | | |
| propyl)-, (E)- | | | | | |
| | NSC-314622 | Proc Am Assoc. | | | Neoplasia |
| | | Cancer Res. 1996 | | | |
| | | 431. | | | |
| | | Proc Am Assoc. | | | |
| | | Cancer Res. 2000 | | | |
| | | 41 April 1-5 Abs | | | |
| | | 5186. | | | |
| | CZ-112; CZ-48 | U.S. Pat. No. | | | malignant tumors, |
| | | 5731316 | | | neoplasia |
| | HAR-7 | Nci Eortc Symp | | | Solid tumors |
| | | New Drugs | | | |
| | | Cancer Ther 1996 | | | |
| | | 9th Amsterdam | | | |
| | | Abs 444. | | | |
| | NX-211, lurtotecan | Proc Am Assoc. | | | Neoplasia |
| | liposomal | Cancer Res. 1999 | | | |
| | | 40 Abs 751. Proc | | | |
| | | Am Soc. Clin | | | |
| | | Oncol 1999 18 15- | | | |
| | | 18 May 680. | | | |
| 5H-indolo[2,3- | J 107088; ED-749 | Proc Am Assoc. | maximum | | Neoplasia |
| a]pyrrolo[3,4- | | Cancer Res. 1998 | tolerated dose: | | |
| c]carbazole- | | 39 New Orleans | 7.5 mg/m2 | | |
| 5,7(6H)-dione, 12- | | Abs 2864. | | | |
| .beta.-D- | | Ann Oncol 1998 9 | | | |
| glucopyranosyl- | | 2 043. | | | |
| 12,13-di- | | Cancer Res. 1999 | | | |
| hydro-2,10- | | 59 17 4271-4275. | | | |
| dihydroxy-6-[[2- | | Bioorg Med Chem. | | | |
| hydroxy-1- | | Lett 1999 9 23 | | | |
| (hydroxymethyl) | | 3307-3312. | | | |
| ethyl]amino]- | | | | | |
| 4- | XR-5000, DACA | U.S. Pat. No. | | infusion-related | Brain tumor, Breast |
| Acridinecarboxa- | | 05696131. | | arm pain | tumor, Carcinoma, |
| mide, N-[2- | | Journal Of | | | Colon tumor, Lung |
| (dimethylamino) | | Medicinal | | | tumor, Melanoma, |
| ethyl]-, | | Chemistry 1987 30 | | | Ovary tumor, |
| dihydrochloride | | 664-669 | | | Sarcoma, Skin |
| | | | | | tumor |
| 4- | NSC 601316 | U.S. Pat. No. | | | Brain tumor, Breast |
| Acridinecarboxa- | | 05696131. | | | tumor, Carcinoma, |
| mide, N-[2- | | Journal Of | | | Colon tumor, Lung |
| (dimethylamino) | | Medicinal | | | tumor, Melanoma, |
| ethyl]- | | Chemistry 1987 30 | | | Ovary tumor, |
| | | 664-669 | | | Sarcoma, Skin |
| | | | | | tumor |
| 9-chloro-10- | SKF-108025 | Acs 1994 207th | | | Carcinoma |
| hydroxy | | San Diego MEDI | | | |
| camptothecin | | 74 | | | |
| | CZ-105, CZ-107 | Proceedings Of | | | Neoplasia |
| | | The American | | | |
| | | Association Of | | | |
| | | Cancer Research | | | |
| | | 1997 38 88 17 | | | |
| | JSKIV-47 | U.S. Pat. No. | | | Neoplasia |
| | | 05767142. | | | |
| | | WO 96/36612 | | | |
| | SKF-107874 | Acs Meeting 1994 | | | Carcinoma |
| | | 207th San Diego | | | |
| | | MEDI 74 | | | |
| | Intoplicine | EP 402232 | | | Solid tumor |
| | CKD-602 | WO 96/21666. | | | Neoplasia |
| | Exetacan mesylate; | EP 737686 | | | Leukemia, Myeloid |
| | exatecan | | | | leukemia, |
| | | | | | Neoplasm, Non- |
| | | | | | small-cell lung |
| | | | | | cancer, Pancreas |
| | | | | | tumor |
| | IST-622 | EP 159708 | | | Neoplasia |
| | NB-506 | WO 93/11145 | | | Neoplasia |
| | Pyrazoloacridine, | EP 138302 | | | Neoplasia |
| | Parke-Davis | | | | |
| | XR-5000 | U.S. Pat. No. | | | Brain tumor, Breast |
| | | 5696131 | | | tumor, Carcinoma, |
| | | | | | Colon tumor, Lung |
| | | | | | tumor, Melanoma, |
| | | | | | Ovary tumor, |
| | | | | | Sarcoma, Skin |
| | | | | | tumor |
| | DB-67 | WO 99/09996 | | | Neoplasia |
| | DRF-1042 | WO 97/46563 | | | Neoplasia |
| | F-11782 | WO 96/12727 | | | Neoplasia |
| | XR-5944 | WO 98/17650 | | | Neoplasia |
| | BN-80915 | WO 99/11646. | | | Neoplasia |

Other DNA topoisomerase I inhibiting agents of interest that can be used in the methods, combinations and compositions of the present invention include the compounds described in the patents provided in Table No. 4, below. The therapeutic compounds of Table No. 4 can also be used in the methods, combinations and compositions of the present invention in a variety of forms, including acid form, salt form, racemates, enantiomers, zwitterions, and tautomers. The individual references in Table No. 4 are each herein individually incorporated by reference.

TABLE No. 4
Additional DNA Topoisomerase I Inhibitors
Table 4: Additional DNA Topoisomerase I Inhibitors

| Company | Reference | Oncology Indication |
|---|---|---|
| Abbott Laboratories | WO 97/15676 | Neoplasm |
| Arch Development Corp | WO 96/01127 | Neoplasm |
| Banyu Pharmaceutical Co. | EP 388956 | Neoplasm |
| Ltd.. | | |
| Bayer AG | WO 98/14459 | Neoplasm |
| Bayer AG | WO 98/14468 | Neoplasm, Lung tumor |
| Bayer AG | WO 98/15573 | Neoplasm |
| Bayer AG | WO 98/51703 | Neoplasm |
| BioNumerik | U.S. Pat. No. | Neoplasm |
| Pharmaceuticals Inc. | 5597829 | |
| BioNumerik | WO 95/17187 | Neoplasm |
| Pharmaceuticals Inc. | | |
| BioNumerik | WO 95/29677 | Neoplasm |
| Pharmaceuticals Inc | | |
| BioNumerik | WO 98/04557 | Leukemia, Breast tumor, |
| Pharmaceuticals Inc. | | Colon tumor, Melanoma, |
| | | Lung tumor, Non-Hodgkin |
| | | lymphoma, Ovary tumor |
| BioNumerik | WO 98/35940 | Neoplasm, Leukemia |
| Pharmaceuticals Inc. | | |
| BioNumerik | WO 95/28404 | Neoplasm |
| Pharmaceuticals Inc. | | |
| Bristol-Myers Co. | BE-900735 | Carcinoma |
| Bristol-Myers Squibb Co. | WO 98/07433 | Neoplasm |
| Chong Kun Dang Corp. | WO 96/21666 | Neoplasm, Leukemia |
| Chong Kun Dang Corp. | WO 99/02530 | Neoplasm |
| Daiichi Seiyaku Co Ltd. | JP-9020778 | Carcinoma |
| Dana-Farber Cancer | WO 97/07797 | Prostate disease, Ovary |
| Institute Inc. | | tumor, Breast tumor |
| Dr Reddys Research | WO 97/46562 | Leukemia, HIV infection |
| Foundation | | |
| FermaLogic Inc. | U.S. Pat. No. | Colon tumor |
| | 5554519 | |
| George Washington | WO 99/65493 | Diarrhea, Breast tumor, |
| University | | Ovary tumor, Colon tumor, |
| | | Melanoma, Lung tumor, |
| | | Thyroid tumor, Lymphoma, |
| | | Leukemia |
| Dr Reddys Research | WO 97/46564 | Leukemia, Neoplasm |
| Foundation | | |
| Glaxo Inc. | EP 540099 | Neoplasm |
| Glaxo Inc. | GB-2280674 | Carcinoma, Neoplasm |
| Glaxo Inc. | WO 94/25466 | Neoplasm |
| | WO 96/11005 | Neoplasm |
| Istituto Nazionale | WO 97/31003 | Neoplasm |
| studio e cura dei tumori | | |
| Johns Hopkins University | WO 96/08249 | Trypanosomiasis, |
| | | Leishmania infection |
| Kaken Pharmaceutical Co. | JP-11246469 | Neoplasm |
| Ltd. | | |
| Kyorin Pharmaceutical | WO 96/41806 | Neoplasm |
| Co. Ltd. | | |
| Matrix Pharmaceutical | WO 98/36776 | Neoplasm |
| Inc. | | |
| Ohio State University | U.S. Pat. No. | Neoplasm |
| | 5552156 | |
| Pharmacia & Upjohn SpA | WO 95/22549 | Neoplasm |
| Pharmacia & Upjohn SpA | WO 95/32207 | Leukemia, Colon tumor |
| Pharmacia & Upjohn SpA | WO 97/25332 | Neoplasm |
| Pharmacia & Upjohn SpA | WO 98/35969 | Carcinoma, Leukemia |
| Pharmacia & Upjohn SpA | WO 99/17804 | Neoplasm |
| Pharmacia & Upjohn SpA | WO 95/04736 | Neoplasm, Leukemia |
| Pharmacia & Upjohn SpA | WO 99/05103 | Neoplasm |
| Pharmacia & Upjohn SpA | WO 99/17805 | Neoplasm |
| Pharmacia Inc. | WO 96/11669 | Neoplasm, Leukemia |
| Research Triangle | WO 96/02546 | Neoplasm |
| Institute | | |
| Research Triangle | WO 91/04260 | Neoplasm |
| Institute | | |
| Research Triangle | WO 91/05556 | Colorectal tumor, |
| Institute | | Leukemia, Colon tumor |
| Research Triangle | WO 96/09049 | Plasmodium infection |
| Institute | | |
| Research Triangle | WO 97/19085 | Neoplasm, Leukemia, |
| Institute | | Colon tumor |
| Rockefeller University | WO 97/44492 | Neoplasm |
| Rutgers University | U.S. Pat. No. | Neoplasm, Burkitts |
| | 5767142 | lymphoma, |
| | | Myeloproliferative |
| | | disorder, Breast tumor |
| Rutgers University | WO 98/31673 | Neoplasm, Fungal |
| | | infection |
| Rutgers University | WO 99/31067 | Malignant neoplastic |
| | | disease, Solid tumor, |
| | | Leukemia |
| Rutgers University | WO 99/41241 | Malignant neoplastic |
| | | disease, Solid tumor, |
| | | Leukemia, Lymphoma, |
| | | Fungal infection |
| Rutgers University | WO 98/12181 | Leukemia, Melanoma, |
| | | Carcinoma |
| Rutgers University | WO 99/33824 | Solid tumor, Sarcoma, |
| | | Melanoma, Lymphoma |
| Sankyo Co Ltd. | JP-7316091 | Neoplasm |
| Shionogi & Co Ltd. | JP-7138165 | Carcinoma |
| SmithKline Beecham Corp. | EP 835938 | Staphylococcus infection |
| SmithKline Beecham Corp. | U.S. Pat. No. | Solid tumor |
| | 5633016 | |
| SmithKline Beecham Corp. | U.S. Pat. No. | Ovary tumor |
| | 5674872 | |
| SmithKline Beecham Corp. | WO 92/14469 | Neoplasm, Ovary tumor |
| SmithKline Beecham Corp. | WO 95/03803 | Viral infection |
| SmithKline Beecham Corp. | WO 96/38146 | Neoplasm |
| SmithKline Beecham Corp. | WO 96/38449 | Neoplasm |
| SmithKline Beecham Corp. | WO 92/05785 | Neoplasm |
| SmithKline Beecham Corp. | WO 92/14471 | Neoplasm |
| SmithKline Beecham Corp. | WO 92/14470 | Esophageal disease, |
| | | Neoplasm |
| SmithKline Beecham plc | WO 92/07856 | Viral infection |
| Societe de Conseils de | WO 98/28305 | Colon tumor, Lung tumor, |
| Recherches et | | Breast tumor, viral |
| d'Applications | | infection, Parasitic |
| Scientifique | | infection |
| Societe de Conseils de | WO 99/33829 | Colon tumor, Lung tumor, |
| Recherches et | | Leukemia, Leishmania |
| d'Applications | | infection, Plasmodium |
| Scientifique | | infection, Trypanosomiasis |
| Stehlin Foundation For | WO 97/28165 | Neoplasm, Carcinoma, |
| Cancer Research | | Breast tumor |
| Takeda Chemical | EP 556585 | Neoplasm |
| Industries Ltd. | | |
| Tanabe Seiyaku Co Ltd. | JP-11071280 | Neoplasm, Lung tumor |
| University of California | U.S. Pat. No. | Neoplasm, Viral infection |
| | 5698674 | |
| University of Michigan | WO 96/34003 | Breast tumor, Lung tumor, |
| | | Prostate tumor |
| University of New Jersey | WO 97/29106 | Neoplasm, Central nervous |
| | | system disease |
| University of New Jersey-- | WO 96/36612 | Burkitts lymphoma, |
| | | Leukemia, |
| | | Myeloproliferative |
| | | disorder |
| University of Pittsburgh-- | WO 99/01456 | Malignant neoplastic |
| | | disease |
| Wisconsin Alumni | WO 96/33988 | Prostate tumor, Colon |
| Research Foundation | | tumor, Lung tumor, |
| | | Melanoma, Breast tumor, |
| | | HIV infection |
| Wisconsin Alumni | WO 97/31936 | Neoplasm |
| Research Foundation | | |
| Xenova Ltd. | WO 98/17649 | Neoplasm |
| Yale University | WO 98/40104 | Carcinoma |

Additional DNA topoisomerase I inhibiting agents of interest that can be used in the methods, combinations and compositions of the present invention are provided in Table No. 5, below. The therapeutic compounds of Table No. 5 can be used in the methods, combinations and compositions of the present invention in a variety of forms, including acid form, salt form, racemates, enantiomers, zwitterions, and tautomers.

TABLE No. 5
Additional DNA Topoisomerase I Inhibitors
Table 5: Additional DNA Topoisomerase I Inhibitors

| Compound Name | Company |
|---|---|
| BAY-38-3441 | Bayer AG |
| BNP-1350 | BioNumerik |
| GG-211 | Tigen |
| J-107088 | Merck & Co |
| kareniticin | BioNumerik Pharmaceuticals Inc |
| L9NC | MD Anderson Cancer Center |
| lurtotecan, Gilead | Gilead Sciences |
| MAG-CPT | Pharmacia |
| PEG-camptothecin, Enzon | Enzon |
| SN-22995 | University of Auckland |
| TRK-710 | Toray Industries Inc |
| NX-211 | Glaxo Wellcome plc |
| pyrazoloacridine, Wayne State | Non-industrial source |
| TAS-103 | Taiho |
| XR-5000 | Xenova |
| 9-aminocamptothecin | IDEC; Research Triangle Institute |
| rubitecan | SuperGen; Stehlin Foundation For |
| | Cancer Research |
| 10-hydroxycamptothecin | Chiba University |
| derivatives, Chiba | |
| AG-555 | Hebrew University of Jerusalem |
| anhydrous delivery system, | Matrix Pharmaceutical Inc |
| Matrix | |
| ascididemin | INSERM |
| BM-2419-1 | Kaken Pharmaceutical Co Ltd. |
| camptothecin analogs, RTI/BMS | Research Triangle Institute |
| camptothecin-TCS, Inex | Inex Pharmaceuticals Corp |
| CT-17 | University of Kentucky |
| DMNQ derivatives, Chungnam | Chungnam University |
| University | |
| DRF-1644 | Dr Reddys Research Foundation |
| dual topoisomerase I/II- | University of Auckland |
| directed anticancer drugs, | |
| University of Auckland | |
| HAR-7 | Harrier Inc |
| J-109404 | Banyu Pharmaceutical Co Ltd. |
| julibrosides | Taisho Pharmaceutical Co Ltd. |
| MPI-5019 | Matrix Pharmaceutical Inc |
| NSC-314622 | National Cancer Institute |
| NU/ICRF-505 | Imperial Cancer Research |
| | Technology Ltd. |
| NU-UB-150 | Napier University of Edinburgh |
| topoisomerase I inhibitors, | Glaxo Wellcome plc |
| Glaxo | |
| topoisomerase I inhibitors, | MediChem. Research Inc |
| MediChem/Mayo | |
| topoisomerase I inhibitors, | Purdue University |
| Purdue University/NCI | |
| topoisomerase I inhibitors, | Morphochem Inc |
| SMT | |
| topoisomerase inhibitor, | Daiichi Seiyaku Co Ltd. |
| Daiichi | |
| UCE-1022 | Kyowa Hakko Kogyo Co Ltd. |
| camptothecin, Aphios | Aphios |
| F-12167 | Pierre Fabre |
| ST-1481 | Sigma-Tau |
| topoisomerase inhibitors, | BTG |
| BTG | |
| XR-11576 | Xenova |
| gemifloxacin mesylate | LG Chemical |
| BN-80245 | Institut Henri Beaufour |

Specific DNA topoisomerase I inhibiting agents of interest that can be used in the methods, combinations and compositions of the present invention include irinotecan; irinotecan hydrochloride; camptothecin; 9-aminocamptothecin; 9-nitrocamptothecin; 9-chloro-10-hydroxy camptothecin; topotecan; topotecan hydrochloride; lurtotecan; lurtotecan dihydrochloride; lurtotecan (liposomal); homosilatecans; 6,8-dibromo-2-methyl-3-[2-(D-xylopyranosylamino)phenyl]-4(3H)-quinazolinone; 2-cyano-3-(3,4-dihydroxyphenyl)-N-(phenylmethyl)-(2E)-2-propenamide; 2-cyano-3-(3,4-dihydroxyphenyl)-N-(3-hydroxyphenylpropyl)-(E)-2-propenamide; 5H-indolo[2,3-a]pyrrolo[3,4-c]carbazole-5,7(6H)-dione, 12-.beta.-D-glucopyranosyl-12,13-dihydro-2,10-dihydroxy-6-[[2-hydroxy-1-(hydroxymethyl)ethyl]amino]-; 4-acridinecarboxamide, N-[2-(dimethylamino)ethyl]-, dihydrochloride; and 4-acridinecarboxamide, N-[2-(dimethylamino)ethyl]-.

Included in the methods, combinations and compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Also included in the methods, combinations and compositions of the present invention are the prodrugs of the described compounds and the pharmaceutically-acceptable salts thereof. The term “prodrug” refers to compounds which are drug precursors which, following administration to a subject and subsequent absorption, are converted to an active species in vivo via some process, such as a metabolic process. Other products from the conversion process are easily disposed of by the body. More preferred prodrugs produce products from the conversion process which are generally accepted as safe. Nonlimiting examples of “prodrugs” that can be used in the methods, combinations and compositions of the present invention include parecoxib (propanamide, N-[[4-(5-methyl-3-phenyl-4-isoxazolyl)phenyl]sulfonyl]-), and MAG-camptothecin.

In one embodiment, the methods, combinations and compositions of the present invention can be useful for the treatment or prevention of a neoplasia disorder selected from acral lentiginous melanoma, an actinic keratosis, adenocarcinoma, adenoid cycstic carcinoma, an adenoma, adenosarcoma, adenosquamous carcinoma, an astrocytic tumor, bartholin gland carcinoma, basal cell carcinoma, a bronchial gland carcinoma, capillary carcinoma, a carcinoid, carcinoma, carcinosarcoma, cavernous carcinoma, cholangiocarcinoma, chondosarcoma, choriod plexus papilloma, choriod plexus carcinoma, clear cell carcinoma, cystadenoma, endodermal sinus tumor, endometrial hyperplasia, endometrial stromal sarcoma, endometrioid adenocarcinoma, ependymal carcinoma, epitheloid carcinoma, Ewing's sarcoma, fibrolamellar, focal nodular hyperplasia, gastrinoma, a germ cell tumor, glioblastoma, glucagonoma, hemangiblastoma, hemangioendothelioma, a hemangioma, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, a lentigo maligna melanoma, malignant melanoma, a malignant mesothelial tumor, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, a pituitary tumor, plasmacytoma, pseudosarcoma, pulmonary blastoma, renal cell carcinoma, retinoblastoma, rhabdomyosarcoma, sarcoma, serous carcinoma, small cell carcinoma, a soft tissue carcinoma, somatostatin-secreting tumor, squamous carcinoma, squamous cell carcinoma, submesothelial, superficial spreading melanoma, undifferentiated carcinoma, uveal melanoma, verrucous carcinoma, vipoma, a well differentiated carcinoma, and Wilm's tumor.

In another embodiment, the methods, combinations and compositions of the present invention can be useful for the treatment or prevention of a neoplasia disorder where the neoplasia disorder is located in a tissue of the mammal. The tissues where the neoplasia disorder may be located include the lung, breast, skin, stomach, intestine, esophagus, bladder, head, neck, brain, cervical, or ovary of the mammal.

The phrase “neoplasia disorder effective” is intended to qualify the amount of each agent that will achieve the goal of improvement in neoplastic disease severity and the frequency of a neoplastic disease event over treatment of each agent by itself, while avoiding adverse side effects typically associated with alternative therapies.

A “neoplasia disorder effect” or “neoplasia disorder effective amount” is intended to qualify the amount of a selective Cox-2 inhibiting agent and a DNA topoisomerase I inhibiting agent required to treat or prevent a neoplasia disorder or relieve to some extent or one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent one or more of the symptoms associated with the disorder; and/or 7) relieving or reducing the side effects associated with the administration of anticancer agents.

The phrase “combination therapy” (or “co-therapy”) embraces the administration of a selective Cox-2 inhibiting agent and a DNA topoisomerase I inhibiting agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, an antineoplastic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

“Therapeutic compound” means a compound useful in the prophylaxis or treatment of a neoplastic disease.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “prevention,” in relation to neoplasia, tumor growth or tumor cell growth, means no tumor or tumor cell growth if none had occurred, no further tumor or tumor cell growth if there had already been growth.

The term “chemoprevention” refers to the use of agents to arrest or reverse the chronic cancer disease process in its earliest stages before it reaches its terminal invasive and metastatic phase.

The term “clinical tumor” includes neoplasms that are identifiable through clinical screening or diagnostic procedures including, but not limited to, palpation, biopsy, cell proliferation index, endoscopy, mammagraphy, digital mammography, ultrasonography, computed tomagraphy (CT), magnetic resonance imaging (MRI), positron emission tomagraphy (PET), radiography, radionuclide evaluation, CT- or MRI-guided aspiration cytology, and imaging-guided needle biopsy, among others. Such diagnostic techniques are well known to those skilled in the art and are described in Cancer Medicine 4^{th} Edition, Volume One. J. F. Holland, R. C. Bast, D. L. Morton, E. Frei III, D. W. Kufe, and R. R. Weichselbaum (Editors). Williams & Wilkins, Baltimore (1997).

The term “angiogenesis” refers to the process by which tumor cells trigger abnormal blood vessel growth to create their own blood supply. Angiogenesis is believed to be the mechanism via which tumors get needed nutrients to grow and metastasize to other locations in the body. Antiangiogenic agents interfere with these processes and destroy or control tumors. Angiogenesis an attractive therapeutic target for treating neoplastic disease because it is a multi-step process that occurs in a specific sequence, thus providing several possible targets for drug action. Examples of agents that interfere with several of these steps include compounds such as matrix metalloproteinase inhibitors (MMPIs) that block the actions of enzymes that clear and create paths for newly forming blood vessels to follow; compounds, such as αvβ3 inhibitors, that interfere with molecules that blood vessel cells use to bridge between a parent blood vessel and a tumor; agents, such as selective Cox-2 inhibiting agents, that prevent the growth of cells that form new blood vessels; and protein-based compounds that simultaneously interfere with several of these targets.

The present invention also provides a method for lowering the risk of a first or subsequent occurrence of a neoplastic disease event comprising the administration of a prophylactically effective amount of a combination of a DNA topoisomerase I inhibiting agent and a selective Cox-2 inhibiting agent to a patient at risk for such a neoplastic disease event. The patient may already have non-malignant neoplastic disease at the time of administration, or be at risk for developing it.

Patients to be treated with the present combination therapy includes those at risk of developing neoplastic disease or of having a neoplastic disease event. Standard neoplastic disease risk factors are known to the average physician practicing in the relevant field of medicine. Such known risk factors include but are not limited to genetic factors and exposure to carcinogens such as certain viruses, certain chemicals, tobacco smoke or radiation. Patients who are identified as having one or more risk factors known in the art to be at risk of developing neoplastic disease, as well as people who already have neoplastic disease, are intended to be included within the group of people considered to be at risk for having a neoplastic disease event.

Studies indicate that prostaglandins synthesized by cyclooxygenases play a critical role in the initiation and promotion of cancer. Moreover, Cox-2 is overexpressed in neoplastic lesions of the colon, breast, lung, prostate, esophagus, pancreas, intestine, cervix, ovaries, urinary bladder, and head and neck. Products of Cox-2 activity, i.e., prostaglandins, stimulate proliferation, increase invasiveness of malignant cells, and enhance the production of vascular endothelial growth factor, which promotes angiogenesis. In several in vitro and animal models, Cox-2 selective inhibiting agents have inhibited tumor growth and metastasis. The utility of Cox-2 selective inhibiting agents as chemopreventive, antiangiogenic and chemotherapeutic agents is described in the literature, see for example Koki et al., Potential utility of Cox-2 selective inhibiting agents in chemoprevention and chemotherapy. Exp. Opin. Invest. Drugs (1999) 8(10) pp. 1623-1638.

In addition to cancers per se, Cox-2 is also expressed in the angiogenic vasculature within and adjacent to hyperplastic and neoplastic lesions indicating that Cox-2 plays a role in angiogenesis. In both the mouse and rat, Cox-2 selective inhibiting agents markedly inhibited bFGF-induced neovascularization.

Also, Cox-2 levels are elevated in tumors with amplification and/or overexpression of other oncogenes including but not limited to c-myc, N-myc, L-myc, K-ras, H-ras, N-ras. Consequently, the administration of a selective Cox-2 inhibiting agent and a DNA topoisomerase I inhibitor, in combination with an agent, or agents, that inhibits or suppresses oncogenes is contemplated to prevent or treat cancers in which oncogenes are overexpressed.

Accordingly, there is a need for a method of treating or preventing a cancer in a patient that overexpresses Cox-2 and/or an oncogene.

Dosage of a Selective Cox-2 Inhibiting Agent and DNA Topoisomerase Inhibiting Agent

Dosage levels of the source of a Cox-2 inhibiting agent (e.g., a Cox-2 selective inhibiting agent or a prodrug of a Cox-2 selective inhibiting agent) on the order of about 0.1 mg to about 10,000 mg of the active antiangiogenic ingredient compound are useful in the treatment of the above conditions, with preferred levels of about 1.0 mg to about 1,000 mg. The amount of active ingredient that may be combined with other anticancer agents to produce a single dosage form will vary depending upon the host treated and the particular mode of administration.

A total daily dose of a DNA topoisomerase I inhibiting agent can generally be in the range of from about 0.001 to about 10,000 mg/day in single or divided doses.

It is understood, however, that specific dose levels of the therapeutic agents or therapeutic approaches of the present invention for any particular patient depends upon a variety of factors including the activity of the specific compound employed, the age, body weight, general health, sex, and diet of the patient, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of cancers in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where an compound is found to demonstrate in vitro activity at, e.g., 10 μM, one will desire to administer an amount of the drug that is effective to provide about a 10 μM concentration in vivo. Determination of these parameters are well within the skill of the art.

These considerations, as well as effective formulations and administration procedures are well known in the art and are described in standard textbooks.

Dosages, Formulations, and Routes of Administration

The Cox-2 selective inhibiting agents and/or DNA topoisomerase I inhibiting agents can be formulated as a single pharmaceutical composition or as independent multiple pharmaceutical compositions. Pharmaceutical compositions according to the present invention include those suitable for oral, inhalation spray, rectal, topical, buccal (e.g., sublingual), or parenteral (e.g., subcutaneous, intramuscular, intravenous, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

Compounds and composition of the present invention can then be administered orally, by inhalation spray, rectally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

The compositions of the present invention can be administered for the prophylaxis or treatment of neoplastic disease or disorders by any means that produce contact of these compounds with their site of action in the body, for example in the ileum, the plasma, or the liver of a mammal.

Pharmaceutically acceptable salts are particularly suitable for medical applications because of their greater aqueous solubility relative to the parent compound. Such salts must clearly have a pharmaceutically acceptable anion or cation. The anions useful in the methods, combinations and compositions of the present invention are, of course, also required to be pharmaceutically acceptable and are also selected from the above list.

The compounds useful in the methods, combinations and compositions of the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well known techniques of pharmacy, consisting essentially of admixing the components.

The amount of compound in combination that is required to achieve the desired biological effect will, of course, depend on a number of factors such as the specific compound chosen, the use for which it is intended, the mode of administration, and the clinical condition of the recipient.

The compounds of the present invention can be delivered orally either in a solid, in a semi-solid, or in a liquid form. Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a DNA topoisomerase I inhibitor, the dose range may be from about 0.01 mg to about 5,000 mg or any other dose, dependent upon the specific inhibitor, as is known in the art. When in a liquid or in a semi-solid form, the combinations of the present invention can, for example, be in the form of a liquid, syrup, or contained in a gel capsule (e.g., a gel cap). In one embodiment, when a DNA topoisomerase I inhibiting agent is used in a combination of the present invention, the DNA topoisomerase I inhibiting agent can be provided in the form of a liquid, syrup, or contained in a gel capsule. In another embodiment, when a Cox-2 selective inhibiting agent is used in a combination of the present invention, the Cox-2 selective inhibiting agent can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Pharmaceutical compositions suitable for rectal administration are preferably presented as unit-dose suppositories. These can be prepared by admixing a compound or compounds of the present invention with one or more conventional solid carriers, for example, cocoa butter, synthetic mono- di- or triglycerides, fatty acids and polyethylene glycols that are solid at ordinary temperatures but liquid at the rectal temperature and will therefore melt in the rectum and release the drug; and then shaping the resulting mixture.

Pharmaceutical compositions suitable for topical application to the skin preferably take the form of an ointment, cream, lotion, paste, gel, spray, aerosol, or oil. Carriers which can be used include petroleum jelly (e.g., Vaseline), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research, 3(6), 318 (1986).

In any case, the amount of active ingredients that can be combined with carrier materials to produce a single dosage form to be administered will vary depending upon the host treated and the particular mode of administration.

In combination therapy, administration of two or more of the therapeutic agents useful in the methods, combinations and compositions of the present invention may take place sequentially in separate formulations, or may be accomplished by simultaneous administration in a single formulation or in a separate formulation. Independent administration of each therapeutic agent may be accomplished by, for example, oral, inhalation spray, rectal, topical, buccal (e.g., sublingual), or parenteral (e.g., subcutaneous, intramuscular, intravenous, intramedullary and intradermal injections, or infusion techniques) administration. The formulation may be in the form of a bolus, or in the form of aqueous or non-aqueous isotonic sterile injection solutions or suspensions. Solutions and suspensions may be prepared from sterile powders or granules having one or more pharmaceutically-acceptable carriers or diluents, or a binder such as gelatin or hydroxypropylmethyl cellulose, together with one or more of a lubricant, preservative, surface active or dispersing agent. The therapeutic compounds may further be administered by any combination of, for example, oral/oral, oral/parenteral, or parenteral/parenteral route.

The therapeutic compounds which make up the combination therapy may be a combined dosage form or in separate dosage forms intended for substantially simultaneous oral administration. The therapeutic compounds which make up the combination therapy may also be administered sequentially, with either therapeutic compound being administered by a regimen calling for two step ingestion. Thus, a regimen may call for sequential administration of the therapeutic compounds with spaced-apart ingestion of the separate, active agents. The time period between the multiple ingestion steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

Administration Regimen

Any effective treatment regimen can be utilized and readily determined and repeated as necessary to effect treatment. In clinical practice, the compositions containing a Cox-2 selective inhibiting agent in combination with a DNA topoisomerase I inhibiting agent, (along with other therapeutic agents) are administered in specific cycles until a response is obtained.

For patients who initially present without advanced or metastatic cancer, a Cox-2 selective inhibiting agent based drug in combination with a DNA topoisomerase I inhibiting agent can be used as an immediate initial therapy prior to surgery, chemotherapy, or radiation therapy, and/or as a continuous post-treatment therapy in patients at risk for recurrence or metastasis (for example, in adenocarcinoma of the prostate, risk for metastasis is based upon high PSA, high Gleason's score, locally extensive disease, and/or pathological evidence of tumor invasion in the surgical specimen). The goal in these patients is to inhibit the growth of potentially metastatic cells from the primary tumor during surgery or radiotherapy and inhibit the growth of tumor cells from undetectable residual primary tumor.

For patients who initially present with advanced or metastatic cancer, a Cox-2 selective inhibiting agent based drug in combination with a DNA topoisomerase I inhibiting agent is used as a continuous supplement to, or possible replacement for chemotherapeutic regimes. The goal in these patients is to slow or prevent tumor cell growth from both the untreated primary tumor and from the existing metastatic lesions.

In addition, the invention may be particularly efficacious during post-surgical recovery, where the present compositions and methods may be particularly effective in lessening the chances of recurrence of a tumor engendered by shed cells that cannot be removed by surgical intervention.

Combinations with Other Treatments

The methods, combinations and compositions of the present invention may be used in conjunction with other cancer treatment modalities, including, but not limited to surgery and radiation, hormonal therapy, immunotherapy, cryotherapy, chemotherapy and antiangiogenic therapy. The present invention may be used in conjunction with any current or future therapy. The following discussion highlights some agents in this respect, which are illustrative, not limitative. A wide variety of other effective agents also may be used.

Surgery and Radiation

In general, surgery and radiation therapy are employed as potentially curative therapies for patients under 70 years of age who present with clinically localized disease and are expected to live at least 10 years. For example, approximately 70% of newly diagnosed prostate cancer patients fall into this category. Approximately 90% of these patients (65% of total patients) undergo surgery, while approximately 10% of these patients (7% of total patients) undergo radiation therapy. Histopathological examination of surgical specimens reveals that approximately 63% of patients undergoing surgery (40% of total patients) have locally extensive tumors or regional (lymph node) metastasis that was undetected at initial diagnosis. These patients are at a significantly greater risk of recurrence. Approximately 40% of these patients will actually develop recurrence within five years after surgery. Results after radiation are even less encouraging. Approximately 80% of patients who have undergone radiation as their primary therapy have disease persistence or develop recurrence or metastasis within five years after treatment. Currently, most of these surgical and radiotherapy patients generally do not receive any immediate follow-up therapy. Rather, they are monitored frequently for elevated Prostate Specific Antigen (“PSA”), which is the primary indicator of recurrence or metastasis.

Thus, there is considerable opportunity to use the present invention in conjunction with surgical intervention or radiotherapy to inhibit the growth of potentially metastatic cells from the primary tumor, as well as to inhibit the growth of tumor cells from undetectable residual primary tumor. In addition, the invention may be particularly efficacious during post-surgical recovery, where the present compositions and methods may be particularly effective in lessening the chances of recurrence of a tumor engendered by shed cells that cannot be removed by surgical intervention.

Hormonal Therapy

Hormonal ablation is the most effective palliative treatment for the 10% of patients presenting with metastatic prostate cancer at initial diagnosis. Hormonal ablation by medication and/or orchiectomy is used to block hormones that support the further growth and metastasis of prostate cancer. With time, both the primary and metastatic tumors of virtually all of these patients become hormone-independent and resistant to therapy. Approximately 50% of patients presenting with metastatic disease die within three years after initial diagnosis, and 75% of such patients die within five years after diagnosis. Continuous supplementation with NAALADase inhibitor based drugs are used to prevent or reverse this potentially metastasis-permissive state.

Suitable hormonal-type antineoplastic agents that may be used in the methods, combinations and compositions of the present invention include, but are not limited to Abarelix; Abbott A-84861; Abiraterone acetate; Aminoglutethimide; anastrozole; Asta Medica AN-207; Antide; Chugai AG-041 R; Avorelin; aseranox; Sensus B2036-PEG; Bicalutamide; buserelin; BTG CB-7598; BTG CB-7630; Casodex; cetrolix; clastroban; clodronate disodium; Cosudex; Rotta Research CR-1505; cytadren; crinone; deslorelin; droloxifene; dutasteride; Elimina; Laval University EM-800; Laval University EM-652; epitiostanol; epristeride; Mediolanum EP 23904; EntreMed 2-ME; exemestane; fadrozole; finasteride; flutamide; formestane; Pharmacia & Upjohn FCE-24304; ganirelix; goserelin; Shire gonadorelin agonist; Glaxo Wellcome GW-5638; Hoechst Marion Roussel Hoe-766; NCI hCG; idoxifene; isocordoin; Zeneca ICI-182780; Zeneca ICI-118630; Tulane University J015X; Schering Ag J96; ketanserin; lanreotide; Milkhaus LDI-200; letrozol; leuprolide; leuprorelin; liarozole; lisuride hydrogen maleate; loxiglumide; mepitiostane; Leuprorelin; Ligand Pharmaceuticals LG-1127; LG-1447; LG-2293; LG-2527; LG-2716; Bone Care International LR-103; Lilly LY-326315; Lilly LY-353381-HCl; Lilly LY-326391; Lilly LY-353381; Lilly LY-357489; miproxifene phosphate; Orion Pharma MPV-2213ad; Tulane University MZ-4-71; nafarelin; nilutamide; Snow Brand NKS01; octreotide; Azko Nobel ORG-31710; Azko Nobel ORG-31806; orimeten; orimetene; orimetine; ormeloxifene; osaterone; Smithkline Beecham SKB-105657; Tokyo University OSW-1; Peptech PTL-03001; Pharmacia & Upjohn PNU-156765; quinagolide; ramorelix; Raloxifene; statin; sandostatin LAR; Shionogi S-10364; Novartis SMT-487; somavert; somatostatin; tamoxifen; tamoxifen methiodide; teverelix; toremifene; triptorelin; TT-232; vapreotide; vorozole; Yamanouchi YM-116; Yamanouchi YM-511; Yamanouchi YM-55208; Yamanouchi YM-53789; Schering AG ZK-1911703; Schering AG ZK-230211; and Zeneca ZD-182780.

In one embodiment, some hormonal agents that may be used in the methods, combinations and compositions of the present invention include, but are not limited to, those identified in Table No. 6, below.

TABLE No. 6
Hormonal agents

| | Common Name/ | | | |
|---|---|---|---|---|
| Compound | Trade Name | Company | Reference | Dosage |
| 2-methoxyestradiol | EntreMed; 2-ME | EntreMed | | |
| N-(S)-tetrahydrofuroyl-Gly-D2Nal- | A-84861 | Abbott | | |
| D4CIPhe-D3Pal-Ser-NMeTyr- | | | | |
| DLys(Nic)-Leu-Lys(lsp)-Pro-DAla- | | | | |
| NH2 | | | | |
| | raloxifene | | | |
| [3R-1-(2,2-Dimethoxyethyl)-3-((4- | AG-041R | Chugai | WO 94/19322 | |
| methylphenyl)aminocarbonylmethyl)- | | | | |
| 3-(N′-(4-me thylphenyl)ureido)- | | | | |
| indoline-2-one] | | | | |
| | AN-207 | Asta Medica | WO 97/19954 | |
| Ethanamine, 2-[4-(4-chloro-1,2- | toremifene; | Orion Pharma | EP 95875 | 60 mg/d |
| diphenyl-1-butenyl)phenoxy]-N,N- | Fareston ® | | | |
| dimethyl-, (Z)- | | | | |
| Ethanamine, 2-[4-(1,2-diphenyl-1- | tamoxifen | Zeneca | U.S. Pat. No. | For patients |
| butenyl)phenoxy]-N,N-dimethyl-,(Z)- | Nolvadex ® | | 4536516 | with breast |
| | | | | cancer, the |
| | | | | recommended |
| | | | | daily dose is |
| | | | | 20-40 mg. |
| | | | | Dosages greater |
| | | | | than |
| | | | | 20 mg per day |
| | | | | should be |
| | | | | divided |
| | | | | (morning and |
| | | | | evening). |
| D-Alaninamide N-acetyl-3-(2- | Antide; ORF-23541 | Ares-Serono | WO 89/01944 | 25 or 50 microg/ |
| naphthalenyl)-D-alanyl-4-chloro-D- | | | | kg sc |
| phenylalanyl-3-(3 -pyridinyl)-D- | | | | |
| alanyl-L-seryl-N6-(3- | | | | |
| pyridinylcarbonyl)-L-lysyl-N6-(3- | | | | |
| pyridinylca rbonyl)-D-lysyl-L-leucyl- | | | | |
| N6-(1-methylethyl)-L-lysyl-L-prolyl- | | | | |
| | B2036-PEG; | Sensus | | |
| | Somaver; Trovert | | | |
| 4-Methyl-2-[4-[2-(1- | EM-800; EM-652 | Laval University | | |
| piperidinyl)ethoxy]phenyl]-7- | | | | |
| (pivaloyloxy)-3-[4-(pivaloyloxy) | | | | |
| phenyl]-2H-1-benzopyran | | | | |
| | letrozol | | U.S. Pat. No. | |
| | | | 4749346 | |
| | goserelin | | U.S. Pat. No. | |
| | | | 4100274 | |
| 3-[4-[1,2-Diphenyl-1(Z)- | GW-5638 | Glaxo Wellcome | | |
| butenyl]phenyl]-2(E)-propenoic | | | | |
| acid | | | | |
| Estra-1,3,5(10)-triene-3,17-diol, 7- | ICI-182780; | Zeneca | EP 34/6014 | 250 mg/mth |
| [9-[(4,4,5,5,5-pentafluoro-pentyl) | Faslodex; ZD- | | | |
| sulfinyl]-nonyl]-, (7alpha, 17beta)- | 182780 | | | |
| | J015X | Tulane University | | |
| | LG-1127; LG-1447 | Ligand | | |
| | | Pharmaceuticals | | |
| | LG-2293 | Ligand | | |
| | | Pharmaceuticals | | |
| | LG-2527;LG-2716 | Ligand | | |
| | | Pharmaceuticals | | |
| | buserelin, Peptech; | Peptech | | |
| | deslorelin, Peptech; | | | |
| | PTL-03001; | | | |
| | triptorelin, Peptech | | | |
| | LR-103 | Bone Care | | |
| | | International | | |
| [2-(4-Hydroxyphenyl)-6- | LY-326315 | Lilly | WO 9609039 | |
| hydroxynaphthalen-1-yl] [4-[2-(1- | | | | |
| piperdinyl)ethoxy]pheny l]methane | | | | |
| hydrochloride | | | | |
| | LY-353381-HCI | Lilly | | |
| | LY-326391 | Lilly | | |
| | LY-353381 | Lilly | | |
| | LY-357489 | Lilly | | |
| | MPV-2213ad | Orion Pharma | EP 476944 | 0.3-300 mg |
| Isobutyryl-Tyr-D-Arg-Asp-Ala-lle- | MZ-4-71 | Tulane University | | |
| (4-Cl)-Phe-Thr-Asn-Ser-Tyr-Arg- | | | | |
| Lys-Val-Leu-(2- aminobutyryl)-Gln- | | | | |
| Leu-Ser-Ala-Arg-Lys-Leu-Leu-Gln- | | | | |
| Asp-lle-Nle-Ser 4- | | | | |
| guanidinobutylamide | | | | |
| Androst-4-ene-3,6,17-trione, 14- | NKS01; 14alpha- | Snow Brand | EP 300062 | |
| hydroxy- | OHAT; 14OHAT | | | |
| 3beta, 16beta, 17alpha- | OSW-1 | | | |
| trihydroxycholest-5-en-22-one-16- | | | | |
| O-(2-0-4-methoxybenzoyl-beta-D- | | | | |
| xy lopyranosyl)-(1-3) (2-0-acetyl- | | | | |
| alpha-L-arabinopyranoside) | | | | |
| Spiro[estra-4,9-diene-17,2′(3′H)- | Org-31710; Org- | Akzo Nobel | EP 289073 | |
| furan]-3-one, 11-[4- | 31806 | | | |
| (dimethylamino)phenyl] -4′,5′- | | | | |
| dihydro-6-methyl-, | | | | |
| (6beta,11beta,17beta)- | | | | |
| (22RS)-N-(1,1,1-trifluoro-2- | PNU-156765; FCE- | Pharmacia & | | |
| phenylprop-2-yl)-3-oxo-4-aza- | 28260 | Upjohn | | |
| 5alpha-androst-1-ene-17beta - | | | | |
| carboxamide | | | | |
| 1 -[(benzofuran-2yl)-4- | | Menarini | | |
| chlorophenylmethyl]imidazole | | | | |
| Tryptamine derivatives | | Rhone-Poulenc | WO 96/35686 | |
| | | Rorer | | |
| Permanently ionic derivatives of | | Pharmos | WO 95/26720 | |
| steroid hormones and their | | | | |
| antagonists | | | | |
| Novel tetrahydronaphthofuranone | | Meiji Seika | WO 97/30040 | |
| derivatives | | | | |
| | SMT-487; 90Y- | Novartis | | |
| | octreotide | | | |
| D-Phe-Cys-Tyr-D-Trp-Lys-Cys- | TT-232 | | | |
| Thr-NH2 | | | | |
| 2-(1H-imidazol-4-ylmethyl)-9H- | YM-116 | Yamanou-chi | | |
| carbazole monohydrochloride | | | | |
| monohydrate | | | | |
| 4-[N-(4-bromobenzyl)-N-(4- | YM-511 | Yamanou-chi | | |
| cyanophenyl)amino]-4H-1,2,4- | | | | |
| triazole | | | | |
| 2-(1H-imidazol-4-ylmethyl)-9H- | YM-55208; YM- | Yamanou-chi | | |
| carbazole monohydrochloride | 53789 | | | |
| monohydrate | | | | |
| | ZK-1911703 | Schering AG | | |
| | ZK-230211 | Schering AG | | |
| | abarelix | Praecis | | |
| | | Pharmaceuticals | | |
| Androsta-5,16-dien-3-ol, 17-(3- | abiraterone acetate; | BTG | | |
| pyridinyl)-, acetate (ester), (3beta)- | CB-7598; CB-7630 | | | |
| 2,6-Piperidinedione, 3-(4- | aminoglutethimide; | Novartis | U.S. Pat. No. | |
| aminophenyl)-3-ethyl- | Ciba-16038; | | 3944671 | |
| | Cytadren; Elimina; | | | |
| | Orimeten; | | | |
| | Orimetene; | | | |
| | Orimetine | | | |
| 1,3- | anastroz-ole; | Zeneca | EP 296749 | 1 mg/day |
| Benzenediacetonitrile, alpha, alpha, | Arimidex; ICI- | | | |
| alpha′,alpha′-tetramethyl-5-(1H- | D1033; ZD-1033 | | | |
| 1,2,4-triazol-1-ylme thyl)- | | | | |
| 5-Oxo-L-prolyl-L-histidyl-L- | avorelin; Meterelin | Mediolanum | EP 23904 | |
| tryptophyl-L-seryl-L-tyrosyl-2- | | | | |
| methyl-D-tryptophyl- L-leucyl-L- | | | | |
| arginyl-N-ethyl-L-prolinamide | | | | |
| Propanamide, N-[4-cyano-3- | bicalutamide; | Zeneca | EP 100172 | |
| (trifluoromethyl)phenyl]-3-[(4- | Casodex; Cosudex; | | | |
| fluorophenyl) sulfonyl]-2-hydroxy- | ICI-176334 | | | |
| 2-methyl-, (+/−)- | | | | |
| Luteinizing hormone-releasing | buserelin; Hoe-766; | Hoechst Marion | GB 15/23623 | 200-600 |
| factor (pig), 6-[0-(1,1- | Profact; Receptal; | Roussel | | microg/day |
| dimethylethyl)-D-serine] -9-(N- | S-746766; | | | |
| ethyl-L-prolinamide)-10- | Suprecor; Suprecur; | | | |
| deglycinamide- | Suprefact; Suprefakt | | | |
| D-Alaninamide, N-acetyl-3-(2- | cetrorelix; SB-075; | Asta Medica | EP 29/9402 | |
| naphthalenyl)-D-alanyl-4-chloro-D- | SB-75 | | | |
| phenylalanyl-3-(3-pyridinyl)-D- | | | | |
| alanyl-L-seryl-L-tyrosyl-N5- | | | | |
| (aminocarbonyl)- D-ol-L-leucyl-L- | | | | |
| arginyl-L-prolyl- | | | | |
| Phosphonic acid, | clodronate | Schering AG | | |
| (dichloromethylene)bis-, disodium | disodium, Leiras; | | | |
| salt- | Bonefos; Clasto- | | | |
| | ban; KCO-692 | | | |
| Luteinizing hormone-releasing | deslorelin; | Roberts | U.S. Pat. No. | |
| factor (pig), 6-D-tryptophan-9-(N- | gonadorelin | | 4034082 | |
| ethyl-L-prolinamide)-10- | analogue, Roberts; | | | |
| deglycinamide- | LHRH analogue, | | | |
| | Roberts; Somagard | | | |
| Phenol, 3-[1-[4-[2- | droloxifene; FK-435; | Klinge | EP 54168 | |
| (dimethylamino)ethoxy]phenyl]-2- | K-060; K-21060E; | | | |
| phenyl-1-butenyl]-, (E)- [CA S] | RP 60850 | | | |
| 4-Azaandrost-1-ene-17- | dutasteride; GG- | Glaxo Wellcome | | |
| carboxamide, N-(2,5- | 745; GI-198745 | | | |
| bis(trifluoromethyl)phenyl)-3-oxo-, | | | | |
| (5alpha,17beta)- | | | | |
| Androstan-17-ol, 2,3-epithio-, | epitiostanol; 10275- | Shionogi | U.S. Pat. No. | |
| (2alpha,3alpha,5alpha, 17beta)- | S; | | 3230215 | |
| | epithioandrostanol; | | | |
| | S-10275; | | | |
| | Thiobrestin; Thiodrol | | | |
| Androsta-3,5-diene-3-carboxylic | epristeride; ONO- | Smith-Kline | EP 289327 | 0.4-160 |
| acid, 17-(((1,1- | 9302; SK&F- | Beecham | | mg/day |
| dimethylethyl)amino)carbonyl)- | 105657; SKB- | | | |
| (17beta)- | 105657 | | | |
| estrone 3-O-sulfamate | estrone 3-O- | | | |
| | sulfamate | | | |
| 19-Norpregna-1,3,5(10)-trien-20- | ethinyl estradiol | Schering AG | DE 1949095 | |
| yne-3,17-diol, 3-(2- | sulfonate; J96; | | | |
| propanesulfonate), (17alpha)- | Turisteron | | | |
| Androsta-1,4-diene-3,17-dione, 6- | exemestane; FCE- | Pharmacia & | DE 3622841 | 5 mg/kg |
| methylene- | 24304 | Upjohn | | |
| Benzonitrile, 4-(5,6,7,8- | fadrozole; Afema; | Novartis | EP 165904 | 1 mg po bid |
| tetrahydroimidazo[1,5-a]pyridin-5- | Arensin; CGS- | | | |
| yl)-, monohydrochloride | 16949; CGS- | | | |
| | 16949A; CGS- | | | |
| | 20287; fadrozole | | | |
| | monohydrochloride | | | |
| 4-Azaandrost-1-ene-17- | finasteride; | Merck & Co | EP 155096 | 5 mg/day |
| carboxamide, N-(1,1- | Andozac; | | | |
| dimethylethyl)-3-oxo-, | ChibroProscar; | | | |
| (5alpha,17beta)- | Finastid; MK-0906; | | | |
| | MK-906; Procure; | | | |
| | Prodel; Propecia; | | | |
| | Proscar; Proskar; | | | |
| | Prostide; YM-152 | | | |
| Propanamide, 2-methyl-N-[4-nitro- | flutamide; Drogenil; | Schering Plough | U.S. Pat. No. | |
| 3-(trifluoromethyl)phenyl]- | Euflex; Eulexin; | | 4329364 | |
| | Eulexine; Flucinom; | | | |
| | Flutamida; Fugerel; | | | |
| | NK-601; Odyne; | | | |
| | Prostogenat; Sch- | | | |
| | 13521 | | | |
| Androst-4-ene-3,17-dione, 4- | formestane; 4-HAD; | Novartis | EP 346953 | 250 or 600 |
| hydroxy- | 4-OHA; CGP-32349; | | | mg/day po |
| | CRC-82/01; Depot; | | | |
| | Lentaron | | | |
| [N-Ac-D-Nal,D-pCl-Phe,D-Pal,D- | ganirelix; Org- | Roche | EP 312052 | |
| hArg(Et)2,hArg(Et)2,D-Ala]GnRH- | 37462; RS-26306 | | | |
| | gonadorelin agonist, | Shire | | |
| | Shire | | | |
| Luteinizing hormone-releasing | goserelin; ICI- | Zeneca | U.S. Pat. No. | |
| factor (pig), 6-[O-(1,1- | 118630; Zoladex; | | 4100274 | |
| dimethylethyl)-D-serine] -10- | Zoladex LA | | | |
| deglycinamide-, 2- | | | | |
| (aminocarbonyl)hydrazide | | | | |
| | hCG; | Milkhaus | | |
| | gonadotrophin; LDI- | | | |
| | 200 | | | |
| | human chorionic | NIH | | |
| | gonadotrophin; hCG | | | |
| Pyrrolidine, 1-[2-[4-[1-(4- | idoxifene; CB-7386; | BTG | EP 260066 | |
| iodophenyl)-2-phenyl-1- | CB-7432; SB- | | | |
| butenyl]phenoxy]et hyl]-, (E)- | 223030 | | | |
| | isocordoin | Indena | | |
| 2,4(1H,3H)-Quinazolinedione, 3- | ketanserin; | Johnson & | EP 13612 | |
| [2-[4-(4-fluorobenzoyl)-1- | Aseranox; Ketensin; | Johnson | | |
| piperidinyl]ethyl]- | KJK-945; | | | |
| | ketanserine; | | | |
| | Perketan; R-41468; | | | |
| | Serefrex; Serepress; | | | |
| | Sufrexal; Taseron | | | |
| L-Threoninamide, 3-(2- | lanreotide; | Beaufour-Ipsen | EP 215171 | |
| naphthalenyl)-D-alanyl-L-cysteinyl- | Angiopeptin; BIM- | | | |
| L-tyrosyl-D- tryptophyl-L-lysyl-L- | 23014; | | | |
| valyl-L-cysteinyl-, cyclic (2-7)- | Dermopeptin; Ipstyl; | | | |
| disulfide | Somatu-line; | | | |
| | Somatuline LP | | | |
| Benzonitrile, 4,4′-(1H-1,2,4-triazol- | letrozole; CGS- | Novartis | EP 236940 | 2.5 mg/day |
| 1-ylmethylene)bis- | 20267; Femara | | | |
| Luteinizing hormone-releasing | leuprolide, Atrigel; | Atrix | | |
| factor (pig), 6-D-leucine-9-(N- | leuprolide, Atrix | | | |
| ethyl-L-prolinamid e)-10- | | | | |
| deglycinamide- | | | | |
| Luteinizing hormone-releasing | leuprorelin; Abbott- | Abbott | U.S. Pat. No. | 3.75 microg |
| factor (pig), 6-D-leucine-9-(N- | 43818; Carcinil; | | 4005063 | sc q 28 |
| ethyl-L- prolinamide)-10- | Enantone; Leuplin; | | | days |
| deglycinamide- | Lucrin; Lupron; | | | |
| | Lupron Depot; | | | |
| | leuprolide, Abbott; | | | |
| | leuprolide, Takeda; | | | |
| | leuprorelin, Takeda; | | | |
| | Procren Depot; | | | |
| | Procrin; Prostap; | | | |
| | Prostap SR; TAP- | | | |
| | 144-SR | | | |
| Luteinizing hormone-releasing | leuprorelin, DUROS; | Alza | | |
| factor (pig), 6-D-leucine-9-(N- | leuprolide, DUROS; | | | |
| ethyl-L-prolinamid e)-10- | leuprorelin | | | |
| deglycinamide- | | | | |
| 1H-Benzimidazole, 5-[(3- | liarozole; Liazal; | Johnson & | EP 260744 | 300 mg bid |
| chlorophenyl)-1H-imidazol-1- | Liazol; liarozole | Johnson | | |
| ylmethyl]- | fumarate; R-75251; | | | |
| | R-85246; Ro-85264 | | | |
| Urea, N′-[(8alpha)-9,10-didehydro- | lisuride hydrogen | VUFB | | |
| 6-methylergolin-8-yl]-N,N-diethyl-, | maleate; Cuvalit; | | | |
| (Z)-2- butenedioate (1:1) | Dopergin; | | | |
| | Dopergine; Eunal; | | | |
| | Lysenyl; Lysenyl | | | |
| | Forte; Revanil | | | |
| Pentanoic acid, 4-[(3,4- | loxiglu-mide; CR- | Rotta Research | WO 87/03869 | |
| dichlorobenzoyl)amino]-5-[(3- | 1505 | | | |
| methoxypropyl) pentylamino]-5- | | | | |
| oxo-, (+/−)- | | | | |
| Androstane, 2,3-epithio-17-[(1- | mepitio-stane; | Shionogi | U.S. Pat. No. | |
| methoxycyclopentyl)oxy]-, | S-10364; Thioderon | | 3567713 | |
| (2alpha,3alpha,5alpha,17beta) - | | | | |
| Phenol, 4-[1-[4-[2- | miproxi-fene | Taiho | WO 87/07609 | 20 mg/day |
| (dimethylamino)ethoxy]phenyl]-2- | phosphate; DP-TAT- | | | |
| [4-(1-methylethyl) phenyl]-1- | 59; TAT-59 | | | |
| butenyl]-, dihydrogen phosphate | | | | |
| (ester), (E)- | | | | |
| Luteinizing hormone-releasing | nafarelin; NAG, | Roche | EP 21/234 | |
| factor (pig), 6-[3-(2-naphthalenyl)- | Syntex; Nasanyl; | | | |
| D-alanine]- | RS-94991; RS- | | | |
| | 94991-298; Synarel; | | | |
| | Synarela; Synrelina | | | |
| 2,4-Imidazolidinedione, 5,5- | nilutamide; | Hoechst Marion | U.S. Pat. No. | |
| dimethyl-3-[4-nitro-3- | Anandron; | Roussel | 4472382 | |
| (trifluoromethyl)phenyl]- | Nilandron; | | | |
| | Notostran; RU- | | | |
| | 23908 | | | |
| | obesity gene; | Lilly | WO 96/24670 | |
| | diabetes gene; | | | |
| | leptin | | | |
| L-Cysteinamide, D-phenylalanyl-L- | octreotide; | Novartis | EP 29/579 | |
| cysteinyl-L-phenylalanyl-D- | Longasta-tina; | | | |
| tryptophyl-L- lysyl-L-threonyl-N-[2- | octreotide pamoate; | | | |
| hydroxy-1-(hydroxymethyl)propyl]-, | Sando-statin; | | | |
| cyclic (2-7)- disulfide, [R-(R*,R*)]- | Sandostatin LAR; | | | |
| | Sandosta-tina; | | | |
| | Sandosta-tine; | | | |
| | SMS-201-995 | | | |
| Pyrrolidine, 1-[2-(p-(7-methoxy- | ormeloxifene; 6720- | Central Drug | DE 2329201 | |
| 2,2-dimethyl-3-phenyl-4- | CDRI; Centron; | Research Inst. | | |
| chromanyl) phenoxy)ethyl]-, trans- | Choice-7; | | | |
| | centchroman; Saheli | | | |
| 2-Oxapregna-4,6-diene-3,20- | osaterone acetate; | Teikoku Hormone | EP 193871 | |
| dione, 17-(acetyloxy)-6-chloro- | Hipros; TZP-4238 | | | |
| Pregn-4-ene-3,20-dione | progester-one; | Columbia | | |
| | Crinone | Laboratories | | |
| Sulfamide, N,N-diethyl-N′- | quinago-lide; CV- | Novartis | EP 77754 | |
| (1,2,3,4,4a,5,10,10a-octahydro-6- | 205-502; Norprolac; | | | |
| hydroxy-1- propylbenzo[g]quinolin- | SDZ-205-502 | | | |
| 3-yl)-, (3alpha,4aalpha, 10abeta)-(+/−)- | | | | |
| L-Proline, 1-(N2-(N-(N-(N-(N-(N- | ramorelix; Hoe-013; | Hoechst Marion | EP 451791 | |
| (N-(N-acetyl-3-(2-naphthalenyl)-D- | Hoe-013C; Hoe- | Roussel | | |
| alanyl)-4-chl oro-D-phenylalanyl)- | 2013 | | | |
| D-tryptophyl)-L-seryl)-L-tyrosyl)-O- | | | | |
| (6-deoxy-alpha-L-mannopyra | | | | |
| nosyl)-D-seryl)-L-leucyl)-L-arginyl)-, | | | | |
| 2-(aminocarbonyl)hydrazide- | | | | |
| | somatosta-tin analo- | Tulane University | | |
| | gues | | | |
| Ethanamine, 2-[4-(1,2-diphenyl-1- | tamoxifen; Ceadan; | Zeneca | U.S. Pat. No. | |
| butenyl)phenoxy]-N,N-dimethyl-,(Z)- | ICI-46474; Kessar; | | 4536516 | |
| | Nolgen; Nolvadex; | | | |
| | Tafoxen; Tamofen; | | | |
| | Tamoplex; | | | |
| | Tamoxas-ta; | | | |
| | Tamoxen; Tomaxen | | | |
| | tamoxifen | Pharmos | | |
| | methiodide | | | |
| Ethanamine, 2-[4-(1,2-diphenyl-1- | tamoxifen | Douglas | | |
| butenyl)phenoxy]-N,N-dimethyl-, | | | | |
| (z)- | | | | |
| D-Alaninamide, N-acetyl-3-(2- | teverelix; Antarelix | Asta Medica | | |
| naphthalenyl)-D-alanyl-4-chloro-D- | | | | |
| pheny lalanyl-3-(3-pyridinyl)-D- | | | | |
| alanyl-L-seryl-L-tyrosyl-N6- | | | | |
| (aminocarbonyl)-D-lysyl-L -leucyl- | | | | |
| N6-(1-methylethyl)-L-lysyl-L-prolyl- | | | | |
| Ethanamine, 2-[4-(4-chloro-1 ,2- | toremifene; | Orion Pharma | EP 95875 | 60 mg po |
| diphenyl-1-butenyl)phenoxy]-N,N- | Estrimex; Fareston; | | | |
| dimethyl-, (Z)- | FC-1157; FC-1157a; | | | |
| | NK-622 | | | |
| Luteinizing hormone-releasing | triptorelin; | Debio-pharm | U.S. Pat. No. | |
| factor (pig), 6-D-tryptophan- | ARVEKAP; AY- | | 4010125 | |
| | 25650; BIM-21003; | | | |
| | BN-52104; Decap- | | | |
| | eptyl; WY-42422 | | | |
| L-Tryptophanamide, D- | vapreotide; BMY- | Debio-pharm | EP 203031 | 500 microg |
| phenylalanyl-L-cysteinyl-L-tyrosyl- | 41606; Octastatin; | | | sc tid |
| D-tryptophyl-L-lysyl- L-valyl-L- | RC-160 | | | |
| cysteinyl-, cyclic (2-7)-disulfide- | | | | |
| 1H-Benzotriazole, 6-[(4- | vorozole; R-76713; | Johnson & | EP 293978 | 2.5 mg/day |
| chlorophenyl)-1H-1,2,4-triazol-1- | R-83842; Rivizor | Johnson | | |
| ylmethyl]-1-methyl- | | | | |

Among hormones that may be used in the methods, combinations and compositions of the present inventive include, diethylstilbestrol (DES), leuprolide, flutamide, cyproterone acetate, ketoconazole and amino glutethimide are preferred.

Immunotherapy

The methods, combinations and compositions of the present invention may also be used in combination with monoclonal antibodies in treating cancer. For example monoclonal antibodies may be used in treating prostate cancer. A specific example of such an antibody includes cell membrane-specific anti-prostate antibody.

The present invention may also be used with immunotherapies based on polyclonal or monoclonal antibody-derived reagents, for instance. Monoclonal antibody-based reagents are most preferred in this regard. Such reagents are well known to persons of ordinary skill in the art. Radiolabelled monoclonal antibodies for cancer therapy, such as the recently approved use of monoclonal antibody conjugated with strontium-89, also are well known to persons of ordinary skill in the art.

Cryotherapy

Cryotherapy recently has been applied to the treatment of some cancers. Methods, combinations and compositions of the present invention also could be used in conjunction with an effective therapy of this type.

Chemotherapy

Chemotherapy includes treating a patient with agents that exert antineoplastic effects, i.e., prevent the development, maturation, or spread of neoplastic cells, directly on the tumor cell, e.g., by cytostatic or cytocidal effects, and not indirectly through mechanisms such as biological response modification. There are large numbers of antineoplastic agents available in commercial use, in clinical evaluation and in pre-clinical development that could be used in the methods, combinations and compositions of the present invention for treatment of neoplasia. For convenience of discussion, antineoplastic agents are classified into the following classes, subtypes and species:

ACE inhibitors,

alkylating agents,

angiogenesis inhibitors,

angiostatin,

anthracyclines/DNA intercalators,

anti-cancer antibiotics or antibiotic-type agents,

antimetabolites,

antimetastatic compounds,

asparaginases,

bisphosphonates,

cGMP phosphodiesterase inhibitors,

calcium carbonate,

Cox-2 inhibiting agents (e.g., Cox-2 selective inhibiting agents or prodrugs of Cox-2 selective inhibiting agents)

DHA derivatives,

endostatin,

epipodophylotoxins,

genistein,

hormonal anticancer agents,

hydrophilic bile acids (URSO),

immunomodulators or immunological agents,

integrin antagonists

interferon antagonists or agents,

MMP inhibitors,

miscellaneous antineoplastic agents,

monoclonal antibodies,

nitrosoureas,

NSAIDs,

ornithine decarboxylase inhibitors,

pBATTs,

radio/chemo sensitizers/protectors,

retinoids

selective inhibitors of proliferation and migration of endothelial cells,

selenium,

stromelysin inhibitors,

taxanes,

vaccines, and

vinca alkaloids.

The major categories that some antineoplastic agents fall into include antimetabolite agents, alkylating agents, antibiotic-type agents, immunological agents, interferon-type agents, and a category of miscellaneous antineoplastic agents. Some antineoplastic agents operate through multiple or unknown mechanisms and can thus be classified into more than one category.

A first family of antineoplastic agents which may be used in combination with the present invention consists of antimetabolite-type antineoplastic agents. Antimetabolites are typically reversible or irreversible enzyme inhibitors, or compounds that otherwise interfere with the replication, translation or transcription of nucleic acids. Suitable antimetabolite antineoplastic agents that may be used in the methods, combinations and compositions of the present invention include, but are not limited to acanthifolic acid, aminothiadiazole, anastrozole, bicalutamide, brequinar sodium, capecitabine, carmofur, Ciba-Geigy CGP-30694, cladribine, cyclopentyl cytosine, cytarabine phosphate stearate, cytarabine conjugates, cytarabine ocfosfate, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, finasteride, floxuridine, fludarabine phosphate, N-(2′-furanidyl)-5-fluorouracil, Daiichi Seiyaku FO-152, fluorouracil (5-FU), 5-FU-fibrinogen, isopropyl pyrrolizine, Lilly LY-188011, Lilly LY-264618, methobenzaprim, methotrexate, Wellcome MZPES, nafarelin, norspermidine, nolvadex, NCI NSC-127716, NCI NSC-264880, NCI NSC-39661, NCI NSC-612567, Warner-Lambert PALA, pentostatin, piritrexim, plicamycin, Asahi Chemical PL-AC, stearate; Takeda TAC-788, thioguanine, tiazofurin, Erbamont TIF, trimetrexate, tyrosine kinase inhibitors, tyrosine protein kinase inhibitors, Taiho UFT, toremifene, and uricytin.

In one embodiment, some antimetabolite agents that may be used in the methods, combinations and compositions of the present invention include, but are not limited to, those identified in Table No. 6, below.

TABLE NO. 6
Antimetabolite agents

| | Common Name/ | | | |
|---|---|---|---|---|
| Compound | Trade Name | Company | Reference | Dosage |
| 1,3- | anastrozole; Arimidex ® | Zeneca | EP 296749 | 1-mg/day |
| Benzenediacetonitrile,alpha,alpha, | | | | |
| alpha′,alpha′-tetramethyl-5- | | | | |
| (1H-1,2,4-triazol-1-ylmethyl)- | | | | |
| Propanamide, N-[4-cyano-3- | bicalutamide; Casodex ® | Zeneca | EP 100172 | 50 mg once daily |
| (trifluoromethyl)phenyl]-3-[(4- | | | | |
| fluorophenyl) sulfonyl]-2- | | | | |
| hydroxy-2-methyl-, (+/−)- | | | | |
| | capecitabine | Roche | U.S. Pat. No. 5472949 | |
| Adenosine, 2-chloro-2′-deoxy-; | cladribine; 2-CdA; | Johnson & | EP 173059 | 0.09 mg/kg/day for 7 |
| 2-chloro-2′-deoxy-(beta)-D- | Leustatin ® injection; | Johnson | | days. |
| adenosine) | Leustatin ®; Leustat ® | | | |
| | Leustatine ®; RWJ-26251; | | | |
| 2(1H)-Pyrimidinone, 4-amino-1- | cytarabine ocfosfate; ara | Yamasa Corp | EP 239015 | 100-300 mg/day for |
| [5-O- | CMP stearyl ester; C-18- | | | 2 weeks |
| [hydroxy(octadecyloxy)phosphinyl]- | PCA; cytarabine phosphate | | | |
| beta-D-arabinofuranosyl]-, | stearate; Starasid; YNK- | | | |
| monosodium salt | O1; Cytosar-U ® | | | |
| 4-Azaandrost-1-ene-17- | finasteride; Propecia ® | Merck & Co | EP 155096 | |
| carboxamide, N-(1,1- | | | | |
| dimethylethyl)-3-oxo-, | | | | |
| (5alpha,17beta)- | | | | |
| | fluorouracil (5-FU) | | U.S. Pat. No. 4336381 | |
| Fludarabine phosphate. 9H- | fludarabine phosphate; 2-F- | Southern | U.S. Pat. No. 4357324 | 25 mg/m²/d IV over a |
| Purin-6-amine, 2-fluoro-9-(5-O- | araAMP; Fludara; Fludara | Research Institute; | | period of approx- |
| phosphono-beta-D- | iv; Fludara Oral; NSC- | Berlex | | imately 30 minutes |
| arabinofuranosyl) | 312887; SH-573; SH-584; | | | daily for 5 consecutive |
| | SH-586; | | | days, commenced |
| | | | | every 28 days. |
| | gemcitabine | Eli Lily | U.S. Pat. No. 4526988 | |
| N-(4-(((2,4-diamino-6- | methotrexate iv, Hyal; HA+ | Hyal Pharma- | U.S. Pat. No. 2512572 | tropho-blastic |
| pteridinyl)methyl)methylamino)b | methotrexate, Hyal; | ceutical; American | | diseases: 15 to 30 mg/d |
| enzoyl)-L-glutamic acid | methotrexate iv, HIT | Home Products; | | orally or intra- |
| | Technolog; | Lederle | | muscularly in a five- |
| | | | | day course (repeated |
| | | | | 3 to 5 times as |
| | | | | needed) |
| Luteinizing hormone-releasing | nafarelin | Roche | EP 21234 | |
| factor (pig), 6-[3-(2- | | | | |
| naphthalenyl)-D-alanine]- | | | | |
| | pentostatin; Cl-825; DCF; | Warner-Lambert | U.S. Pat. No. 3923785 | |
| | deoxycoformycin; Nipent; | | | |
| | NSC-218321; Oncopent; | | | |
| Ethanamine, 2-[4-(4-chloro-1,2- | toremifene; Fareston ® | Orion Pharma | EP 95875 | 60 mg/d |
| diphenyl-1-butenyl)phenoxy]- | | | | |
| N,N-dimethyl-, (Z)- | | | | |

A second family of antineoplastic agents which may be used in combination with the present invention consists of alkylating-type antineoplastic agents. The alkylating agents are believed to act by alkylating and cross-linking guanine and possibly other bases in DNA, arresting cell division. Typical alkylating agents include nitrogen mustards, ethyleneimine compounds, alkyl sulfates, cisplatin, and various nitrosoureas. A disadvantage with these compounds is that they not only attack malignant cells, but also other cells which are naturally dividing, such as those of bone marrow, skin, gastro-intestinal mucosa, and fetal tissue. Suitable alkylating-type antineoplastic agents that may be used in the methods, combinations and compositions of the present invention include, but are not limited to, Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromUS tine, Tanabe Seiyaku TA-077, tauromustine, temozolomide, teroxirone, tetraplatin and trimelamol.

In one embodiment some alkylating agents that may be used in the methods, combinations and compositions of the present invention include, but are not limited to, those identified in Table No. 7, below.

TABLE NO. 7
Alkylating agents

| | Common Name/ | | | |
|---|---|---|---|---|
| Compound | Trade Name | Company | Reference | Dosage |
| Platinum, diammine[1,1- | carboplatin; | Johnson Matthey | U.S. Pat. No. 4657927. U.S. Pat. No. | 360 mg/m(squared) |
| cyclobu-tanedicarboxylato(2-)]-, | Pareplatin ® | | 4140707. | I.V. on day 1 every 4 |
| (SP-4-2)- | | | | weeks. |
| Carmustine,1,3-bis(2- | BiCNU ® | Ben Venue Labora-tories, Inc. | JAMA 1985; 253 | Preferred: 150 to |
| chloroethyl)-1-nitro-sourea | | | (11): 1590-1592. | 200 mg/m² every 6 wks. |
| | etoposide | Bristol-Myers Squibb | U.S. Pat. No. 4564675 | |
| | phosphate | | | |
| | thiotepa | | | |
| Platinum, diamminedi-chloro-, | cisplatin; | Bristol-Myers Squibb | U.S. Pat. No. 4177263 | |
| (SP-4-2)- | Plationol ®-AQ | | | |
| dacarbazine | DTIC Dome | Bayer | | 2 to 4.5 mg/kg/day |
| | | | | for 10 days; 250 mg/ |
| | | | | square meter body |
| | | | | surface/ day I.V. for |
| | | | | 5 days every 3 |
| | | | | weeks |
| ifosfamide | IFEX | Bristol-Meyers Squibb | | 4-5 g/m (square) |
| | | | | single bolus dose, or |
| | | | | 1.2-2 g/m (square) |
| | | | | I.V. over 5 days. |
| | cyclophosph-amide | | U.S. Pat. No. 4537883 | |
| cis-diaminedichloroplatinum | Platinol ® | Bristol-Myers Squibb | | 20 mg/M² IV daily |
| | Cisplatin ® | | | for a 5 day cycle. |

A third family of antineoplastic agents which may be used in methods, combinations and compositions of the present invention is the antibiotic-type antineoplastic agents. Suitable antibiotic-type antineoplastic agents that may be used in the methods, combinations and compositions of the present invention include, but are not limited to Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

In one embodiment, some antibiotic anticancer agents that may be used in the methods, combinations and compositions of the present invention include, but are not limited to, those agents identified in Table No. 8, below.

TABLE NO. 8
Antibiotic anticancer agents

| | Common Name/ | | | |
|---|---|---|---|---|
| Compound | Trade Name | Company | Reference | Dosage |
| 4-Hexenoic acid, 6-(1,3- | mycophenolate | Roche | WO | 1 to 3 gm/d |
| dihydro-4-hydroxy-6-methoxy- | mofetil | | 91/19498 | |
| 7-methyl-3-oxo-5- | | | | |
| isobenzofuranyl)-4-methyl-, 2- | | | | |
| (4-morpholinyl)ethyl ester, (E)- | | | | |
| | mitoxantrone | | U.S. Pat. No. 4310666 | |
| | doxorubicin | | U.S. Pat. No. 3590028 | |
| Mitomycin and/or mitomycin-C | Mutamycin | Bristol-Myers Squibb | | After full hematological |
| | | Oncology/Immunology | | recovery from any |
| | | | | previous chemotherapy: |
| | | | | 20 mg/m² intra-venously |
| | | | | as a single dose via a |
| | | | | functioning intravenous |
| | | | | catheter. |

A fourth family of antineoplastic agents which may be used in methods, combinations and compositions of the present invention consists of synthetic nucleosides. Several synthetic nucleosides have been identified that exhibit anticancer activity. A well known nucleoside derivative with strong anticancer activity is 5-fluorouracil (5-FU). 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation, and edema. Derivatives of 5-fluorouracil with anti-cancer activity have been described in U.S. Pat. No. 4,336,381. Further 5-FU derivatives have been described in the following patents listed in Table No. 9, hereby individually incorporated by reference herein.

TABLE NO. 9
5-Fu derivatives

| | | |
|---|---|---|
| JP 50-50383 | JP 50-50384 | JP 50-64281 |
| JP 51-146482 | JP 53-84981 | |

U.S. Pat. No. 4,000,137 discloses that the peroxidate oxidation product of inosine, adenosine, or cytidine with methanol or ethanol has activity against lymphocytic leukemia. Cytosine arabinoside (also referred to as Cytarabin, araC, and Cytosar) is a nucleoside analog of deoxycytidine that was first synthesized in 1950 and introduced into clinical medicine in 1963. It is currently an important drug in the treatment of acute myeloid leukemia. It is also active against acute lymphocytic leukemia, and to a lesser extent, is useful in chronic myelocytic leukemia and non-Hodgkin's lymphoma. The primary action of araC is inhibition of nuclear DNA synthesis. Handschumacher, R. and Cheng, Y., “Purine and Pyrimidine Antimetabolites”, Cancer Medicine, Chapter XV-1, 3rd Edition, Edited by J. Holland, et al., Lea and Febigol, publishers.

5-Azacytidine is a cytidine analog that is primarily used in the treatment of acute myelocytic leukemia and myelodysplastic syndrome.

2-Fluoroadenosine-5′-phosphate (Fludara, also referred to as FaraA) is one of the most active agents in the treatment of chronic lymphocytic leukemia. The compound acts by inhibiting DNA synthesis. Treatment of cells with F-araA is associated with the accumulation of cells at the G1/S phase boundary and in S phase; thus, it is a cell cycle S phase-specific drug. InCorp of the active metabolite, F-araATP, retards DNA chain elongation. F-araA is also a potent inhibitor of ribonucleotide reductase, the key enzyme responsible for the formation of dATP. 2-Chlorodeoxyadenosine is useful in the treatment of low grade B-cell neoplasms such as chronic lymphocytic leukemia, non-Hodgkins' lymphoma, and hairy-cell leukemia. The spectrum of activity is similar to that of Fludara. The compound inhibits DNA synthesis in growing cells and inhibits DNA repair in resting cells.

A fifth family of antineoplastic agents which may be used in methods, combinations and compositions of the present invention consists of a miscellaneous family of antineoplastic agents including, but not limited to alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, calcium carbonate, Calcet, Calci-Chew, Calci-Mix, Roxane calcium carbonate tablets, caracemide, carmethizole hydrochloride, Ajinomoto CDAF, chlorsulfaquinoxalone, Chemes CHX-2053, Chemex CHX-100, Warner-Lambert CI-921, Warner-Lambert C₁₋₉₃₇, Warner-Lambert CI-941, Warner-Lambert CI-958, clanfenur, claviridenone, ICN compound 1259, ICN compound 4711, Contracan, Cell Pathways CP-461, Yakult Honsha CPT-11, crisnatol, curaderm, cytochalasin B, cytarabine, cytocytin, Merz D-609, DABIS maleate, dacarbazine, datelliptinium, DFMO, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel, Encore Pharmaceuticals E7869, elliprabin, elliptinium acetate, Tsumura EPMTC, ergotamine, etoposide, etretinate, Eulexin®, Cell Pathways Exisulind® (sulindac sulphone or CP-246), fenretinide, Merck Research Labs Finasteride, Florical, Fujisawa FR-57704, gallium nitrate, gemcitabine, genkwadaphnin, Gerimed, Chugai GLA-43, Glaxo GR-63178, grifolan NMF-5N, hexadecylphosphocholine, Green Cross HO-221, homoharringtonine, hydroxyurea, BTG ICRF-187, ilmofosine, irinotecan, isoglutamine, isotretinoin, Otsuka JI-36, Ramot K-477, ketoconazole, Otsuak K-76COONa, Kureha Chemical K-AM, MECT Corp KI-8110, American Cyanamid L-623, leucovorin, levamisole, leukoregulin, lonidamine, Lundbeck LU-23-112, Lilly LY-186641, Materna, NCI (US) MAP, marycin, Merrel Dow MDL-27048, Medco MEDR-340, megestrol, merbarone, merocyanine derivatives, methylanilinoacridine, Molecular Genetics MGI-136, minactivin, mitonafide, mitoquidone, Monocal, mopidamol, motretinide, Zenyaku Kogyo MST-16, Mylanta, N-(retinoyl)amino acids, Nilandron; Nisshin Flour Milling N-021, N-acylated-dehydroalanines, nafazatrom, Taisho NCU-190, Nephro-Calci tablets, nocodazole derivative, Normosang, NCI NSC-145813, NCI NSC-361456, NCI NSC-604782, NCI NSC-95580, octreotide, Ono ONO-112, oquizanocine, Akzo Org-10172, paclitaxel, pancratistatin, pazelliptine, Warner-Lambert PD-1111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, retinoids, Encore Pharmaceuticals R-flurbiprofen, Sandostatin; Sapporo Breweries RBS, restrictin-P, retelliptine, retinoic acid, Rhone-Poulenc RP-49532, Rhone-Poulenc RP-56976, Scherring-Plough SC-57050, Scherring-Plough SC-57068, selenium(selenite and selenomethionine), SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, Sugen SU-101, Sugen SU-5416, Sugen SU-6668, sulindac, sulindac sulfone; superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, Yamanouchi YM-534, Zileuton, ursodeoxycholic acid, and Zanosar.

In one embodiment, some miscellaneous agents that may be used in the methods, combinations and compositions of the present invention include, but are not limited to, those identified in Table No. 10, below.

TABLE NO. 10
Miscellaneous agents

| | Common Name/ | | | |
|---|---|---|---|---|
| Compound | Trade Name | Company | Reference | Dosage |
| Flutamide; 2-methyl-N-(4-nitro- | Eulexin ® | Schering Corp | | 750 mg/d in 3 8-hr doses. |
| 3-(trifluoro-methyl)phenyl) | | | | |
| propanamide | | | | |
| | Ketoconazle | | U.S. Pat. No. 4144346 | |
| | leucovorin | | U.S. Pat. No. 4148999 | |
| | levamisole | | GB 11/20406 | |
| | megestrol | | U.S. Pat. No. 4696949 | |
| | paclitaxel | | U.S. Pat. No. 5641803 | |
| Nilutamide 5,5-dimethyl 3-(4- | Nilandron | Hoechst Marion | | A total daily dose of 300 mg |
| nitro 3-(trifluoromethyl) phenyl) | | Roussel | | for 30 days followed |
| 2,4-imidazolidinedione | | | | thereafter by three tablets |
| | | | | (50 mg each) once a day for |
| | | | | a total daily dosage of 150 mg. |
| | Vinorelbine | | EP 0010458 | |
| | vinblastine | | | |
| | vincristine | | | |
| Octreotide acetate L- | Sandostatin | Sandoz Pharmaceuticals | | s.c. or i.v. administration |
| cysteinamide, D-phenylalanyl-L- | | | | Acromegaly: 50-300 mcgm |
| cysteinyl-L-phenylalanyl-D- | | | | tid. Carcinoid tumors: 100-600 mcgm/d |
| tryptophyl-L-lysyl-L-threonyl- | | | | (mean = 300 mcgm/d) |
| NSAIDs-(2-hydroxy-1- | | | | Vipomas: 200-300 mcgm |
| (hydroxymethyl)propyl)-, cyclic- | | | | in first two weeks of |
| disulfide; (R-(R*,R*) acetate salt | | | | therapy |
| Streptozocin Streptozocin 2- | Zanosar | Pharmacia & | | i.v. 1000 mg/M2 of body |
| deoxy-2- | | Upjohn | | surface per week for two |
| (((methylnitrosamino)carbonyl)amino)- | | | | weeks. |
| alpha(and beta)-D- | | | | |
| glucopyranose) | | | | |
| Selenium | | | EP 804927 | |
| L-selenomethionine | Aces ® | J. R. Carlson | | |
| | | Laborat-ories | | |
| calcium carbonate | | | | |
| sulindac sulfone | Exisuland ® | | U.S. Pat. No. 5858694 | |
| ursodeoxycholic acid | | | U.S. Pat. No. 5843929 | |
| | Cell Pathways | | | |
| | CP-461 | | | |

Additional antineoplastic agents that may be used in the methods, combinations and compositions of the of the present invention include those described in the individual patents listed in Table No. 11 below, each of which is hereby individually incorporated by reference.

TABLE NO. 11
Antineoplastic agents

| | | | |
|---|---|---|---|
| EP 0296749 | EP 0882734 | EP 00253738 | GB 02/135425 |
| WO 09/832762 | EP 0236940 | U.S. Pat. No. 5338732 | U.S. Pat. No. 4418068 |
| U.S. Pat. No. 4692434 | U.S. Pat. No. 5464826 | U.S. Pat. No. 5061793 | EP 0702961 |
| EP 0702961 | EP 0702962 | EP 0095875 | EP 0010458 |
| EP 0321122 | U.S. Pat. No. 5041424 | JP 60019790 | WO 09/512606 |
| U.S. Pat. No. 4,808614 | U.S. Pat. No. 4526988 | CA 2128644 | U.S. Pat. No. 5455270 |
| WO 99/25344 | WO 96/27014 | U.S. Pat. No. 5695966 | DE 19547958 |
| WO 95/16693 | WO 82/03395 | U.S. Pat. No. 5789000 | U.S. Pat. No. 5902610 |
| EP 189990 | U.S. Pat. No. 4500711 | FR 24/74032 | U.S. Pat. No. 5925699 |
| WO 99/25344 | U.S. Pat. No. 4537883 | U.S. Pat. No. 4808614 | U.S. Pat. No. 5464826 |
| U.S. Pat. No. 5366734 | U.S. Pat. No. 4767628 | U.S. Pat. No. 4100274 | U.S. Pat. No. 4584305 |
| U.S. Pat. No. 4336381 | JP 5050383 | JP 5050384 | JP 5064281 |
| JP 51146482 | JP 5384981 | U.S. Pat. No. 5472949 | U.S. Pat. No. 5455270 |
| U.S. Pat. No. 4140704 | U.S. Pat. No. 4537883 | U.S. Pat. No. 4814470 | U.S. Pat. No. 3590028 |
| U.S. Pat. No. 4564675 | U.S. Pat. No. 4526988 | U.S. Pat. No. 4100274 | U.S. Pat. No. 4604463 |
| U.S. Pat. No. 4144346 | U.S. Pat. No. 4749713 | U.S. Pat. No. 4148999 | GB 11/20406 |
| U.S. Pat. No. 4696949 | U.S. Pat. No. 4310666 | U.S. Pat. No. 5641803 | U.S. Pat. No. 4418068 |
| U.S. Pat. No. 5,004758 | EP 0095875 | EP 0010458 | U.S. Pat. No. 4935437 |
| U.S. Pat. No. 4,278689 | U.S. Pat. No. 4820738 | U.S. Pat. No. 4413141 | U.S. Pat. No. 5843917 |
| U.S. Pat. No. 5,858694 | U.S. Pat. No. 4330559 | U.S. Pat. No. 5851537 | U.S. Pat. No. 4499072 |
| U.S. Pat. No. 5,217886 | WO 98/25603 | WO 98/14188 | |

Table No. 13 provides illustrative examples of median dosages for selected cancer agents that may be used in present invention. It should be noted that specific dose regimen for the chemotherapeutic agents below depends upon dosing considerations based upon a variety of factors including the type of neoplasia; the stage of the neoplasm; the age, weight, sex, and medical condition of the patient; the route of administration; the renal and hepatic function of the patient; and the particular combination employed.

TABLE NO. 13
Median dosages for selected cancer agents.

| NAME OF CHEMOTHERAPEUTIC | |
|---|---|
| AGENT | MEDIAN DOSAGE |
| Asparaginase | 10,000 units |
| Bleomycin Sulfate | 15 units |
| Carboplatin | 50-450 mg. |
| Carmustine | 100 mg. |
| Cisplatin | 10-50 mg. |
| Cladribine | 10 mg. |
| Cyclophosphamide | 100 mg.-2 gm. |
| (lyophilized) | |
| Cyclophosphamide (non- | 100 mg.-2 gm. |
| lyophilized) | |
| Cytarabine (lyophilized | 100 mg.-2 gm. |
| powder) | |
| Dacarbazine | 100 mg.-200 mg. |
| Dactinomycin | 0.5 mg. |
| Daunorubicin | 20 mg. |
| Diethylstilbestrol | 250 mg. |
| Doxorubicin | 10-150 mg. |
| Etidronate | 300 mg. |
| Etoposide | 100 mg. |
| Floxuridine | 500 mg. |
| Fludarabine Phosphate | 50 mg. |
| Fluorouraci | 500 mg.-5 gm. |
| Goserelin | 3.6 mg. |
| Granisetron Hydrochloride | 1 mg. |
| Idarubicin | 5-10 mg. |
| Ifosfamide | 1-3 gm. |
| Leucovorin Calcium | 20-350 mg. |
| Leuprolide | 3.75-7.5 mg. |
| Mechlorethamine | 10 mg. |
| Medroxyprogesterone | 1 gm. |
| Melphalan | 50 mg. |
| Methotrexate | 20 mg.-1 gm. |
| Mitomycin | 5-40 mg. |
| Mitoxantrone | 20-30 mg. |
| Ondansetron Hydrochloride | 40 mg. |
| Paclitaxel | 30 mg. |
| Pamidronate Disodium | 30-90 mg. |
| Pegaspargase | 750 units |
| Plicamycin | 2,500 mcgm. |
| Streptozocin | 1 gm. |
| Thiotepa | 15 mg. |
| Teniposide | 50 mg. |
| Vinblastine | 10 mg. |
| Vincristine | 1-5 mg. |
| Aldesleukin | 22 million units |
| Epoetin Alfa | 2,000-10,000 units |
| Filgrastim | 300-480 mcgm. |
| Immune Globulin | 500 mg.-10 gm. |
| Interferon Alpha-2a | 3-36 million units |
| Interferon Alpha-2b | 3-50 million units |
| Levamisole | 50 mg. |
| Octreotide | 1,000-5,000 mcgm. |
| Sargramostim | 250-500 mcgm. |

The anastrozole used in the therapeutic methods, combinations and compositions of the present invention can be prepared in the manner set forth in U.S. Pat. No. 4,935,437. The capecitabine used in the therapeutic methods, combinations and compositions of the present invention can be prepared in the manner set forth in U.S. Pat. No. 5,472,949. The carboplatin used in the therapeutic methods, combinations and compositions of the present invention can be prepared in the manner set forth in U.S. Pat. No. 5,455,270. The Cisplatin used in the therapeutic methods, combinations and compositions of the present invention can be prepared in the manner set forth in U.S. Pat. No. 4,140,704. The cyclophoshpamide used in the therapeutic methods, combinations and compositions of the present invention can be prepared in the manner set forth in U.S. Pat. No. 4,537,883. The eflornithine (DFMO) used in the therapeutic methods, combinations and compositions of the present invention can be prepared in the manner set forth in U.S. Pat. No. 4,413,141. The docetaxel used in the therapeutic methods, combinations and compositions of the present invention can be prepared in the manner set forth in U.S. Pat. No. 4,814,470. The doxorubicin used in the therapeutic methods, combinations and compositions of the present invention can be prepared in the manner set forth in U.S. Pat. No. 3,590,028. The etoposide used in the therapeutic methods, combinations and compositions of the present invention can be prepared in the manner set forth in U.S. Pat. No. 4,564,675. The fluorouracil used in the therapeutic methods, combinations and compositions of the present invention can be prepared in the manner set forth in U.S. Pat. No. 4,336,381. The gemcitabine used in the therapeutic methods, combinations and compositions of the present invention can be prepared in the manner set forth in U.S. Pat. No. 4,526,988. The goserelin used in the therapeutic methods, combinations and compositions of the present invention can be prepared in the manner set forth in U.S. Pat. No. 4,100,274. The irinotecan used in the therapeutic methods, combinations and compositions of the present invention can be prepared in the manner set forth in U.S. Pat. No. 4,604,463. The ketoconazole used in the therapeutic methods, combinations and compositions of the present invention can be prepared in the manner set forth in U.S. Pat. No. 4,144,346. The letrozole used in the therapeutic methods, combinations and compositions of the present invention can be prepared in the manner set forth in U.S. Pat. No. 4,749,713. The leucovorin used in the therapeutic methods, combinations and compositions of the present invention can be prepared in the manner set forth in U.S. Pat. No. 4,148,999. The levamisole used in the therapeutic methods, combinations and compositions of the present invention can be prepared in the manner set forth in GB 11/20,406. The megestrol used in the therapeutic methods, combinations and compositions of the present invention can be prepared in the manner set forth in U.S. Pat. No. 4,696,949. The mitoxantrone used in the therapeutic methods, combinations and compositions of the present invention can be prepared in the manner set forth in U.S. Pat. No. 4,310,666. The paclitaxel used in the therapeutic methods, combinations and compositions of the present invention can be prepared in the manner set forth in U.S. Pat. No. 5,641,803. The Retinoic acid used in the therapeutic methods, combinations and compositions of the present invention can be prepared in the manner set forth in U.S. Pat. No. 4,843,096. The tamoxifen used in the therapeutic methods, combinations and compositions of the present invention can be prepared in the manner set forth in U.S. Pat. No. 4,418,068. The topotecan used in the therapeutic methods, combinations and compositions of the present invention can be prepared in the manner set forth in U.S. Pat. No. 5,004,758. The toremifene used in the therapeutic methods, combinations and compositions of the present invention can be prepared in the manner set forth in EP 095,875. The vinorelbine used in the therapeutic methods, combinations and compositions of the present invention can be prepared in the manner set forth in EP 010,458. The sulindac sulfone used in the therapeutic methods, combinations and compositions of the present invention can be prepared in the manner set forth in U.S. Pat. No. 5,858,694. The selenium (selenomethionine) used in the therapeutic methods, combinations and compositions of the present invention can be prepared in the manner set forth in EP 08/04,927. The ursodeoxycholic acid used in the therapeutic methods, combinations and compositions of the present invention can be prepared in the manner set forth in WO 97/34,608. Ursodeoxycholic acid can also be prepared according to the manner set forth in EP 05/99,282. Finally, ursodeoxycholic acid can be prepared according to the manner set forth in U.S. Pat. No. 5,843,929.

In another embodiment, antineoplastic agents that may be used in the methods, combinations and compositions of the present invention include: anastrozole, calcium carbonate, capecitabine, carboplatin, cisplatin, Cell Pathways CP-461, cyclophosphamide, docetaxel, doxorubicin, etoposide, Exisulind®, fluorouracil (5-FU), fluoxymestrine, gemcitabine, goserelin, irinotecan, ketoconazole, letrozol, leucovorin, levamisole, megestrol, mitoxantrone, paclitaxel, raloxifene, retinoic acid, tamoxifen, thiotepa, topotecan, toremifene, vinorelbine, vinblastine, vincristine, selenium (selenomethionine), ursodeoxycholic acid, sulindac sulfone and eflornithine (DFMO).

The phrase “taxane” includes a family of diterpene alkaloids all of which contain a particular eight (8) member “taxane” ring structure. Taxanes such as paclitaxel prevent the normal post division breakdown of microtubules which form to pull and separate the newly duplicated chromosome pairs to opposite poles of the cell prior to cell division. In cancer cells which are rapidly dividing, taxane therapy causes the microtubules to accumulate which ultimately prevents further division of the cancer cell. Taxane therapy also affects other cell processes dependant on microtubules such as cell motility, cell shape and intracellular transport. The major adverse side-effects associated with taxane therapy can be classified into cardiac effects, neurotoxicity, haematological toxicity, and hypersensitivity reactions. (See Exp. Opin. Thera. Patents (1998) 8(5), hereby incorporated by reference). Specific adverse side-effects include neutropenia, alopecia, bradycardia, cardiac conduction defects, acute hypersensitivity reactions, neuropathy, mucositis, dermatitis, extravascular fluid accumulation, arthralgias, and myalgias. Various treatment regimens have been developed in an effort to minimize the side effects of taxane therapy, but adverse side-effects remain the limiting factor in taxane therapy.

It has been recently discovered in vitro that Cox-2 expression is elevated in cells treated with taxanes. Elevated levels of Cox-2 expression are associated with inflammation and generation of other Cox-2 derived prostaglandin side effects. Consequently, when taxane therapy is provided to a patient, the administration of a Cox-2 selective inhibiting agent is contemplated to reduce the inflammatory and other Cox-2 derived prostaglandin side effects associated with taxane therapy. It is contemplated that the of addition of a DNA topoisomerase I inhibiting agent will further improve therapy options for treating, preventing or reducing the risk of developing neoplastic disease.

Taxane derivatives have been found to be useful in treating refractory ovarian carcinoma, urothelial cancer, breast carcinoma, melanoma, non-small-cell lung carcinoma, gastric, and colon carcinomas, squamous carcinoma of the head and neck, lymphoblastic, myeloblastic leukemia, and carcinoma of the esophagus.

Paclitaxel is typically administered in a 15-420 mg/m² dose over a 6 to 24 hour infusion. For renal cell carcinoma, squamous carcinoma of head and neck, carcinoma of esophagus, small and non-small cell lung cancer, and breast cancer, paclitaxel is typically administered as a 250 mg/m² 24 hour infusion every 3 weeks. For refractory ovarian cancer paclitaxel is typically dose escalated starting at 110 mg/m². Docetaxel is typically administered in a 60-100 mg/M² i.v. over 1 hour, every three weeks. It should be noted; however, that specific dose regimen depends upon dosing considerations based upon a variety of factors including the type of neoplasia; the stage of the neoplasm; the age, weight, sex, and medical condition of the patient; the route of administration; the renal and hepatic function of the patient; and the particular agents and combination employed.

In one embodiment, paclitaxel is used in the methods, combinations and compositions of the present invention in combination with a Cox-2 selective inhibiting agent, a DNA topoisomerase I inhibiting agent and with cisplatin, cyclophosphamide, or doxorubicin for the treatment of breast cancer. In another embodiment paciltaxel is used in combination with a Cox-2 selective inhibiting agent, a DNA topoisomerase I inhibiting agent and cisplatin or carboplatin, and ifosfamide for the treatment of ovarian cancer.

In another embodiment, docetaxal is used in the methods, combinations and compositions of the present invention in combination with a Cox-2 selective inhibiting agent, a DNA topoisomerase I inhibiting agent and with cisplatin, cyclophosphamide, or doxorubicin for the treatment of ovary and breast cancer and for patients with locally advanced or metastatic breast cancer who have progressed during anthracycline based therapy.

The following references listed in Table No. 14 below, hereby individually incorporated by reference herein, describe various taxanes and taxane derivatives suitable for use in the methods, combinations and compositions of the present invention, and processes for their manufacture.

TABLE NO. 14
Taxanes and taxane derivatives

| | | | |
|---|---|---|---|
| EP 694539 | EP 683232 | EP 639577 | EP 627418 |
| EP 604910 | EP 797988 | EP 727492 | EP 767786 |
| EP 767376 | U.S. Pat. No. 5886026 | U.S. Pat. No. 5880131 | U.S. Pat. No. 5879929 |
| U.S. Pat. No. 5871979 | U.S. Pat. No. 5869680 | U.S. Pat. No. 5871979 | U.S. Pat. No. 5854278 |
| U.S. Pat. No. 5840930 | U.S. Pat. No. 5840748 | U.S. Pat. No. 5827831 | U.S. Pat. No. 5824701 |
| U.S. Pat. No. 5821363 | U.S. Pat. No. 5821263 | U.S. Pat. No. 5811292 | U.S. Pat. No. 5808113 |
| U.S. Pat. No. 5808102 | U.S. Pat. No. 5807888 | U.S. Pat. No. 5780653 | U.S. Pat. No. 5773461 |
| U.S. Pat. No. 5770745 | U.S. Pat. No. 5767282 | U.S. Pat. No. 5763628 | U.S. Pat. No. 5760252 |
| U.S. Pat. No. 5760251 | U.S. Pat. No. 5756776 | U.S. Pat. No. 5750737 | U.S. Pat. No. 5744592 |
| U.S. Pat. No. 5739362 | U.S. Pat. No. 5728850 | U.S. Pat. No. 5728725 | U.S. Pat. No. 5723634 |
| U.S. Pat. No. 5721268 | U.S. Pat. No. 5717115 | U.S. Pat. No. 5716981 | U.S. Pat. No. 5714513 |
| U.S. Pat. No. 5710287 | U.S. Pat. No. 5705508 | U.S. Pat. No. 5703247 | U.S. Pat. No. 5703117 |
| U.S. Pat. No. 5700669 | U.S. Pat. No. 5693666 | U.S. Pat. No. 5688977 | U.S. Pat. No. 5684175 |
| U.S. Pat. No. 5683715 | U.S. Pat. No. 5679807 | U.S. Pat. No. 5677462 | U.S. Pat. No. 5675025 |
| U.S. Pat. No. 5670673 | U.S. Pat. No. 5654448 | U.S. Pat. No. 5654447 | U.S. Pat. No. 5646176 |
| U.S. Pat. No. 5637732 | U.S. Pat. No. 5637484 | U.S. Pat. No. 5635531 | U.S. Pat. No. 5631278 |
| U.S. Pat. No. 5629433 | U.S. Pat. No. 5622986 | U.S. Pat. No. 5618952 | U.S. Pat. No. 5616740 |
| U.S. Pat. No. 5616739 | U.S. Pat. No. 5614645 | U.S. Pat. No. 5614549 | U.S. Pat. No. 5608102 |
| U.S. Pat. No. 5599820 | U.S. Pat. No. 5594157 | U.S. Pat. No. 5587489 | U.S. Pat. No. 5580899 |
| U.S. Pat. No. 5574156 | U.S. Pat. No. 5567614 | U.S. Pat. No. 5565478 | U.S. Pat. No. 5560872 |
| U.S. Pat. No. 5556878 | U.S. Pat. No. 5547981 | U.S. Pat. No. 5539103 | U.S. Pat. No. 5532363 |
| U.S. Pat. No. 5530020 | U.S. Pat. No. 5508447 | U.S. Pat. No. 5489601 | U.S. Pat. No. 5484809 |
| U.S. Pat. No. 5475011 | U.S. Pat. No. 5473055 | U.S. Pat. No. 5470866 | U.S. Pat. No. 5466834 |
| U.S. Pat. No. 5449790 | U.S. Pat. No. 5442065 | U.S. Pat. No. 5440056 | U.S. Pat. No. 5430160 |
| U.S. Pat. No. 5412116 | U.S. Pat. No. 5412092 | U.S. Pat. No. 5411984 | U.S. Pat. No. 5407816 |
| U.S. Pat. No. 5407674 | U.S. Pat. No. 5405972 | U.S. Pat. No. 5399726 | U.S. Pat. No. 5395850 |
| U.S. Pat. No. 5384399 | U.S. Pat. No. 5380916 | U.S. Pat. No. 5380751 | U.S. Pat. No. 5367086 |
| U.S. Pat. No. 5356928 | U.S. Pat. No. 5356927 | U.S. Pat. No. 5352806 | U.S. Pat. No. 5350866 |
| U.S. Pat. No. 5344775 | U.S. Pat. No. 5338872 | U.S. Pat. No. 5336785 | U.S. Pat. No. 5319112 |
| U.S. Pat. No. 5296506 | U.S. Pat. No. 5294737 | U.S. Pat. No. 5294637 | U.S. Pat. No. 5284865 |
| U.S. Pat. No. 5284864 | U.S. Pat. No. 5283253 | U.S. Pat. No. 5279949 | U.S. Pat. No. 5274137 |
| U.S. Pat. No. 5274124 | U.S. Pat. No. 5272171 | U.S. Pat. No. 5254703 | U.S. Pat. No. 5254580 |
| U.S. Pat. No. 5250683 | U.S. Pat. No. 5243045 | U.S. Pat. No. 5229526 | U.S. Pat. No. 5227400 |
| U.S. Pat. No. 5200534 | U.S. Pat. No. 5194635 | U.S. Pat. No. 5175,315 | U.S. Pat. No. 5136060 |
| U.S. Pat. No. 5015744 | WO 98/38862 | WO 95/24402 | WO 93/21173 |
| EP 681574 | EP 681575 | EP 568203 | EP 642503 |
| EP 667772 | EP 668762 | EP 679082 | EP 681573 |
| EP 688212 | EP 690712 | EP 690853 | EP 710223 |
| EP 534708 | EP 534709 | EP 605638 | EP 669918 |
| EP 855909 | EP 605638 | EP 428376 | EP 428376 |
| EP 534707 | EP 605637 | EP 679156 | EP 689436 |
| EP 690867 | EP 605637 | EP 690867 | EP 687260 |
| EP 690711 | EP 400971 | EP 690711 | EP 400971 |
| EP 690711 | EP 884314 | EP 568203 | EP 534706 |
| EP 428376 | EP 534707 | EP 400971 | EP 669918 |
| EP 605637 | U.S. Pat. No. 5015744 | U.S. Pat. No. 5175315 | U.S. Pat. No. 5243045 |
| U.S. Pat. No. 5283253 | U.S. Pat. No. 5250683 | U.S. Pat. No. 5254703 | U.S. Pat. No. 5274124 |
| U.S. Pat. No. 5284864 | U.S. Pat. No. 5284865 | U.S. Pat. No. 5350866 | U.S. Pat. No. 5227400 |
| U.S. Pat. No. 5229526 | U.S. Pat. No. 4876399 | U.S. Pat. No. 5136060 | U.S. Pat. No. 5336785 |
| U.S. Pat. No. 5710287 | U.S. Pat. No. 5714513 | U.S. Pat. No. 5717115 | U.S. Pat. No. 5721268 |
| U.S. Pat. No. 5723634 | U.S. Pat. No. 5728725 | U.S. Pat. No. 5728850 | U.S. Pat. No. 5739362 |
| U.S. Pat. No. 5760219 | U.S. Pat. No. 5760252 | U.S. Pat. No. 5384399 | U.S. Pat. No. 5399726 |
| U.S. Pat. No. 5405972 | U.S. Pat. No. 5430160 | U.S. Pat. No. 5466834 | U.S. Pat. No. 5489601 |
| U.S. Pat. No. 5532363 | U.S. Pat. No. 5539103 | U.S. Pat. No. 5574156 | U.S. Pat. No. 5587489 |
| U.S. Pat. No. 5618952 | U.S. Pat. No. 5637732 | U.S. Pat. No. 5654447 | U.S. Pat. No. 4942184 |
| U.S. Pat. No. 5059699 | U.S. Pat. No. 5157149 | U.S. Pat. No. 5202488 | U.S. Pat. No. 5750736 |
| U.S. Pat. No. 5202488 | U.S. Pat. No. 5549830 | U.S. Pat. No. 5281727 | U.S. Pat. No. 5019504 |
| U.S. Pat. No. 4857653 | U.S. Pat. No. 4924011 | U.S. Pat. No. 5733388 | U.S. Pat. No. 5696153 |
| WO 93/06093 | WO 93/06094 | WO 94/10996 | WO 9/10997 |
| WO 94/11362 | WO 94/15599 | WO 94/15929 | WO 94/17050 |
| WO 94/17051 | WO 94/17052 | WO 94/20088 | WO 94/20485 |
| WO 94/21250 | WO 94/21251 | WO 94/21252 | WO 94/21623 |
| WO 94/21651 | WO 95/03265 | WO 97/09979 | WO 97/42181 |
| WO 99/08986 | WO 99/09021 | WO 93/06079 | U.S. Pat. No. 5202448 |
| U.S. Pat. No. 5019504 | U.S. Pat. No. 4857653 | U.S. Pat. No. 4924011 | WO 97/15571 |
| WO 96/38138 | U.S. Pat. No. 5489589 | EP 781778 | WO 96/11683 |
| EP 639577 | EP 747385 | U.S. Pat. No. 5422364 | WO 95/11020 |
| EP 747372 | WO 96/36622 | U.S. Pat. No. 5599820 | WO 97/10234 |
| WO 96/21658 | WO 97/23472 | U.S. Pat. No. 5550261 | WO 95/20582 |
| WO 97/28156 | WO 96/14309 | WO 97/32587 | WO 96/28435 |
| WO 96/03394 | WO 95/25728 | WO 94/29288 | WO 96/00724 |
| WO 95/02400 | EP 694539 | WO 95/24402 | WO 93/10121 |
| WO 97/19086 | WO 97/20835 | WO 96/14745 | WO 96/36335 |

U.S. Pat. No. 5,019,504 describes the isolation of paclitaxel and related alkaloids from culture grown Taxus brevifolia cells. U.S. Pat. No. 5,675,025 describes methods for synthesis of Taxol®, Taxol® analogues and intermediates from baccatin III. U.S. Pat. No. 5,688,977 describes the synthesis of Docetaxel from 10-deacetyl baccatin III. U.S. Pat. No. 5,202,488 describes the conversion of partially purified taxane mixture to baccatin III. U.S. Pat. No. 5,869,680 describes the process of preparing taxane derivatives. U.S. Pat. No. 5,856,532 describes the process of the production of Taxol®. U.S. Pat. No. 5,750,737 describes the method for paclitaxel synthesis. U.S. Pat. No. 6,688,977 describes methods for docetaxel synthesis. U.S. Pat. No. 5,677,462 describes the process of preparing taxane derivatives. U.S. Pat. No. 5,594,157 describes the process of making Taxol® derivatives.

Some taxanes and taxane derivatives that may be used in the methods, combinations and compositions of the present invention are described in the patents listed in Table No. 15 below, and are hereby individually incorporated by reference herein.

TABLE NO. 15
Some preferred taxanes and taxane derivatives

| | | | |
|---|---|---|---|
| U.S. Pat. No. 5015744 | U.S. Pat. No. 5136060 | U.S. Pat. No. 5175315 | U.S. Pat. No. 5200534 |
| U.S. Pat. No. 5194635 | U.S. Pat. No. 5227400 | U.S. Pat. No. 4924012 | U.S. Pat. No. 5641803 |
| U.S. Pat. No. 5059699 | U.S. Pat. No. 5157049 | U.S. Pat. No. 4942184 | U.S. Pat. No. 4960790 |
| U.S. Pat. No. 5202488 | U.S. Pat. No. 5675025 | U.S. Pat. No. 5688977 | U.S. Pat. No. 5750736 |
| U.S. Pat. No. 5684175 | U.S. Pat. No. 5019504 | U.S. Pat. No. 4814470 | WO 95/01969 |

The phrase “retinoid” includes compounds which are natural and synthetic analogues of retinol (Vitamin A). The retinoids bind to one or more retinoic acid receptors to initiate diverse processes such as reproduction, development, bone formation, cellular proliferation and differentiation, apoptosis, hematopoiesis, immune function and vision. Retinoids are required to maintain normal differentiation and proliferation of almost all cells and have been shown to reverse/suppress carcinogenesis in a variety of in vitro and in vivo experimental models of cancer, see (Moon et al., Ch. 14 Retinoids and cancer. In The Retinoids, Vol. 2. Academic Press, Inc. 1984). Also see Roberts et al. Cellular biology and biochemistry of the retinoids. In The Retinoids, Vol. 2. Academic Press, Inc. 1984, hereby incorporated by reference), which also shows that vesanoid (tretinoid trans retinoic acid) is indicated for induction of remission in patients with acute promyelocytic leukemia (APL).

A synthetic description of retinoid compounds, hereby incorporated by reference, is described in: Dawson M I and Hobbs P D. The synthetic chemistry of retinoids: in The retinoids, 2^{nd} edition. M B Sporn, A B Roberts, and D S Goodman(eds). New York: Raven Press, 1994, pp 5-178.

Lingen et al. describe the use of retinoic acid and interferon alpha against head and neck squamous cell carcinoma (Lingen, MW et al., Retinoic acid and interferon alpha act synergistically as antiangiogenic and antitumor agents against human head and neck squamous cell carcinoma. Cancer Research 58 (23) 5551-5558 (1998), hereby incorporated by reference).

Lurlaro et al. describe the use of beta interferon and 13-cis retinoic acid to inhibit angiogenesis. (Iurlaro, M et al., Beta interferon inhibits HIV-1 Tat-induced angiogenesis: synergism with 13-cis retinoic acid. European Journal of Cancer 34 (4) 570-576 (1998), hereby incorporated by reference).

Majewski et al. describe Vitamin D3 and retinoids in the inhibition of tumor cell-induced angiogenesis. (Majewski, S et al., Vitamin D3 is a potent inhibitor of tumor cell-induced angiogenesis. J. Invest. Dermatology. Symposium Proceedings, 1 (1), 97-101 (1996), hereby incorporated by reference).

Majewski et al. describe the role of retinoids and other factors in tumor angiogenesis. (Majewski, S et al., Role of cytokines, retinoids and other factors in tumor angiogenesis. Central-European journal of Immunology 21 (4) 281-289 (1996), hereby incorporated by reference).

Bollag describes retinoids and alpha-interferon in the prevention and treatment of neoplastic disease. (Bollag W. Retinoids and alpha-interferon in the prevention and treatment of preneoplastic and neoplastic diseases. Chemotherapie Journal, (Suppl) 5 (10) 55-64 (1996), hereby incorporated by reference).

Bigg, H F et al. describe all-trans retinoic acid with basic fibroblast growth factor and epidermal growth factor to stimulate tissue inhibitor of metalloproteinases from fibroblasts. (Bigg, H F et al., All-trans-retoic acid interacts synergystically with basic fibroblast growth factor and epidermal growth factor to stimulate the production of tissue inhibitor of metalloproteinases from fibroblasts. Arch. Biochem. Biophys. 319 (1) 74-83 (1995), hereby incorporated by reference).

Nonlimiting examples of retinoids that may be used in the methods, combinations and compositions of the present invention are identified in Table No. 16 below.

TABLE NO. 16
Retinoids

| | Common Name/ | | | |
|---|---|---|---|---|
| Compound | Trade Name | Company | Reference | Dosage |
| CD-271 | Adapaline | | EP 199636 | |
| Tretinoin trans retinoic | Vesanoid | Roche Holdings | | 45 mg/M²/day as two |
| acid | | | | evenly divided doses |
| | | | | until complete remission |
| 2,4,6,8-Nonatetraenoic | etretinate isoetretin; | Roche Holdings | U.S. Pat. No. 4215215 | .25-1.5 mg/kg/day |
| acid, 9-(4-methoxy- | Ro-10-9359; Ro-13- | | | |
| 2,3,6-trimethylphenyl)- | 7652; Tegison; | | | |
| 3,7-dimethyl-, ethyl | Tigason | | | |
| ester, (all-E)- | | | | |
| Retinoic acid, 13-cis- | isotretinoin | Roche Holdings | U.S. Pat. No. 4843096 | .5 to 2 mg/kg/day |
| | Accutane; Isotrex; | | | |
| | Ro-4-3780; | | | |
| | Roaccutan; | | | |
| | Roaccutane | | | |
| | Roche Ro-40-0655 | Roche Holdings | | |
| | Roche Ro-25-6760 | Roche Holdings | | |
| | Roche Ro-25-9022 | Roche Holdings | | |
| | Roche Ro-25-9716 | Roche Holdings | | |
| Benzoic acid, 4-[[3,5- | TAC-101 | Taiho Pharmaceutical | | |
| bis(trimethylsilyl)benzoyl] | | | | |
| amino]- | | | | |
| Retinamide, N-(4- | fenretinide 4-HPR; | | | 50-400 mg/kg/day |
| hydroxyphenyl)- | HPR; McN-R-1967 | | | |
| (2E,4E,6E)-7-(3,5-Di- | LGD-1550 ALRT- | Ligand Pharma- | | 20 microg/m2/day to |
| tert-butylphenyl)-3- | 1550; ALRT-550; | ceuticas; Allergan | | 400 microg/m2/day |
| methylocta-2,4,6- | LG-1550 | USA | | administered as a |
| trienoic acid | | | | single daily oral dose |
| | Molecular Design | | U.S. Pat. No. 4885311 | |
| | MDI-101 | | | |
| | Molecular Design | | U.S. Pat. No. 4677120 | |
| | MDI-403 | | | |
| Benzoic acid, 4-(1- | bexarotene LG- | | WO 94/15901 | |
| (5,6,7,8-tetrahydro- | 1064; LG-1069; | | | |
| 3,5,5,8,8-pentamethyl- | LGD-1069; | | | |
| 2-naphthalenyl)ethenyl)- | Targretin; Targretin | | | |
| | Oral; Targretin | | | |
| | Topical Gel | | | |
| Benzoic acid, 4-(1- | bexarotene, soft gel | R P Scherer | | |
| (5,6,7,8-tetrahydro- | bexarotene, Ligand; | | | |
| 3,5,8,8-pentamethyl-2- | bexaroten | | | |
| naphthalenyl)ethen yl)- | | | | |
| (2E,4E)-3-methyl-5-[3- | | | WO 96/05165 | |
| (5,5,8,8-tetramethyl- | | | | |
| 5,6,7,8-tetrahydro- | | | | |
| naphthalen-2-yl)- | | | | |
| thiopen-2-yl]-penta-2,4- | | | | |
| dienoic acid | | | | |
| | SR-11262 F | Hoffmann-La Roche | | |
| | | Ltd. | | |
| | BMS-181162 | Bristol Myers Squibb | EP 476682 | |
| N-(4- | IIT Research | | Cancer | |
| hydroxyphenyl)retinamide | Institute | | Research 39, | |
| | | | 1339-1346 | |
| | | | (1979) | |
| | AGN-193174 | Allergan USA | WO 96/33716 | |

The following individual patent references listed in Table No. 17 below, hereby individually incorporated by reference, describe various retinoid and retinoid derivatives suitable for use in the methods, combinations and compositions of the present invention described herein, and processes for their manufacture.

TABLE No. 17
Retinoids

| | | | |
|---|---|---|---|
| U.S. Pat. No. 4215215 | U.S. Pat. No. 4885311 | U.S. Pat. No. 4677120 | U.S. Pat. No. 4105681 |
| U.S. Pat. No. 5260059 | U.S. Pat. No. 4503035 | U.S. Pat. No. 5827836 | U.S. Pat. No. 3878202 |
| U.S. Pat. No. 4843096 | WO 96/05165 | WO 97/34869 | WO 97/49704 |
| EP 19/9636 | WO 96/33716 | WO 97/24116 | WO 97/09297 |
| WO 98/36742 | WO 97/25969 | WO 96/11686 | WO 94/15901 |
| WO 97/24116 | CH 61/6134 | DE 2854354 | EP 579915 |
| U.S. Pat. No. 5547947 | EP 552624 | EP 728742 | EP 331983 |
| EP 476682 | | | |

In one embodiment, retinoids that may be used in the methods, combinations and compositions of the present invention include Accutane; Adapalene; Allergan AGN-193174; Allergan AGN-193676; Allergan AGN-193836; Allergan AGN-193109; Aronex AR-623; BMS-181162; Galderma CD-437; Eisai ER-34617; Etrinate; Fenretinide; Ligand LGD-1550; lexacalcitol; Maxia Pharmaceuticals MX-781; mofarotene; Molecular Design MDI-101; Molecular Design MDI-301; Molecular Design MDI-403; Motretinide; Eisai 4-(2-[5-(4-methyl-7-ethylbenzofuran-2-yl)pyrrolyl]) benzoic acid; Johnson & Johnson N-[4-[2-thyl-1-(1H-imidazol-1-yl)butyl]phenyl]-2-benzothiazolamine; Soriatane; Roche SR-11262; Tocoretinate; Advanced Polymer Systems trans-retinoic acid; UAB Research Foundation UAB-8; Tazorac; TopiCare; Taiho TAC-101; and Vesanoid.

cGMP phosphodiesterase inhibitors, including sulindac sulfone (Exisuland®) and CP-461 for example, are apoptosis inducers and do not inhibit the cyclooxygenase pathways. cGMP phosphodiesterase inhibitors increase apoptosis in tumor cells without arresting the normal cycle of cell division or altering the cell's expression of the p53 gene.

Ornithine decarboxylase is a key enzyme in the polyamine synthesis pathway that is elevated in most tumors and premalignant lesions. Induction of cell growth and proliferation is associated with dramatic increases in ornithine decarboxylase activity and subsequent polyamine synthesis. Further, blocking the formation of polyamines slows or arrests growth in transformed cells. Consequently, polyamines are thought to play a role in tumor growth. Difluoromethylornithine (DFMO) is a potent inhibitor of ornithine decarboxylase that has been shown to inhibit carcinogen-induced cancer development in a variety of rodent models (Meyskens et al. Development of Difluoromethylornithine (DFMO) as a chemoprevention agent. Clin. Cancer Res. 1999 May, 5(%):945-951, hereby incorporated by reference, herein). DFMO is also known as 2-difluoromethyl-2,5-diaminopentanoic acid, or 2-difluoromethyl-2,5-diaminovaleric acid, or a-(difluoromethyl) ornithine; DFMO is marketed under the tradename Elformithine®. Therefore, the use of DFMO in combination with a Cox-2 selective inhibiting agent and a DNA topoisomerase I inhibiting agent is contemplated to treat or prevent cancer, including but not limited to colon cancer or colonic polyps.

Populations with high levels of dietary calcium have been reported to be protected from colon cancer. In vivo, calcium carbonate has been shown to inhibit colon cancer via a mechanism of action independent from Cox-2 inhibition. Further, calcium carbonate is well tolerated. A combination therapy consisting of calcium carbonate, a Cox-2 selective inhibiting agent, and a DNA topoisomerase I inhibiting agent is contemplated to treat or prevent cancer, including, but not limited to, colon cancer or colonic polyps.

Several studies have focused attention on bile acids as a potential mediator of the dietary influence on colorectal cancer risk. Bile acids are important detergents for fat solubilization and digestion in the proximal intestine. Specific transprot processes in the apical domain of the terminal ileal enterocyte and basolateral domain of the hepatocyte account for the efficient conservation in the enterohepatic circulation. Only a small fraction of bile acids enter the colon; however, perturbations of the cycling rate of bile acids by diet (e.g. fat) or surgery may increase the fecal bile load and perhaps account for the associated increased risk of colon cancer. (Hill M J, Bile flow and colon cancer. 238 Mutation Review, 313 (1990). Ursodeoxycholate (URSO), the hydrophilic 7-beta epimer of chenodeoxycholate, is non cytotoxic in a variety of cell model systems including colonic epithelia. URSO is also virtually free of side effects. URSO, at doses of 15 mg/kg/day used primarily in biliary cirrhosis trials were extremely well tolerated and without toxicity. (Pourpon et al., A multicenter, controlled trial of ursodiol for the treatment of primary biliary cirrhosis. 324 New Engl. J. Med. 1548 (1991)). While the precise mechanism of URSO action is unknown, beneficial effects of URSO therapy are related to the enrichment of the hepatic bile acid pool with this hydrophilic bile acid. It has thus been hypothesized that bile acids more hydrophilic than URSO will have even greater beneficial effects than URSO. For example, tauroursodeoxycholate (TURSO) the taurine conjugate of URSO. Non-steroidal anti-inflammatory drugs (NSAIDs) can inhibit the neoplastic transformation of colorectal epithelium. The likely mechanism to explain this chemopreventive effect is inhibition of prostaglandin synthesis. NSAIDs inhibit cyclooxygenase, the enzyme that converts arachidonic acid to prostaglandins and thromboxanes. However, the potential chemopreventive benefits of NSAIDs such as sulindac or mesalamine are tempered by their well known toxicities and moderately high risk of intolerance. Abdominal pain, dispepsia, nausea, diarrhea, constipation, rash, dizziness, or headaches have been reported in up to 9% of patients. The elderly appear to be particularly vulnerable as the incidence of NSAID-induced gastroduodenal ulcer disease, including gastrointestinal bleeding, is higher in those over the age of 60; this is also the age group most likely to develop colon cancer, and therefore most likely to benefit from chemoprevention. The gastrointestinal side effects associated with NSAID use result from the inhibition of Cox-1, an enzyme responsible for maintenance of the gastric mucosa. Therefore, the use of a Cox-2 selective inhibiting agent and a DNA topoisomerase I inhibiting agent in combination with URSO is contemplated to treat or prevent cancer, including but not limited to colon cancer or colonic polyps; it is contemplated that this treatment will result in lower gastrointestinal side effects than the combination of standard NSAIDs and URSO.

An additional class of antineoplastic agents that may be used in the methods, combinations and compositions of the present invention include nonsteroidal antiinflammatory drugs (NSAIDs). NSAIDs have been found to prevent the production of prostaglandins by inhibiting enzymes in the human arachidonic acid/prostaglandin pathway, including the enzyme cyclooxygenase (Cox). However, for the purposes of the present invention the definition of an NSAID does not include the “selective Cox-2 inhibiting agents” described herein. Thus the phrase “nonsteroidal antiinflammatory drug” or “NSAID” includes agents that specifically inhibit Cox-1, without significant inhibition of Cox-2; or inhibit Cox-1 and Cox-2 at substantially the same potency. The potency and selectivity for the enzyme Cox-1 and Cox-2 can be determined by assays well known in the art, see for example, Cromlish and Kennedy, Biochemical Pharmacology, Vol. 52, pp 1777-1785,1996.

Examples of NSAIDs that can be used in the combinations of the present invention include sulindac, indomethacin, naproxen, diclofenac, tolectin, fenoprofen, phenylbutazone, piroxicam, ibuprofen, ketophen, mefenamic acid, tolmetin, flufenamic acid, nimesulide, niflumic acid, piroxicam, tenoxicam, phenylbutazone, fenclofenac, flurbiprofen, ketoprofen, fenoprofen, acetaminophen, salicylate and aspirin.

Additionally, it has been recently discovered in vitro that Cox-2 expression is upregulated in cells overexpressing the HER-2/neu oncogene. (Subbaramaiah et al., Increased expression of Cox-2 in HER-2/neu-overexpressing breast cancer. Cancer Research (submitted for publication Fall 1999)). In this study, markedly increased levels of PGE₂ production, Cox-2 protein and mRNA were detected in HER-2/neu transformed mammary epithelial cells compared to a non-transformed partner cell line. Amplification and/or overexpression of HER-2/nue (ErbB2) occurs in 20-30% of human breast and ovarian cancers as well as in 5-15% of gastric and esophageal cancers and is associated with poor prognosis. Products of Cox-2 activity, i.e., prostaglandins, stimulate proliferation, increase invasiveness of malignant cells, and enhance the production of vascular endothelial growth factor, which promotes angiogenesis. Further, HER-2/neu induces the production of angiogenic factors such as vascular endothelial growth factor.

Consequently, the administration of an anti HER-2/neu antibodies such as trastuzumab (Herceptin®) and other therapies directed at inhibiting HER-2/neu, in combination with a Cox-2 selective inhibiting agent and a DNA topoisomerase I inhibiting agent is contemplated to prevent or treat cancers in which HER-2/neu is overexpressed.

Methods for the production of anti-ErbB2 antibodies are described in WO 99/31,140.

Molecular Tumor Markers

The term “tumor marker” or “tumor biomarker” encompasses a wide variety of molecules with divergent characteristics that appear in body fluids or tissue in association with a clinical tumor and also includes tumor-associated chromosomal changes. Tumor markers fall primarily into three categories: molecular or cellular markers, chromosomal markers, and serological or serum markers. Molecular and chromosomal markers complement standard parameters used to describe a tumor (i.e. histopathology, grade, tumor size) and are used primarily in refining disease diagnosis and prognosis after clinical manifestation. Serum markers can often be measured many months before clinical tumor detection and are thus useful as an early diagnostic test, in patient monitoring, and in therapy evaluation.

Molecular markers of cancer are products of cancer cells or molecular changes that take place in cells because of activation of cell division or inhibition of apoptosis. Expression of these markers can predict a cell's malignant potential. Because cellular markers are not secreted, tumor tissue samples are generally required for their detection. Non-limiting examples of molecular tumor markers that can be used in the methods, combinations and compositions of the present invention are listed in Table No. 18, below.

TABLE No. 18
Non-limiting Examples of Molecular Tumor Markers

| Tumor | Marker |
|---|---|
| Breast | p53 |
| Breast, Ovarian | ErbB-2/Her-2 |
| Breast | S phase and ploidy |
| Breast | pS2 |
| Breast | MDR2 |
| Breast | urokinase plasminogen activator |
| Breast, Colon, | myc family |
| Lung | |

Chromosomal Tumor Markers

Somatic mutations and chromosomal aberrations have been associated with a variety of tumors. Since the identification of the Philadelphia Chromosome by Nowel and Hungerford, a wide effort to identify tumor-specific chromosomal alterations has ensued. Chromosomal cancer markers, like cellular markers, are can be used in the diagnosis and prognosis of cancer. In addition to the diagnostic and prognostic implications of chromosomal alterations, it is hypothesized that germ-line mutations can be used to predict the likelihood that a particular person will develop a given type of tumor. Non-limiting examples of chromosomal tumor markers that can be used in the methods, combinations and compositions of the present invention are listed in Table No. 19, below.

TABLE No. 19
Non-limiting Examples of Chromosomal Tumor
Markers

| Tumor | Marker |
|---|---|
| Breast | 1p36 loss |
| Breast | 6q24-27 loss |
| Breast | 11q22-23 loss |
| Breast | 11q13 amplification |
| Breast | TP53 mutation |
| Colon | Gain of chromosome 13 |
| Colon | Deletion of short arm of chromosome 1 |
| Lung | Loss of 3p |
| Lung | Loss of 13q |
| Lung | Loss of 17p |
| Lung | Loss of 9p |

Serological Tumor Markers

Serum markers including soluble antigens, enzymes and hormones comprise a third category of tumor markers. Monitoring serum tumor marker concentrations during therapy provides an early indication of tumor recurrence and of therapy efficacy. Serum markers are advantageous for patient surveillance compared to chromosomal and cellular markers because serum samples are more easily obtainable than tissue samples, and because serum assays can be performed serially and more rapidly. Serum tumor markers can be used to determine appropriate therapeutic doses within individual patients. For example, the efficacy of a combination regimen consisting of chemotherapeutic and antiangiogenic agents can be measured by monitoring the relevant serum cancer marker levels. Moreover, an efficacious therapy dose can be achieved by modulating the therapeutic dose so as to keep the particular serum tumor marker concentration stable or within the reference range, which may vary depending upon the indication. The amount of therapy can then be modulated specifically for each patient so as to minimize side effects while still maintaining stable, reference range tumor marker levels. Table No. 20 provides non-limiting examples of serological tumor markers that can be used in the present invention.

TABLE No. 20
Non-limiting Examples of Serum Tumor Markers

| Cancer Type | Marker |
|---|---|
| Germ Cell Tumors | a-fetoprotein (AFP) |
| Germ Cell Tumors | human chorionic gonadotrophin (hCG) |
| Germ Cell Tumors | placental alkaline phosphatase (PLAP) |
| Germ Cell Tumors | lactate dehydrogenase (LDH) |
| Prostate | prostate specific antigen (PSA) |
| Breast | carcinoembryonic antigen (CEA) |
| Breast | MUC-1 antigen (CA15-3) |
| Breast | tissue polypeptide antigen (TPA) |
| Breast | tissue polypeptide specific antigen |
| | (TPS) |
| Breast | CYFRA 21.1 |
| Breast | soluble erb-B-2 |
| Ovarian | CA125 |
| Ovarian | OVX1 |
| Ovarian | cancer antigen CA72-4 |
| Ovarian | TPA |
| Ovarian | TPS |
| Gastrointestinal | CD44v6 |
| Gastrointestinal | CEA |
| Gastrointestinal | cancer antigen CA19-9 |
| Gastrointestinal | NCC-ST-439 antigen (Dukes C) |
| Gastrointestinal | cancer antigen CA242 |
| Gastrointestinal | soluble erb-B-2 |
| Gastrointestinal | cancer antigen CA195 |
| Gastrointestinal | TPA |
| Gastrointestinal | YKL-40 |
| Gastrointestinal | TPS |
| Esophageal | CYFRA 21-1 |
| Esophageal | TPA |
| Esophageal | TPS |
| Esophageal | cancer antigen CA19-9 |
| Gastric Cancer | CEA |
| Gastric Cancer | cancer antigen CA19-9 |
| Gastric Cancer | cancer antigen CA72-4 |
| Lung | neruon specific enolase (NSE) |
| Lung | CEA |
| Lung | CYFRA 21-1 |
| Lung | cancer antigen CA 125 |
| Lung | TPA |
| Lung | squamous cell carcinoma antigen (SCC) |
| Pancreatic cancer | ca19-9 |
| Pancreatic cancer | ca50 |
| Pancreatic cancer | ca119 |
| Pancreatic cancer | ca125 |
| Pancreatic cancer | CEA |
| Renal Cancer | CD44v6 |
| Renal Cancer | E-cadherin |
| Renal Cancer | PCNA (proliferating cell nuclear antigen) |

### EXAMPLES

Germ Cell Cancers

Non-limiting examples of tumor markers useful in the methods, combinations and compositions of the present invention for the detection of germ cell cancers include, but are not limited to, a-fetoprotein (AFP), human chorionic gonadotrophin (hCG) and its beta subunit (hCGb), lactate dehydrogenase (LDH), and placental alkaline phosphatase (PLAP).

AFP has an upper reference limit of approximately −10 kU/L after the first year of life and may be elevated in germ cell tumors, hepatocellular carcinoma and also in gastric, colon, biliary, pancreatic and lung cancers. AFP serum half life is approximately five days after orchidectomy. According to EGTM recommendations, AFP serum levels less than 1,000 kU/L correlate with a good prognosis, AFP levels between 1,000 and 10,000 kU/L, inclusive, correlate with intermediate prognosis, and AFP levels greater than 10,000 U/L correlate with a poor prognosis.

HCG is synthesized in the placenta and is also produced by malignant cells. Serum hCG concentrations may be increased in pancreatic adenocarcinomas, islet cell tumors, tumors of the small and large bowel, hepatoma, stomach, lung, ovaries, breast and kidney. Because some tumors only hCGb, measurement of both hCG and hCGb is recommended. Normally, serum hCG in men and pre-menopausal women is as high as −5 U/L while post-menopausal women have levels up to −10 U/L. Serum half life of hCG ranges from 16-24 hours. According to the EGTM, hCG serum levels under 5000 U/L correlate with a good prognosis, levels between 5000 and 50000 U/L, inclusively correlate with an intermediate prognosis, and hCG serum levels greater than 50000 U/L correlate with a poor prognosis. Further, normal hCG half lives correlate with good prognosis while prolonged half lives correlate with poor prognosis.

LDH is an enzyme expressed in cardiac and skeletal muscle as well as in other organs. The LDH-1 isoenzyme is most commonly found in testicular germ cell tumors but can also occur in a variety of benign conditions such as skeletal muscle disease and myocardial infarction. Total LDH is used to measure independent prognostic value in patients with advanced germ cell tumors. LDH levels less than 1.5× the reference range are associated with a good prognosis, levels between 1.5 and 10× the reference range, inclusive, are associated with an intermediate prognosis, and levels more than 10× the reference range are associated with a poor prognosis.

PLAP is a enzyme of alkaline phosphatase normally expressed by placental syncytiotrophoblasts. Elevated serum concentrations of PLAP are found in seminomas, non-seminomatous tumors, and ovarian tumors, and may also provide a marker for testicular tumors. PLAP has a normal half life after surgical resection of between 0.6 and 2.8 days.

Prostate Cancer

A non-limiting example of a tumor marker useful in the methods, combinations and compositions of the present invention for the detection of prostate cancer is prostate specific antigen (PSA). PSA is a glycoprotein that is almost exclusively produced in the prostate. In human serum, uncomplexed f-PSA and a complex of f-PSA with a1-anthichymotrypsin make up total PSA (t-PSA). T-PSA is useful in determining prognosis in patients that are not currently undergoing anti-androgen treatment. Rising t-PSA levels via serial measurement indicate the presence of residual disease.

In 1993, the molecular cloning of a prostate-specific membrane antigen (PSMA) was reported as a potential prostate carcinoma marker and hypothesized to serve as a target for imaging and cytotoxic treatment modalities for prostate cancer. Antibodies against PSMA have been described and examined clinically for diagnosis and treatment of prostate cancer. In particular, Indium-111 labelled PSMA antibodies have been described and examined for diagnosis of prostate cancer and itrium-labelled PSMA antibodies have been described and examined for the treatment of prostate cancer.

Breast Cancer

Non-limiting examples of serum tumor markers useful in the methods, combinations and compositions of the present invention for the detection of breast cancer include, but is not limited to, carcinoembryonic antigen (CEA) and MUC-1 (CA 15.3). Serum CEA and CA15.3 levels are elevated in patients with node involvement compared to patients without node involvement, and in patients with larger tumors compared to smaller tumors. Normal range cutoff points (upper limit) are 5-10 mg/L for CEA and 35-60 u/ml for CA15.3. Additional specificity (99.3%) is gained by confirming serum levels with two serial increases of more than 15%.

Ovarian Cancer

A non-limiting example of a tumor marker useful in the methods, combinations and compositions of the present invention for the detection of ovarian cancer is CA125. Normally, women have serum CA125 levels between 0-35 kU/L; 99% of post-menopausal women have levels below 20 kU/L. Serum concentration of CA125 after chemotherapy is a strong predictor of outcome as elevated CA125 levels are found in roughly 80% of all patients with epithelial ovarian cancer. Further, prolonged CA125 half-life or a less than 7-fold decrease during early treatment is also a predictor of poor disease prognosis.

Gastrointestinal Cancers

A non-limiting example of a tumor marker useful in the methods, combinations and compositions of the present invention for the detection of colon cancer is carcinoembryonic antigen (CEA). CEA is a glycoprotein produced during embryonal and fetal development and has a high sensitivity for advanced carcinomas including those of the colon, breast, stomach and lung. High pre- or postoperative concentrations (>2.5 ng/ml) of CEA are associated with worse prognosis than are low concentrations. Further, some studies in the literature report that slow rising CEA levels indicates local recurrence while rapidly increasing levels suggests hepatic metastasis.

Lung Cancer

Examples of serum markers useful in the methods, combinations and compositions of the present invention to monitor lung cancer therapy include, but are not limited to, CEA, cytokeratin 19 fragments (CYFRA 21-1), and Neuron Specific Enolase (NSE).

NSE is a glycolytic isoenzyme of enolase produced in central and peripheral neurons and malignant tumors of neuroectodermal origin. At diagnosis, NSE concentrations greater than 25 ng/mL are suggestive of malignancy and lung cancer while concentrations greater than 100 ng/mL are suggestive of small cell lung cancer.

CYFRA 21-1 is a tumor marker test which uses two specific monoclonal antibodies against a cytokeratin 19 fragment. At diagnosis, CYFRA 21-1 concentrations greater than 10 ng/mL are suggestive of malignancy while concentrations greater than 30 ng/mL are suggestive of lung cancer.

Accordingly, dosing of the Cox-2 selective inhibiting agent (or prodrug thereof) and the DNA topoisomerase I inhibiting agents (or other combination therapies of the present invention) may be determined and adjusted based on measurement of tumor markers in body fluids or tissues, particularly based on tumor markers in serum. For example, a decrease in serum marker level relative to baseline serum marker prior to administration of the Cox-2 inhibitor and the DNA topoisomerase I inhibiting agents indicates a decrease in cancer-associated changes and provides a correlation with inhibition of the cancer. In one embodiment, therefore, the method of the present invention comprises administering the Cox-2 selective inhibiting agent and the DNA topoisomerase I inhibiting agents at doses that in combination result in a decrease in one or more tumor markers, particularly a decrease in one or more serum tumor markers, in the mammal relative to baseline tumor marker levels.

Similarly, decreasing tumor marker concentrations or serum half lives after administration of the combination indicates a good prognosis, while tumor marker concentrations which decline slowly and do not reach the normal reference range predict residual tumor and poor prognosis. Further, during follow-up therapy, increases in tumor marker concentration predicts recurrent disease many months before clinical manifestation.

In addition to the above examples, Table No. 21, below, lists several references that describe tumor markers and their use in detecting and monitoring tumor growth and progression.

TABLE No. 21
Tumor marker references.

European Group on Tumor Markers Publications Committee. Consensus
Recommendations. Anticancer Research 19: 2785-2820 (1999)
Human Cytogenetic Cancer Markers. Sandra R. Wolman and Stewart Sell
(eds.). Totowa, New Jersey: Humana Press. 1997
Cellular Markers of Cancer. Carleton Garrett and Stewart Sell (eds.).
Totowa, New Jersey: Human Press. 1995

All of the various cell types of the body can be transformed into benign or malignant neoplasia or tumor cells and are contemplated as objects of the invention. A “benign” tumor cell denotes the non-invasive and non-metastasized state of a neoplasm. In man the most frequent tissue in which neoplasia disease occurs is lung, followed by colorectal, breast, prostate, bladder, pancreas, and then ovary. Other prevalent types of cancer include leukemia, central nervous system cancers, including brain cancer, melanoma, lymphoma, erythroleukemia, uterine cancer, and head and neck cancer.

General Synthetic Procedures for Compounds of Formulas 2 and 3

The compounds of Formulas 2 and 3 can be synthesized according to the following procedures of Schemes 1-16, wherein the R¹-R⁶ substituents are as defined for Formulas I-II, above, except where further noted.

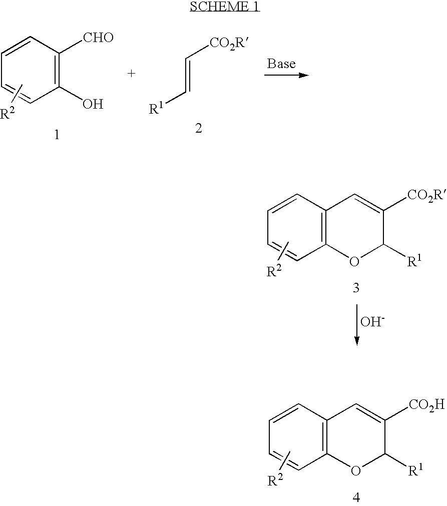

Synthetic Scheme 1 illustrates the general method for the preparation of a wide variety of substituted 2H-1-benzopyran derivatives 3 and 4. In step 1, a representative ortho-hydroxybenzaldehyde (salicylaldehyde) derivative 1 is condensed with an acrylate derivative 2 in the presence of base, such as potassium carbonate in a solvent such as dimethylformamide, to afford the desired 2H-1-benzopyran ester 3. An alternative base-solvent combination for this condensation includes an organic base such as triethylamine and a solvent such as dimethyl sulfoxide. In step 2 the ester is hydrolyzed to the corresponding acid, such as by treatment with aqueous base (sodium hydroxide) in a suitable solvent such as ethanol to afford after acidification the substituted 2H-1-benzopyran-3-carboxylic acid 4.

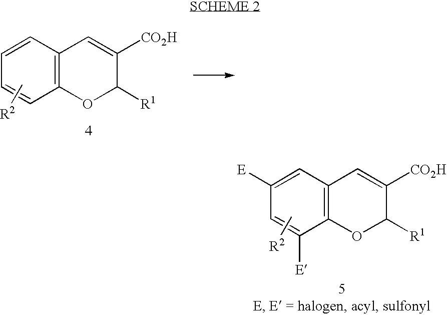

Synthetic Scheme 2 shows the general method for functionalizing selected 2H-1-benzopyrans. Treatment of the 2H-1-benzopyran carboxylic acid 4 or ester 3 with an electrophillic agent makes a 6-substituted 2H-1-benzopyran 5. A wide variety of electrophillic agents react selectively with 2H-1-benzopyrans 4 in the 6-position to provide new analogs in high yield. Electrophillic reagents such as halogen (chlorine or bromine) give the 6-halo derivatives. Chlorosulfonic acid reacts to afford the 6-position sulfonyl chloride that can further be converted to a sulfonamide or sulfone. Friedel-Crafts acylation of 4 provides 6-acylated 2H-1-benzopyrans in good to excellent yield. A number of other electrophiles can be used to selectively react with these 2H-1-benzopyrans in a similar manner. A 6-position substituted 2H-1-benzopyran can react with an electrophilic reagent at the 8-position using similar chemistries to that described for electrophilic substitution of the 6-position. This yields a 2H-1-benzopyran which is substituted at both the 6 and 8 positions.

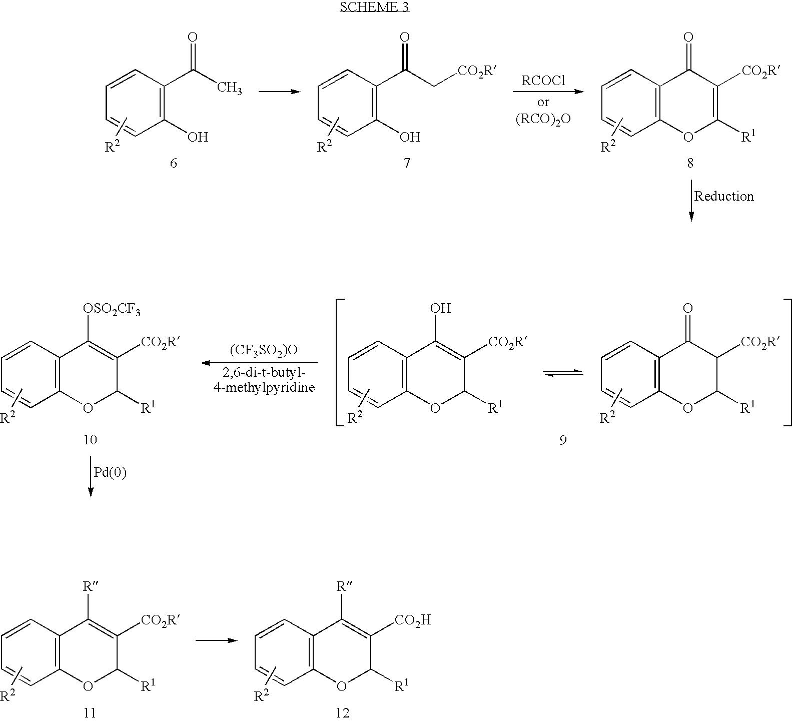

Synthetic Scheme 3 illustrates a second general synthesis of substituted 2H-1-benzopyran-3-carboxylic acids which allows substitution at position 4 of the 2H-1-benzopyran. In this case a commercially or synthetically available subtituted ortho-hydroxy acetophenone 6 is treated with two or more equivalents of a strong base such as lithium bis(trimethylsilyl)amide in a solvent such as tetrahydrofuran (THF), followed by reaction with diethyl carbonate to afford the beta-keto ester 7. Ester 7 is condensed with an acid chloride or anhydride in the presence of a base such as potassium carbonate in a solvent such as toluene with heat to afford 4-oxo-4H-1-benzopyran 8. Reduction of the olefin can be accomplished by a variety of agents including sodium borohydride (NaBH₄) in solvent mixtures such as ethanol and tetrahydrofuran (THF), or by use of triethylsilane in a solvent such as trifluoroacetic acid, or by catalytic reduction using palladium on charcoal and hydrogen gas in a solvent such as ethanol to yield the new beta-keto ester 9 (two tautomeric structures shown). Acylation of the oxygen of the ketone enolate in the presence of a base such as 2,6-di-tert-butyl-4-methylpyridine, an acylating agent such as trifluoromethanesulfonic anhydride, and using a solvent such as methylene chloride yields the enol-triflate 10. Triflate 10 can be reduced with reagents such as tri-n-butyltin hydride, lithium chloride and a palladium (0) catalyst such as tetrakis(triphenylphosphine)palladium (0) in a solvent such as tetrahydrofuran to yield 2H-1-benzopyran ester 11 where R″ is hydrogen. The ester 11 can be saponified with a base such as 2.5 N sodium hydroxide in a mixed solvent such as tetrahydrofuran-ethanol-water (7:2:1) to yield the desired substituted 2H-1-benzopyran-3-carboxylic acid.

To incorporate a carbon fragment R³ one can treat triflate 10 with reagents known to undergo “cross-coupling” chemistries such a tributylethyenyltin, lithium chloride and a palladium(0) catalyst such as tetrakis(triphenylphosphine)palladium (0) in a solvent such as tetrahydrofuran to yield 2H-1-benzopyran ester 11 where R³ is a vinyl moiety. The ester 6 can be saponified with a base such as 2.5 N sodium hydroxide in a mixed solvent such as tetrahydrofuran-ethanol-water (7:2:1) to yield the desired 4-vinyl-2H-1-benzopyran-3-carboxylic acid (12, R″=CH₂CH—). Similarly triflate 10 can be converted under similar conditions using tri-n-butylphenyltin to 2H-1-benzopyran where R³=phenyl and by hydrolysis of the ester converted to the carboxylic acid 12 where R³=phenyl. Using a similar strategy, substituents which be incorporated as substitutent R³ can be substituted olefins, substituted aromatics, substuted heteroaryl, acetylenes and substituted acetylenes.

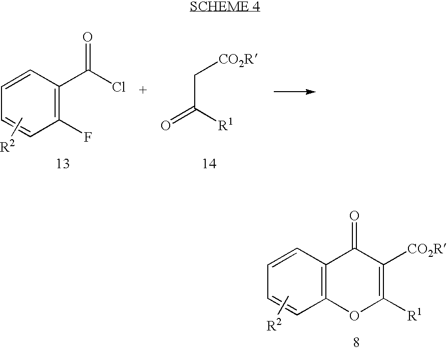

Synthetic Scheme 4 shows an alternative general procedure for the preparation of 4-oxo-4H-1-benzopyran 8. Treatment of an ortho-fluorobenzoyl chloride with an appropriately substituted beta-keto ester 14 with a base such as potassium carbonate in a solvent such as toluene provides 4-oxo-4H-1-benzopyran 8. 4-Oxo-4H-1-benzopyran 8 can be converted to 2H-1-benzopyran 12 as described in Scheme 3.

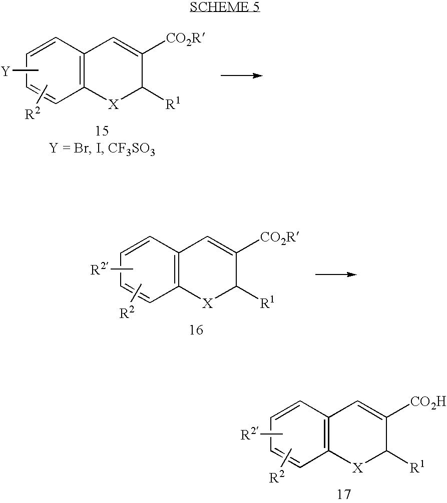

Synthetic Scheme 5 shows a general method for substitution of the aromatic ring of the 2H-1-benzopyran. This can be accomplished through organo-palladium mediated “cross-coupling” chemistries using a palladium (0) catalyst to couple benzopyran 15 at position Y, where Y is iodide, bromide or triflate, with an acetylene, olefin, nitrile, or aryl coupling agent. Substituted acetylenes as the coupling agent will provide the corresponding substituted acetylene. Substituted aryl moieties can be incorporated using arylboronic acids or esters; nitriles can be incorporated by use of zinc (II) cyanide. The resulting ester 16 can be converted to carboxylic acid 17 as described in Scheme 1.

Another approach to substitution of the aryl moiety of the benzopyran 15 is to convert Y, where Y is iodide or bromide, to a perfluoroalkyl moiety. Exemplary of this transformation is the conversion of 15 (Y=iodide) to 16 (R^{2′}=pentafluoroethyl) using a potassium pentafluoropropionate and copper (I) iodide in hexamethylphosphoramide (HMPA). The resulting ester 16 can be converted to carboxylic acid 15 as described in Scheme 1.

A similar method adds substitution of the aromatic ring in dihydroquinoline-3-carboxylates. This can be accomplished through organopalladium couplings with aryl iodides, bromides, or triflates and various coupling agents (R. F. Heck, Palladium Reagents in Organic Synthesis. Academic Press 1985). When using a suitable palladium catalyst such as tetrakis(triphenyl-phospine)palladium(0) in this reaction, coupling agents such as alkynes provide disubstituted alkynes, phenyl boronic acids afford biphenyl compounds, and cyanides produce arylcyano compounds. A number of other palladium catalysts and coupling reagents could be used to selectively react with appropriately substituted dihydroquinoline-3-carboxylates in a similar manner.

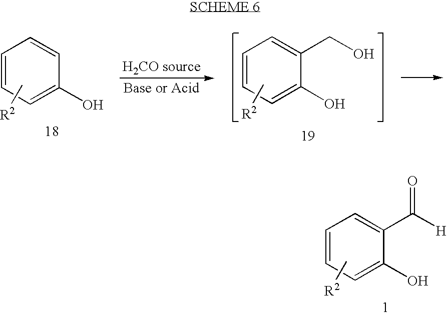

Synthetic Scheme 6 shows a general synthetic route for conversion of a commercially or synthetically available substituted phenol into a substituted salicylaldehyde. Several different methods which utilize formaldehyde or a chemically equivalent reagent are described in detail below.

Reaction of an appropriately substituted phenol 18 in basic media with formaldehyde (or chemical equivalent) will yield the corresponding salicylaldehyde 1. The intermediate, ortho-hydroxymethylphenol 19, will under appropriate reaction conditions be oxidized to the salicylaldehyde 1 in situ. The reaction commonly employs ethyl magnesium bromide or magnesium methoxide(one equivalent) as the base, toluene as the solvent, paraformaldehyde (two or more equivalents) as the source of formaldehyde, and employs hexamethylphoramide (HMPA) or N,N,N′,N′-tetramethylethylenediamine (TMEDA). (See: Casiraghi, G. et al., J. C. S. Perkin I, 1978, 318-321.)

Alternatively an appropriately substituted phenol 18 may react with formaldehyde under aqueous basic conditions to form the substituted ortho-hydroxybenzyl alcohol 19 (See: a) J. Leroy and C. Wakselman, J. Fluorine Chem., 40, 23-32 (1988). b) A. A. Moshfegh, et al., Helv. Chim. Acta., 65, 1229-1232 (1982)). Commonly used bases include aqueous potassium hydroxide or sodium hydroxide. Formalin (38% formaldehyde in water) is commonly employed as the source of formaldehyde. The resulting ortho-hydroxybenzyl alcohol 19 can be converted to the salicylaldehyde 1 by an oxidizing agent such as manganese (IV) dioxide in a solvent such as methylene chloride or chloroform (See: R-G. Xie, et al., Synthetic Commun. 24, 53-58 (1994)).

An appropriately substituted phenol 18 can be treated under acidic conditions with hexamethylenetetramine (HMTA) to prepare the salicylaldehyde 1 (Duff Reaction; See: Y. Suzuki, and H. Takahashi, Chem. Pharm. Bull., 31, 1751-1753 (1983)). This reaction commonly employs acids such as acetic acid, boric acid, methanesulfonic acid, or trifluoromethanesulfonic acid. The source of formaldehyde commonly used is hexamethylenetetramine.

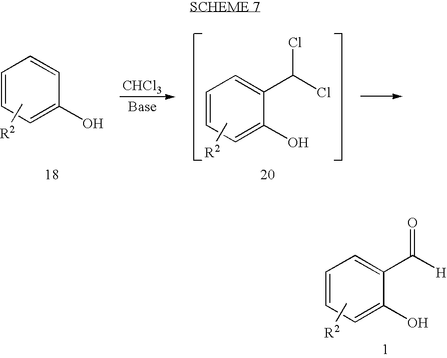

Synthetic Scheme 7 shows the Reimer-Tiemann reaction in which an commercially or synthetically available appropriately substituted phenol 18 will under basic conditions react with chloroform to yield a substituted salicylaldehyde 1 (See: Cragoe, E. J.; Schultz, E. M., U.S. Pat. No. 3,794,734,1974).

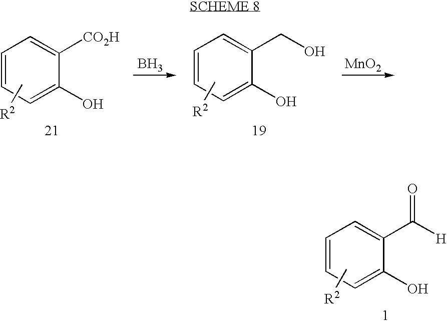

Synthetic Scheme 8 shows the conversion of a commercially or synthetically available appropriately substituted salicylic acid 21 to its respective salicylaldehyde 1 via an intermediate 2-hydroxybenzyl alcohol 19. Reduction of the salicylic acid 21 can be accomplished with a hydride reducing agent such as borane in a solvent such as tetrahydrofuran. Treatment of the intermediate 2-hydroxybenzyl alcohol 19 with an oxidizing agent such as manganese (IV) oxide in a solvent such as methylene chloride or chloroform provides salicylaldehyde 1.

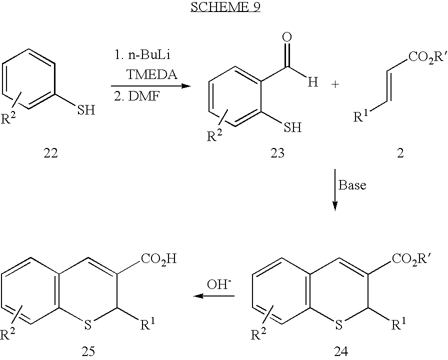

Synthetic Scheme 9 illustrates a general synthetic method for preparation of a wide variety of substituted 2-(trifluoromethyl)-2H-1-benzothiopyran-3-carboxylic acids (25). In step 1, an appropriately commercially or synthetically available substituted thiophenol 22 is ortho-metallated with a base such as n-butyllithium employing TMEDA (N,N,N′,N′-tetramethylethylenediamine) followed by treatment with dimethylformamide to provide the 2-mercaptobenzaldehyde 23. Condensation of the 2-mercaptobenzaldehyde 23 with an acrylate 2 in the presence of base provides ester 24 which can be saponified in the presence of aqueous base to afford the substituted 2H-1-benzothiopyran-3-carboxylic acids 25.

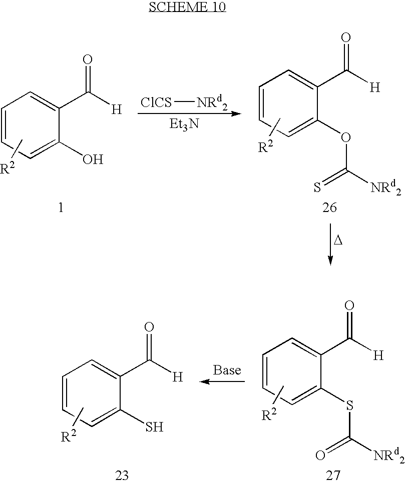

Synthetic Scheme 10 shows a method for preparing a substituted 2-mercaptobenzaldehyde from an appropriate commercially or synthetically available substituted salicylaldehyde. In step 1, the phenolic hydroxyl of salicylaldehyde 1 is converted to the corresponding O-aryl thiocarbamate 26 by acylation with an appropriately substituted thiocarbamoyl chloride such as N,N-dimethylthiocarbamoyl chloride in a solvent such as dimethylformamide using a base such as triethylamine. In Step 2, O-aryl thiocarbamate 26 rearranges to S-aryl thiocarbamate 27 when heated sufficiently such as to 200° C. using either no solvent or a solvent such as N,N-dimethylaniline (See: A. Levai, and P. Sebok, Synth. Commun., 221735-1750 (1992)). Hydrolysis of S-aryl thiocarbamate 27 with a base such as 2.5 N sodium hydroxide in a solvent mixture such as tetrahydrofuran and ethanol yields the substituted 2-mercaptobenzaldehyde 23 which can be converted to the substituted 2H-1-benzothiopyran-3-carboxylic acids 25 as described in Scheme 9.

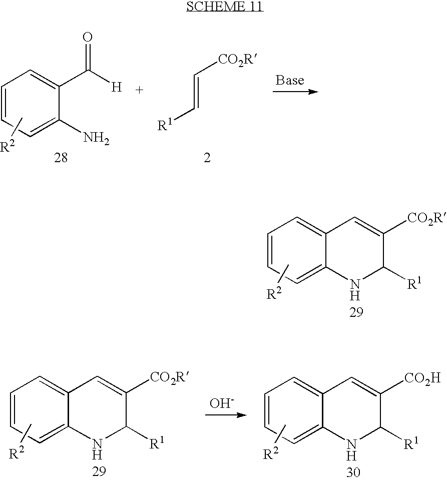

Synthetic Scheme 11 illustrates the general method for the preparation of a wide variety of dihydroquinoline-3-carboxylic acid derivatives 30. R² represents the aromatic substitution of commercially and synthetically available 2-aminobenzaldeydes 28. The 2-amino-benzaldehyde derivative 28, where R² represents various substitutions, is condensed with a acrylate derivative 2 in the presence of base such as potassium carbonate, triethylamine, or diazbicyclo[2.2.2]undec-7-ene in solvents such as dimethylformamide to afford the dihydroquinoline-3-carboxylate esters 29. The ester 29 can be saponified to the corresponding acid, such as by treatment with aqueous inorganic base such as 2.5 N sodium hydroxide in a suitable solvent such as ethanol to afford after acidification the desired dihydroquinoline-3-carboxylic acid 30.

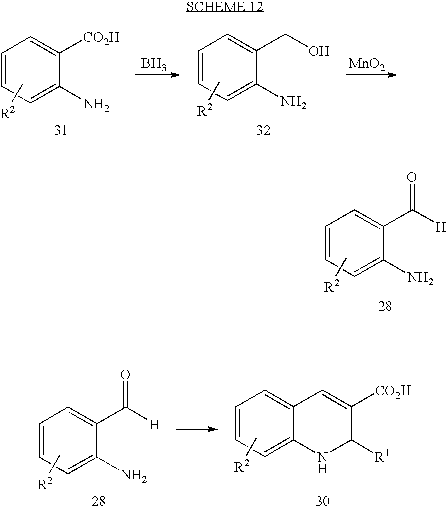

Synthetic Scheme 12 illustrates the preparation of dihydroquinoline-3-carboxylic acid 30 from 2-aminobenzoic acids 31. R² represents the aromatic substitution of commercially and synthetically available 2-aminobenzoic acids 31. Reduction of the representative 2-aminobenzoic acid 31 to the desired 2-aminobenzyl alcohol 32 was accomplished with a hydride reducing agent such as borane in a solvent such as tetrahydrofuran. Treatment of the desired 2-aminobenzyl alcohol 32 with an oxidizing agent such as manganese(IV)oxide in a solvent such as methylene chloride provides the representative 2-aminobenzaldehydes 28. (C. T. Alabaster, et al. J. Med. Chem. 31, 2048-2056 (1988)) The 2-aminobenzaldehydes were converted to the desired dihydroquinoline-3-carboxylic acid 30 as described in Scheme 11.

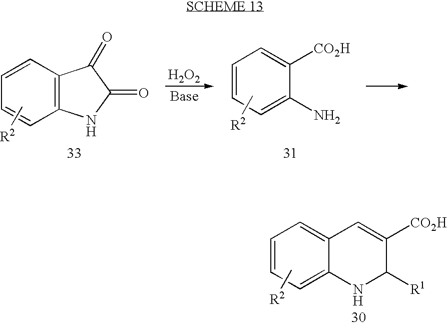

Synthetic Scheme 13 illustrates the general method for the preparation of a wide variety of dihydroquinoline-3-carboxylic acid derivatives 30 from isatins 33. R² represents the aromatic substitution of commercially and synthetically available isatins 33. A representative isatin 33 was treated with basic peroxide generated from hydrogen peroxide and a base such as sodium hydroxide to afford the desired representative 2-aminobenzoic acids 31. (M. S. Newman and M. W. Lougue, J. Org. Chem., 36, 1398-1401 (1971)) The 2-aminobenzoic acids 31 are subsequently converted to the desired dihydroquinoline-3-carboxylic acid derivatives 30 as described in synthetic Scheme 12.

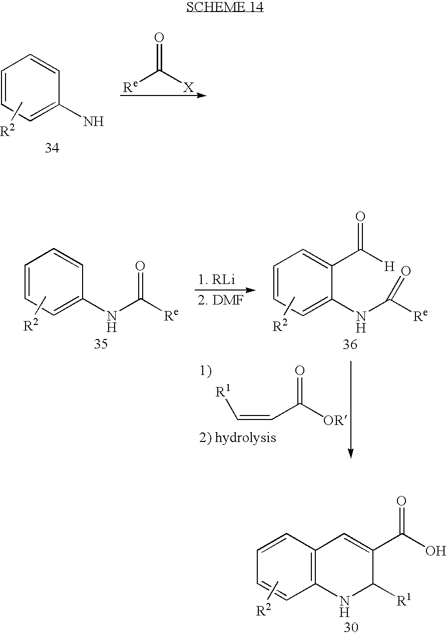

Synthetic Scheme 14 is another general method for the preparation of dihydroquinoline-3-carboxylic acid derivatives 30. In step 1, an appropriate commercially or synthetically available substituted aniline 34 can be treated with an acylating reagent such as pivaloyl chloride yielding an amide 35. The ortho-dianion of amide 35 is prepared by treating amide 35 with organo-lithium bases such as n-butyllithium or tert-butyllithium in tetrahydrofuran at low temperature. The dianion is quenched with dimethylformamide to afford the acylated-2-amino-benzaldehydes 36. (J. Turner, J. Org. Chem., 48, 3401-3408 (1983)) Reaction of these aldehydes in the presence of bases such as lithium hydride with a acrylate followed by work up with aqueous inorganic bases and hydrolysis, such as by treatment with aqueous base (sodium hydroxide) in a suitable solvent such as ethanol affords, after acidification, a dihydroquinoline-3-carboxylic acid 30.

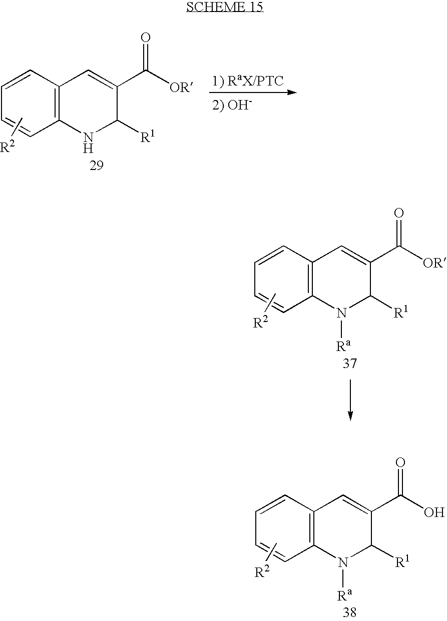

Synthetic Scheme 15 shows a general method for alkylation of the nitrogen of dihydroquinoline-3-carboxylate ester derivatives 29. The step involves treatment of dihydroquinoline-3-carboxylate ester derivatives 29 with alkyl halides such as iodoethane in the presence of phase transfer catalysts such a tetrabutylammonium iodide, and a base such as caustic (50% aqueous sodium hydroxide) in a solvent such as dichloromethane. These conditions afford the N-alkylated dihyrdoquinoline-3-carboxylate esters 37. Saponification of 37 with aqueous base provides N-alkylated-dihyroquinoline-3-carboxylic acid derivatives 38.

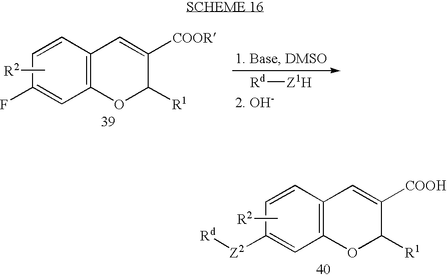

Synthetic Scheme 16 shows a general method for the preparation of a 7-ether (Z¹=O) or thioether (Z¹=S) substituted benzopyran-3-carboxylic ester. An appropriately substituted phenol, thiophenol, hydroxy-heterocycle, mercaptoheterocycle, alcohol, or alkylthiol can be condensed under basic conditions using a base such as potassium carbonate in a solvent such as dimethysulfoxide, at temperature above room temperature, such as 100° C., with an appropriately substituted 7-fluorobenzopyran derivative 30 to yield the corresponding ether or thioether. Hydrolysis of the ester with an aqueous base such as lithium hydroxide or sodium hydroxide in a solvent mixture such as tetrahydrofuran-ethanol-water yields acid 40. When appropriate, a thioether (Z²=S) can be oxidized to the sulfoxide (Z²=SO) or sulfone (Z²=SO₂) with an oxidant such as OXONE® or m-CPBA either before or after ester hydrolysis. In this chemistry Rd can include aryl, heteroaryl, heterocyclic, alicyclic, branched or linear aliphatic, branched or linear perfluoro-aliphatic moiety.

The following examples contain detailed descriptions of the methods of preparation of compounds of Formulas 2 and 3. These detailed descriptions fall within the scope, and serve to exemplify, the above described General Synthetic Procedures which form part of the invention. These detailed descriptions are presented for illustrative purposes only and are not intended as a restriction on the scope of the invention. All parts are by weight and temperatures are in degrees centigrade unless otherwise indicated. All compounds showed NMR spectra consistent with their assigned structures.

The following abbreviations are used:

HCl—hydrochloric acid

MgSO4—magnesium sulfate

Na2SO4—sodium sulfate

DMF—dimethylformamide

THF—tetrahydrofuran

NaOH—sodium hydroxide

EtOH—ethanol

K2CO3—potassium carbonate

CDCl3—deuterated chloroform

CD3OD—deuterated methanol

Et2O—diethyl ether

EtOAc—ethyl acetate

NaHCO3— sodium bicarbonate

KHSO4— potassium sulfate

NaBH4—sodium borohydride

### Example 1

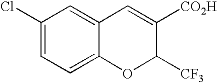

### 6-Chloro-2-trifluoromethyl-2H-1-benzopyran-3-carboxylic Acid

Step 1. Preparation of ethyl 6-chloro-2-trifluoromethyl-2H-1-benzopyran-3-carboxylate.

A mixture of 5-chlorosalicylaldehyde (20.02 g, 0.128 mole) and ethyl 4,4,4-trifluorocrotonate (23.68 g, 0.14 mole) was dissolved in anhydrous DMF, warmed to 60° C. and treated with anhydrous K₂CO₃ (17.75 g, 0.128 mole). The solution was maintained at 60° C. for 20 hours, cooled to room temperature, and diluted with water. The solution was extracted with ethyl acetate. The combined extracts were washed with brine, dried over anhydrous MgSO₄, filtered and concentrated in vacuo to afford 54.32 g of an oil. The oil was dissolved in 250 mL of methanol and 100 mL of water, whereupon a white solid formed that was isolated by filtration, washed with water and dried in vacuo, to afford the ester as a yellow solid (24.31 g, 62%): mp 62-64° C. ¹H NMR (CDCl3/90 MHz) 7.64 (s, 1H), 7.30-7.21 (m, 2H), 6.96 (d, 1H, J=Hz), 5.70 (q, 1H, J=Hz), 4.30 (q, 2H, J=7.2 Hz), 1.35 (t, 3H, J=7.2 Hz).

Step 2. Preparation of 6-chloro-2-trifluoromethyl-2H-1-benzopyran-3-carboxylic Acid.

A solution of the ester from Step 1 (13.02 g, 42 mmole) was dissolved in 200 mL of methanol and 20 mL of water, treated with lithium hydroxide (5.36 g, 0.128 mole) and stirred at room temperature for 16 hours. The reaction mixture was acidified with 1.2 N HCl, whereupon a solid formed that was isolated by filtration. The solid was washed with 200 mL of water and 200 mL of hexanes and dried in vacuo to afford the title compound as a yellow solid (10.00 g, 85%): mp 181-184° C.

### Example 2

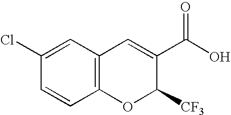

### (S)-6-Chloro-2-trifluoromethyl-2H-1-benzopyran-3-carboxylic Acid

To a solution of 6-chloro-2-trifluoromethyl-2H-1-benzopyran-3-carboxylic acid (Example 1, Step 2)(12.00 g, 43.07 mmol) and (S)(−)-α-methylbenzylamine (2.61 g, 21.54 mmol) in methyl-tert-butyl ether (30 mL) was slowly added n-heptane (200 mL) until the mixture became cloudy. The mixture was heated (steam bath) to boiling and set aside for 24 h during which time crystals formed. Filtration of the suspension yielded a crystalline product (5.5 g) which was recrystallized from methyl-tert-butyl ether (30 mL) and n-heptane (200 mL) yielding upon filtration a white solid (3.1 g). This solid was dissolved in EtOAc (100 mL) and washed with 1 N hydrochloric acid (50 mL) and brine (2×50 mL), dried over MgSO₄ and concentrated in vacuo yielding a white solid. Recrystallization of this solid from methyl-t-butyl ether/n-heptane yielded the title compound as the highly enriched isomer, a white solid (2.7 g, 45%): mp 126.7-128.9° C. ¹H NMR (CDCl3/300 MHz) 7.78 (s, 1H), 7.3-7.1 (m, 3H), 6.94 (d, 1H, J=8.7 Hz), 5.66 (q, 1H, J=6.9 Hz). Anal. Calc'd for C₁₁H₆O₃F₃Cl: C, 47.42; H, 2.17; N, 0.0. Found: C, 47.53; H, 2.14; N, 0.0. This compound was determined to have an optical purity of greater than 90% ee.

Procedure for Determining Optical Purity

To a solution of the free acid (title compound) (0.005 g, 0.017 mmol) in ethyl acetate (1.5 mL) in a test tube was added (trimethylsilyl)diazomethane (30 μL of 2.0 N solution in hexanes, 60 mmol). The resulting yellow solution was warmed until the solution began to gently boil and then was allowed to cool to room temperature and stand for 0.08 hours. With vigorous mixing, the solution was quenched with aqueous 1 N HCl (1.5 mL). The layers were separated and a sample of the ethyl acetate fraction (0.3 mL) was transferred to a vial, concentrated under a stream of nitrogen, was diluted with hexane (total of 1 mL) and a sample (10 μL) analyzed by chiral chromatography. The HPLC utilized a Daicel ChiralPak AD column eluting with 10% isopropanol-hexane at 0.5 mL/min using a UV detector set at 254 nM.

### Example 2

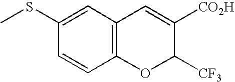

### 6-(Methylthio)-2-(trifluoromethyl)-2H-1-benzopyran-3-carboxylic acid

Step 1. Preparation of 5-(methylthio)salicylaldehyde.

Ethyl magnesium bromide (38 mL of a 3.0 M solution in diethyl ether, 113.8 mmole) was chilled with an ice-water bath. To the chilled solution was added a solution of 4-(methylthio)phenol (15.95 g, 113.8 mmole) in diethyl ether (30 mL) over 0.15 hour during which time gas was evolved. The reaction was held at 0° C. for 0.5 hour, at room temperature for 0.5 hour, and the addition funnel replaced with a distillation head. Toluene (100 mL) was added and the diethyl ether was distilled out of the reactor. The reaction was cooled, toluene (250 mL) and hexamethylphosphoramide (HMPA) (19.8 mL, 20.4 g, 113.8 mmole) were added, and the resulting mixture was stirred for 0.25 hours. The distillation head was replaced with a condenser and paraformaldehyde (8.5 g, 284.4 mmole) was added. The reaction was heated to 90° C. for 3 hours. The reaction mixture was cooled to room temperature, was acidified with 1 N HCl and the layers separated. The organic phase was washed with water, and with brine, dried over MgSO₄, filtered, and concentrated in vacuo to yield a solid. This solid was purified by silica chromatography (hexanes-ethyl acetate, 5:1) yielding the salicylaldehyde as a yellow crystalline solid (6.01 g) of suitable purity to be used in the next reaction without further purification.

Step 2. Preparation of ethyl 6-(methylthio)-2-(trifluoromethyl)-2H-1-benzopyran-3-carboxylate.

5-Methylthiosalicylaldehyde (Step 1)(2.516 g, 14.96 mmole) was added to dimethylformamide (3.5 mL), potassium carbonate (2.27 g, 16.45 mmole) and ethyl 4,4,4-trifluorocrotonate (3.3 mL, 3.8 g, 22.4 mmole). The mixture was heated to 65° C. for 3 h. The reaction was cooled to room temperature, poured into H₂O (50 mL), and extracted with diethyl ether (2×75 mL). The combined ethereal phases were washed with aqueous NaHCO₃ solution (3×50 mL), aqueous 2 N HCl solution (3×50 mL), and brine (3×50 mL), dried over MgSO₄, filtered, diluted with isooctane and partially concentrated in vacuo causing the precipitation of the ethyl ester (2.863 g, 60%) as a yellow powder: mp 87.8-89.6° C. This ester was of suitable purity to use without further purification.

Step 3. Preparation of 6-(methylthio)-2-(trifluoromethyl)-2H-1-benzopyran-3-carboxylic Acid.

The ester (Step 2) was hydrolyzed to form the carboxylic acid via a method similar to that described in Example 1, Step 2: mp 166.3-167.9° C. ¹H NMR (acetone-d₆/300 MHz) 7.87 (s, 1H), 7.43 (d, 1H, J=2.2 Hz), 7.33 (dd, 1H, J=8.5, 2.4 Hz), 6.98 (d, 1H, J=8.5 Hz), 5.79 (q, 1H, J=7.0 Hz), 2.48 (s, 3H). FABLRMS m/z 291 (M+H). ESHRMS m/z 289.0152 (M−H, Calc'd 289.0146). Anal. Calc'd for C₁₂H₉F₃O₃S₁: C, 49.66; H, 3.13; S, 11.05. Found: C, 49.57; H,3.02; S, 11.37.

### Example 3

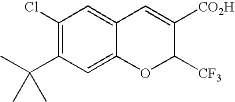

### 6-Chloro-7-(1,1-dimethylethyl)-2-trifluoromethyl-2H-1-benzopyran-3-carboxylic Acid

Step 1. Preparation of 4-tert-butylsalicylaldehyde.

A five liter three-neck round bottom flask equipped with overhead mechanical stirrer and condenser was charged with trifluoroacetic acid (2.4 L). A mixture of 3-tert-butylphenol (412 g, 2.8 mole) and HMTA (424 g, 3.0 mole) was added portion-wise causing an exotherm. With cooling, the temperature was maintained under 80° C. The reaction was heated at 80° C. for one hour, then cooled, and water (2 L) added. After 0.5 hour additional water (4 L) was added and the mixture was extracted with ethyl acetate (6 L). The organic extract was washed with water and brine. The resulting organic phase was divided into 2 L volumes and each diluted with water (1 L), and solid NaHCO₃ added until the mixture was neutralized. The organic phases were isolated and combined, dried over MgSO₄, filtered and concentrated in vacuo yielding an oil. This oil was distilled at 95° C. (0.8 mm) yielding the desired salicylaldehyde as an oil (272.9 g, 56%) which was of sufficient purity to be used without further purification.

Step 2. Preparation of ethyl 7-(1,1-dimethylethyl)-2-(trifluoromethyl)-2H-1-benzopyran-3-carboxylate.

A one liter three-neck flask was charged with 4-tert-butylsalicylaldehyde (Step 1)(100.0 g, 0.56 mole), dimethylformamide (110 mL), and potassium carbonate (79.9 g, 0.58 mole) causing the temperature of the mixture to rise to 40° C. Ethyl 4,4,4-trifluorocrotonate (118.0 g, 0.70 mole) in dimethylformamide (110 mL) was added and the mixture heated to 60° C. at which time the reaction temperature rose to 70° C. The reaction was cooled to 60° C., maintained at 60° C. (with added heating) for 8.5 hours and cooled to room temperature. Ethyl acetate (600 mL) and 3 N HCl (600 mL) were added, mixed, and the layers separated. The aqueous phase was extracted with ethyl acetate and the organic phases were combined. The combined organic phases were washed with brine-water (1:1), brine, dried over MgSO₄, filtered and concentrated in vacuo, yielding a semi-solid. Hexane (600 mL) was added with mixing and the mixture was filtered. The filtrate was washed with brine, dried over MgSO₄, filtered and concentrated in vacuo yielding a solid. This solid was dissolved in hot ethanol (600 mL). Water (190 mL) was added which induced crystallization. Filtration of the mixture and drying of the product provided the desired ester as a crystalline solid (131.3 g, 71%): mp 91.0-94.9° C. This material was of suitable purity to be used in subsequent steps without further purification.

Step 3. Preparation of Ethyl 6-chloro-7-(1,1-dimethylethyl)-2-(trifluoromethyl)-2H-1-benzopyran-3-carboxylate.

A one liter three-neck flask equipped with mechanical stirrer and gas inlet tube was charged with the ester (Step 2) (100 g, 0.3 mole) and acetic acid (300 mL). While cooling (water bath) the reaction mixture, chlorine gas (37.6 g, 0.53 mole) was added which caused the temperature to rise to 48° C. After stirring for two hours, the reaction was cooled in an ice-water bath to 15° C. Zinc powder (19.5 g, 0.3 mole) was added in one portion which caused the temperature to rise to 72° C. After cooling to room temperature additional zinc powder (5.0 g, 0.08 mole) was added and the mixture was stirred for 0.5 hour longer. The crude mixture was filtered through diatomaceous earth and was concentrated in vacuo yielding an oil. The oil was dissolved in ethyl acetate (700 mL) washed with brine-water (1:1, 1 L) and brine (0.5 L). The resulting aqueous phase was extracted with ethyl acetate (700 mL). This ethyl acetate phase was washed with brine-water (1:1, 1 L) and brine (0.5 L). The combined organic phases were dried over MgSO₄, filtered and concentrated in vacuo yielding the title compound as a yellow oil (116 g, 106%). This material, which contained some entrained ethyl acetate, was of suitable purity to be used in subsequent steps without further purification.

Step 4. Preparation of 6-chloro-7-(1,1-dimethylethyl)-2-(trifluoromethyl)-2H-1-benzopyran-3-carboxylic Acid.

To a solution of the ester (Step 3) (116 g, 0.3 mole) in methanol (500 mL) and tetrahydrofuran (500 mL) in a one liter flask was added aqueous sodium hydroxide (2.5 N, 240 mL, 0.6 mole). After stirring overnight, the pH of the solution was adjusted to 1 with concentrated hydrochloric acid and the solution was extracted with ethyl acetate. The ethyl acetate phase was dried over MgSO₄, filtered and concentrated in vacuo yielding a solid. This solid was dissolved in hot ethanol (500 mL). Water (500 mL) was added and upon cooling to room temperature crystals formed which were collected by vacuum filtration. The crystals were washed with ethanol-water (3:7, 3×200 mL) and dried providing the title acid as a crystalline solid (91.6 g, 91%): mp 194.9-196.5° C. ¹H NMR (acetone-d6/300 MHz) 7.86 (s, 1H), 7.52 (s, 1H), 7.12 (s, 1H), 5.83 (q, 1H, J=7.1 Hz), 1.48 (s, 9H). Anal. Calc'd for C15H14ClF3O3: C, 53.83; H, 4.22; Cl, 10.59. Found: C, 53.92; H, 4.24; Cl, 10.50.

### Example 4

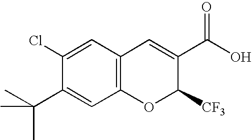

### (S)-6-Chloro-7-(1,1-dimethylethyl)-2-(trifluoromethyl)-2H-1-benzopyran-3-carboxylic Acid

To a solution of 6-chloro-7-(1,1-dimethylethyl)-2-trifluoromethyl-2H-1-benzopyran-3-carboxylic acid (Example 3)(11.4 g, 34.1 mmol) and (S)(−)-2-amino-3-phenyl-1-propanol (2.57 g, 17.00 mmol) was added n-heptane (200 mL) and the mixture set aside for 16 hours. The resulting suspension was filtered yielding a solid (3.8 g). This solid was recrystallized from 2-butanone (20 mL) and n-heptane (200 mL) yielding upon filtration a white solid (3.0 g). This solid was dissolved in ethyl acetate (100 mL) and washed with 1 N HCl (50 mL) and brine (2×50 mL), dried over MgSO₄ and concentrated in vacuo yielding a white solid. This solid was recrystallized from n-heptane yielding the title compound of high optical purity as a crystalline solid (1.7 g, 30%): mp 175.4-176.9° C. ¹H NMR (acetone-d6/300 MHz) 7.86 (s, 1H), 7.52 (s, 1H), 7.12 (s, 1H), 5.83 (q, 1H, J=7.1 Hz), 1.48 (s, 9H). Anal. Calc'd for C₁₅H₁₄O₃F₃Cl: C, 53.83; H, 4.22; N, 0.0; Cl, 10.59. Found: C, 53.78; H, 4.20; N, 0.0; Cl, 10.65. This compound was determined to have an optical purity of greater than 90% ee. Chiral purity was determined as describe in Example 2.

### Example 5

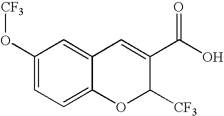

### 6-Trifluoromethoxy-2-trifluoromethyl-2H-1-benzopyran-3-carboxylic Acid

5-(Trifluoromethoxy)salicylaldehyde was converted to the title compound by a similar procedure to that described in Example 1: mp 118.4-119.5° C. ¹H NMR (acetone-d6/300 MHz) 7.95 (s, 1H), 7.54 (d, 1H, J=2.1 Hz), 7.39 (dd, 1H, J=2.4 Hz, and J=9.0 Hz), 7.02 (d, 1H, J=9.0 Hz), 5.88 (q H−F, 1H, J=7.2 Hz). FABHRMS m/z 329.0228 (M+H, Calc'd 329.0249). Anal. Calc'd for C12H6F6O4: C, 43.92; H, 1.84. Found: C, 43.84; H, 1.87.

### Example 6

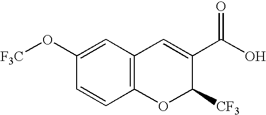

### (S)-6-Trifluoromethoxy-2-trifluoromethyl-2H-1-benzopyran-3-carboxylic Acid

To a solution of 6-trifluoromethoxy-2-(trifluoromethyl)-2H-1-benzopyran-3-carboxylic acid (Example 5)(17.72 g, 54.00 mmol) and (−)cinchonidine (7.95 g, 27.04 mmol) in methyl-tert-butyl ether (100 mL) heated on a steam-bath was added n-heptane (200 mL). The mixture was heated on the steam bath to boiling and allowed to cool for 4 h during which time crystals formed. Filtration of the suspension yielded a crystalline solid (18.7 g). This solid was dissolved in 2-butanone (30 mL) followed by the addition of n-heptane (500 mL). After standing for 16 hours, the resulting suspension was filtered yielded a white solid (10.3 g). This solid was dissolved in ethyl acetate (150 mL), washed with 1 N hydrochloric acid (100 mL) and brine (2×50 mL), dried over MgSO₄, filtered, and concentrated in vacuo yielding a viscous yellow oil (5.2 g, 59%): ¹H NMR (acetone-d6/300 MHz) 7.16 (s, 1H), 6.77 (d, 1H, J=2.7 Hz), 6.94 (d, 1H, J=8.7 Hz), 6.64 (m, 1H), 6.39 (d, 1H, J=8.7 Hz) 5.13 (q, 1H, J=7.2 Hz). Anal. Calc'd for C₁₂H₆O₄F₆: C, 43.92; H, 1.84; N, 0.0. Found: C, 43.79; H, 1.83; N, 0.0. This compound was determined to have an optical purity of greater than 90% ee. Chiral purity was determined as describe in Example 2.

### Example 7

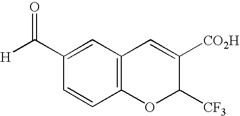

6-Formyl-2-(trifluoromethyl)-2H-1-benzopyran-3-carboxylic Acid

Step 1. Preparation of Ethyl 6-formyl-2-(trifluoromethyl)-2H-1-benzopyran-3-carboxylate.

A 50 mL round bottom flask was charged with 5-formylsalicylaldehyde (3.21 g, 21.39 mmol), ethyl 4,4,4-trifluorocrotonate (3.50 mL, 3.96 g, 23.53 mmol), dimethylformamide (15 mL) and potassium carbonate (2.95 g, 21.39 mmol) and heated to 60° C. for 12 hours. Additional ethyl 4,4,4-trifluorocrotonate (3.50 mL, 3.96 g, 23.53 mmol) was added and the reaction heated for 16 hours at 75° C. After cooling to room temperature, the reaction was partitioned between H₂O and diethyl ether. The organic phase was washed with saturated NaHCO₃ solution, KHSO₄ solution (0.25 M), brine, treated with decolorizing carbon (warmed gently). The resulting black suspension was dried over MgSO₄, vacuum filtered through diatomaceous earth, and concentrated in vacuo yielding an orange crystalline mass. This material was recrystallized from hot hexanes yielding the ester (1.51 g, 24%) as orange crystals: mp 84.3-86.2° C. ¹H NMR (acetone-d6/300 MHz) 9.96 (s, 1H), 8.06 (d, 1H, J=2 Hz), 8.02 (s, 1H), 7.99 (dd, 1H, J=8.5, 2.0 Hz), 7.24 (d, 1H, J=8.5 Hz), 5.99 (q, 1H, J=7.1 Hz), 4.43-4.25 (m, 2H), 1.34 (t, 3H, J=7.3 Hz). FABLRMS m/z 301 (M+H). EIHRMS m/z 300.0605 (M+, Calc'd 300.0609). Anal. Calc'd for C14H11F3O4: C, 56.01; H, 3.69. Found: C, 56.11; H, 3.73.

Step 2. Preparation of 6-formyl-2-(trifluoromethyl)-2H-1-benzopyran-3-carboxylic Acid.

The ester (Step 1) was converted to the acid via a method similar to that described in Example 1, Step 2: mp 211.3-215.7° C. 1H NMR (acetone-d6/300 MHz) 9.97 (s, 1H), 8.07 (d, 1H, J=2.0 Hz), 8.03 (s, 1H), 8.00 (dd, 1H, J=8.3, 2.0 Hz), 7.25 (d, 1H, J=8.5 Hz), 5.98 (q, 1H, J=6.9 Hz). FABLRMS m/z 273 (M+H). EIHRMS m/z 272.0266 (M+, Calc'd 272.0296). Anal. Calc'd for C12H7F3O4: C, 52.95; H, 2.59. Found: C, 52.62; H, 2.58.

### Example 8

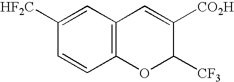

### 6-(Difluoromethyl)-2-(trifluoromethyl)-2H-1-benzopyran-3-carboxylic Acid

Step 1. Preparation of Ethyl 6-(difluoromethyl)-2-(trifluoromethyl)-2H-1-benzopyran-3-carboxylate.

Ethyl 6-formyl-2-trifluoromethyl-2H-1-benzopyran-3-carboxylate (Example 7, Step 1)(1.672 g, 5.569 mmol) in methylene chloride (1.5 mL) was added to methylene chloride (1.5 mL) and diethylaminosulfur trifluoride (DAST) (0.74 mL, 0.898 g, 5.569 mmol) over 0.07 hours via syringe. After stirring for 20 hours the reaction was poured into aqueous HCl (2.0 N) and the mixture was extracted with diethyl ether. The ethereal phase was washed with dilute aqueous HCl (2.0 N), saturated NaHCO₃ solution, brine, dried over MgSO₄, filtered and concentrated in vacuo yielding a clear colorless oil. This oil was purified by flash chromatography (Silica gel 60, Eluant (5:1; Hexanes:Ethyl Acetate) yielding ethyl 6-difluoromethyl-2-trifluoromethyl-2H-1-benzopyran-3-carboxylate (0.96 g, 54%) as an oil which solidified upon standing. This product was of sufficient purity to be used in the next step without further purification: ¹H NMR (acetone-d6/300 MHz) 7.97 (s, 1H), 7.74 (s, 1H), 7.65 (d, 1H, J=8.5 Hz), 7.18 (d, 1H, J=8.5 Hz), 6.90 (t, 1H, J=56.0 Hz), 5.94 (q, 1H, J=7.0 Hz), 4.40-4.25 (m, 2H), 1.34 (t, 3H, J=7.0 Hz).

Step 2. Preparation of 6-(difluoromethyl)-2-(trifluoromethyl)-2H-1-benzopyran-3-carboxylic Acid.

Aqueous NaOH (1.31 mL, 3.277 mmol, 2.5 M solution) was added in one portion to the ester (Step 1)(0.880 g, 2.731 mmol) in THF:EtOH:H₂O (7:2:1, 10 mL). The resulting solution was stirred for 60 hours. The reaction mixture was partially concentrated in vacuo to remove the organic solvents and was diluted with H₂O. The resulting aqueous solution was washed with diethyl ether, sparged with nitrogen to remove trace ether, and acidified with concentrated HCl. The resulting oily suspension was extracted with diethyl ether. The combined organic phases were dried over MgSO₄, filtered and concentrated in vacuo yielding the title compound (0.483 g, 60%) as an oil which solidified as a white crystalline mass: mp 134.7-136.2° C. 1H NMR (acetone-d6/300 MHz) 7.97 (s, 1H), 7.73 (s, 1H), 7.67 (dd, 1H, J=8.5, 1.0 Hz), 7.17 (d, 1H, J=8.5 Hz), 6.89(t, 1H, J=56.2 Hz), 5.90 (q, 1H, J=7.1 Hz). FAB-ESLRMS m/z 293 (M−H). EIHRMS m/z 293.0235 (M−H, Calc'd 293.0237). Anal. Calc'd for C12H7F5O3: C, 49.00; H, 2.40. Found: C, 48.78; H,2.21.

### Example 9

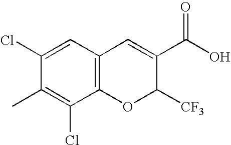

### 6,8-Dichloro-7-methyl-2-(trifluoromethyl)-2H-1-benzopyran-3-carboxylic Acid

Step 1. Preparation of 3,5-dichloro-4-methylsalicylaldehyde.

2,4-Dichloro-3-methylphenol (25.0 g, 141.2 mmol) was added to methanesulfonic acid (100 mL). With stirring, hexamethylenetetramine (HMTA) (39.8 g, 282.4 mmol) and additional methanesulfonic acid (100 mL) was added portion-wise during which time the reaction began to froth and exotherm. The resulting mixture was heated to 100° C. for 3 hours. The crude ocher colored suspension was cooled to 50° C. and poured over a mechanically stirred mixture of ice-water (2 L). A yellow precipitate was formed which was collected by vacuum filtration. This solid was purified by flash chromatography (silica, hexanes-methylene chloride, 9:10) yielding the salicylaldehyde as a pale yellow powder (6.17 g, 21%; mp 94.0-95.1° C.) of suitable purity to use without further purification.

Step 2. Preparation of ethyl 6,8-dichloro-7-methyl-2-(trifluoromethyl)-2H-1-benzopyran-3-carboxylate.

A mixture of 3,5-dichloro-4-methylsalicylaldehyde (Step 1)(5.94 g, 29.0 mmol) and ethyl 4,4,4-trifluorocrotonate (7.67 g, 45.6 mmol) dissolved in anhydrous DMSO (10 mL) was treated with triethylamine (5.88 g, 58.1 mmol). The reaction was stirred at 85° C. for 49 hours then cooled in ice and filtered to give an orange solid. The solid was dissolved in ethyl acetate (100 mL), washed with 3 N HCl (2×50 mL), saturated NaHCO₃, washed with brine, dried over MgSO₄, and concentrated in vacuo to give a yellow solid (8.63 g, 84%): mp 117.1-119.5° C. ¹H NMR (CDCl₃/300 MHz) 7.63 (s, 1H), 7.17 (s, 1H), 5.80 (q, 1H, J=6.6 Hz), 4.33 (m, 2H), 2.48 (s, 3H), 1.35 (t, 3H, J=7.1 Hz).

Step 3. Preparation of 6,8-dichloro-7-methyl-2-(trifluoromethyl)-2H-1-benzopyran-3-carboxylic Acid.

The ester from Step 2 (8.39 g 23.6 mmol) was dissolved in THF (30 mL) and ethanol (20 mL), treated with 2.5 N sodium hydroxide (20 mL, 50 mmol), and stirred at room temperature for 3.5 hours. The reaction mixture was concentrated in vacuo, acidified with 3 N HCl, filtered, and recrystallized from ethanol/water to yield a yellow solid (6.0 g, 78%): mp 229.9-230.9° C. ¹H NMR (acetone-d6/300 MHz) 7.90 (s, 1H), 7.58 (s, 1H), 6.00 (q, 1H, J=6.8 Hz), 2.50 (s, 3H). FABLRMS m/z 325 (M−H). FABHRMS m/z 324.9636 (M−H, Calc'd 324.9646). Anal. Calc'd for C₁₂H₇Cl₂F₃O₃: C, 44.07; H, 2.16; Cl, 21.68. Found: C, 44.06; H, 2.21; Cl, 21.74.

### Example 10

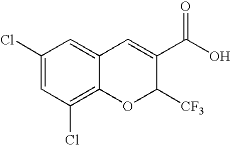

### 6,8-Dichloro-2-trifluoromethyl-2H-1-benzopyran-3-carboxylic Acid

3,5-Dichlorosalicylaldehyde was converted to the title compound by a procedure similar to that described in Example 9, Steps 2 & 3: mp 212.8-216.8° C. ¹H NMR (CDCl3/300 MHz) 7.77 (s, 1H), 7.41 (d, 1H, J=2.4 Hz), 7.18 (d, 1H, J=2.2 Hz), 5.82 (q, 1H, J=6.7 Hz). FABLRMS m/z 311 (M−H). FABHRMS m/z 312.9644 (M+H, Calc'd 312.9646). Anal. Calc'd for C11H5F3Cl2O3: C, 42.20; H, 1.61. Found: C, 42.50; H, 1.71.

### Example 11

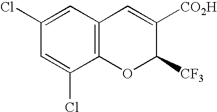

### (S)-6,8-Dichloro-2-(trifluoromethyl)-2H-1-benzopyran-3-carboxylic Acid

6,8-Dichloro-2-(trifluoromethyl)-2H-1-benzopyran-3-carboxylic acid (Example 10)(300 g, 1.04 mol) was added to ethyl acetate (750 mL). The mixture was stirred for 5 minutes, warmed to 70° C. and held at this temperature for 5 minutes. The resulting solution was cooled to 50° C. and (s)-(−)-α-methylbenzylamine (58 g, 0.48 mol)was added. Heptane (1880 mL) was added and the mixture stirred for 0.5 hour, then stirring was discontinued. The reaction was allowed to cool to 22° C. and stand for 8 hours. The salt crystallized during this time and was collected by vacuum filtration. The solid was washed with ethyl acetate-heptane (1:3, 2×50 mL). The solid obtained was dried at 40° C. under vacuum (20 mm) for 24 hours to give the salt (35 g, 16%).

A three-neck 2 L round bottom flask was purged with nitrogen and was charged with deionized water (750 mL) and the salt (103 g, 0.24 mole; This material was obtained using a similar procedure to that described above). To the resulting stirred suspension was added concentrated HCl (37 mL) drop-wise over 0.5 hours with good stirring below 20° C. causing the free carboxylic acid to precipitate. After stirring for 2 hours, the suspension was vacuum filtered and the solid washed with deionized water (5×50 mL; until the washings were neutral). The solid was dried at 40° C. under vacuum (20 mm) for 12 hours yielding the title compound as a solid (74 g, 100%): mp 166.0-168.4° C. ¹H NMR (acetone-d6/300 MHz) 7.94 (s, 1H), 7.60 (s, 2H), 6.04 (q, 1H, J=6.8 Hz). ESHRMS m/z 310.9489 (M−H, Calc'd 310.9450). This compound was determined to have an optical purity of greater than 90% ee. The optical purity was determined by the method described in Example 2.

### Example 12

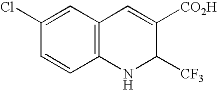

### 6-Chloro-1,2-dihydro-2-(trifluoromethyl)-3-quinolinecarboxylic Acid

Step 1. Preparation of 2-amino-5-chlorobenzaldehyde.

2-Amino-5-chlorobenzyl alcohol (4.8 g, 30 mmol) and activated manganese (IV) oxide (21 g, 240 mmol) were refluxed in chloroform (100 mL) for 1 hour. The contents were allowed to cool, filtered through diatomaceous earth and concentrated in vacuo to afford the 2-amino-5-chlorobenzaldehyde as a dark solid (4.14 g, 81%): mp 74-76° C. ¹H NMR (CDCl₃, 300 MHz) 9.80 (s, 1H), 7.42 (s, 1H), 7.23 (d, 1H, J=7.0 Hz), 6.60 (d, 1H, J=7.0 Hz).

Step 2. Preparation of Ethyl 6-chloro-1,2-dihydro-2-(trifluoromethyl)-3-quinolinecarboxylate.

The 2-amino-5-chlorobenzaldehyde from Step 1 (15.0 g, 96 mmol), anhydrous potassium carbonate (27.6 g, 200 mmol), and ethyl 4,4,4-trifluorocrotonate (34 mL, 200 mmol) were mixed in anhydrous dimethylormamide (60 mL) and heated at 100° C. for 7 hours. The contents were allowed to cool and partitioned between ethyl acetate (200 mL) and water (200 mL). The aqueous layer was extracted with ethyl acetate (1×100 mL). The ethyl acetate extracts were combined and washed with brine (1×200 mL), dried over MgSO₄, and concentrated in vacuo leaving a dark oil which solidified upon standing. The solid was purified by flash chromatography (silica gel; ethyl acetate-hexanes, 1:9). Fractions containing the desired product were combined, concentrated in vacuo and the residue recrystallized from ethyl acetate-hexanes to afford the ethyl 6-chloro-1,2-dihydro-2-(trifluoromethyl)-3-quinolinecarboxylate as a yellow solid (16.36 g, 56%): mp 132.6-134.2° C. ¹H NMR (CDCl₃, 300 MHz) 7.61 (s, 1H), 7.10 (m, 2H), 6.55 (d, 1H, J=8.0 Hz), 5.10 (q, 1H, J=6.0 Hz), 4.55 (brs, 1H), 4.23 (m, 2H), 1.32 (t, 3H, J=7.0 Hz). FABHRMS m/z 306.0468 (M+H⁺, Calc'd 306.0509). Anal. Calc'd for C₁₃H₁₁NO₂F₃Cl: C, 51.08; H, 3.63; N, 4.58. Found: C, 50.81; H, 3.49; N, 4.72.

Step 3. Preparation of 6-chloro-1,2-dihydro-2-(trifluoro-methyl)-3-quinolinecarboxylic Acid.

The ester from Step 2 (1.7 g, 5.6 mmol) and 2.5 N sodium hydroxide (4.4 mL, 11 mmol) were mixed in tetrahydrofuran (25 mL), methanol (10 mL), and water (25 mL). After stirring overnight, contents were concentrated in vacuo to remove the THF and methanol. The aqueous solution remaining was extracted with diethyl ether (2×100 mL). The resulting aqueous layer was acidified with 2 N HCl causing the precipitation of an oil. The oil was purified by flash chromatography on silica gel, eluting with ethyl acetate-hexanes (1:1). Fractions containing the desired product were combined, and concentrated in vacuo. The residue was triturated with dichloromethane, and filtered to afford the 6-chloro-1,2-dihydro-2-(trifluoromethyl)-3-quinolinecarboxylic acid as a yellow solid (0.645 g, 41%): mp 187.8-188.8° C. ¹H NMR (acetone-d₆, 300 MHz) 7.69 (s, 1H), 7.36 (s, 1H), 7.15 (d, 1H, J=8.0 Hz), 6.83 (d, 1H, J=8.0 Hz), 6.60 (brs, 1H), 5.20 (m, 1H). ESHRMS m/z 276.0040 (M−H,Calc'd 276.0039). Anal. Calc'd for C₁₁H₇NO₂F₃Cl+2.6% H₂O: C, 46.39; H, 2.98; N, 4.92. Found: C, 45.99; H, 2.54; N, 4.85.

### Example 13

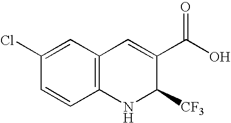

### (S)-6-chloro-1,2-dihydro-2-(trifluoromethyl)-3-quinolinecarboxylic Acid

To a solution of 6-chloro-1,2-dihydro-2-(trifluoromethyl)-3-quinolinecarboxylic acid (Example 12)(6.75 g, 24.3 mmol) in ethyl acetate (25 mL) was added (S)-(−)-a-methylbenzylamine (1.50 g, 12.2 mmol). To the resulting solution was added hexanes (50 mL) with mixing. Stirring was discontinued and the reaction held static at room temperature for 16 hours during which time yellow crystals formed. The crystals were collected and washed with ethyl acetate-hexanes (100 mL, 1:2). The resulting yellow solid (932 mg) was dissolved in ethyl acetate (20 mL) and extracted with 1 N HCl (3×10 mL). The organic layer was dried over sodium sulfate and solvent removed at reduced pressure. The (s)-6-chloro-1,2-dihydro-2-(trifluoromethyl)-3-quinolinecarboxylic acid was obtained as a yellow solid (648 mg, 10% yield). mp 173-176° C. ¹H NMR (acetone-d₆, 300 MHz) 7.80 (s, 1H), 7.35(d, 1H, J=2.2 Hz), 7.18 (d, 1H, J=8.0, J=2.2 Hz), 6.86 (d, 1H, J=8.0 Hz), 6.60 (brs, 1H), 5.20 (m, 1H). Anal. Calc'd. for C₁₁H₇NO₂F₃Cl C, 47.40H, 2.54; N, 5.40. Found C, 47.49; H, 2.60; N, 4.98. The compound was determined to have an optical purity greater than 90% ee. Optical purity was determined by HPLC as described in Example 2.

### Example 14

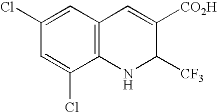

### 6,8-Dichloro-1,2-dihydro-2-(trifluoromethyl)-3-quinolinecarboxylic Acid

The 1,2-dihydro-3-quinolinecarboxylic acid was prepared by a procedure similar to that described in Example 12: mp 223.4-225.7° C. ¹H NMR (acetone-d₆, 300 MHz) 7.82 (s, 1H), 7.40 (m, 2H), 6.53 (brs, 1H), 5.40 (m, 1H). ESHRMS m/z 309.9657 (M−H, Calc'd 309.9649). Anal. Calc'd for C₁₁H₆NO₂F₃Cl₂: C, 42.34; H, 1.94; N, 4.49. Found: C, 42.20; H, 1.74; N, 4.52.

### Example 15

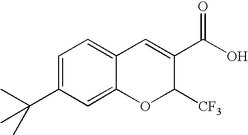

### 7-(1,1-Dimethylethyl)-2-trifluoromethyl-2H-1-benzopyran-3-carboxylic Acid

Ethyl 7-(1,1-dimethylethyl)-2-(trifluoromethyl)-2H-1-benzopyran-3-carboxylate (Example 3, Step 2) was hydrolyzed to the carboxylic acid via a procedure similar to that described in Example 1, Step 2: mp 165.6-166.8° C. ¹H NMR (acetone-d6/300 MHz) 7.86 (s, 1H), 7.38 (d, 1H, J=8.1 Hz), 7.15 (dd, 1H, J=1.8 Hz, and J=7.8 Hz), 7.05 (bs, 1H), 5.79 (q H−F, 1H, J=7.2 Hz), 1.32 (s, 9H). FABHRMS m/z 301.1033 (M+H, Calc'd 301.1051). Anal. Calc'd for C15H15F3O3: C, 60.00; H, 5.04. Found: C, 59.80; H, 5.10.

### Example 16

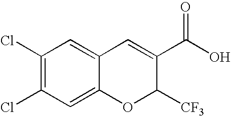

### 6,7-Dichloro-2-trifluoromethyl-2H-1-benzopyran-3-carboxylic Acid

3,4-Dichlorophenol was converted to the title compound by a procedure similar to that described in Example 2: mp 196.1-198.3° C. ¹H NMR (acetone-d6/300 MHz) 7.90 (s, 1H), 7.74 (s, 1H), 7.30 (s, 1H), 5.88 (q, 1H, J=6.9 Hz). FABLRMS m/z 314 (M+H). Anal. Calc'd for C11H5Cl2F3O3: C, 42.20; H, 1.61. Found: C, 42.31; H, 1.65.

### Example 17

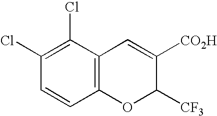

### 5,6-Dichloro-2-(trifluoromethyl)-2H-1-benzopyran-3-carboxylic Acid

5,6-Dichlorosalicylaldehyde was prepared by the procedure described in Cragoe, E. J.; Schultz, E. M., U.S. Pat. No. 3,794,734, 1974. This salicylaldehyde was converted to the title compound by a similar procedure to that described in Example 1: mp 211.5-213.5° C. 1H NMR (acetone-d6/300 MHz) 8.09 (s, 1H), 7.63 (d, 1H, J=8.9 Hz), 7.12 (d, 1H, J=8.9 Hz), 5.94 (q, 1H, J=7.0 Hz). ESLRMS m/z 311 (M−H). EIHRMS m/z 311.9583 (M+, Calc'd 311.9568). Anal. Calc'd for C11H5Cl2F3O3: C, 42.20; H, 1.61. Found: C, 42.33; H, 1.67.

### Example 18

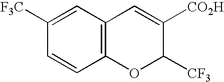

### 2,6-Bis(trifluoromethyl)-2H-1-benzopyran-3-carboxylic Acid

Step 1. Preparation of Ethyl 2,6-bis(trifluoromethyl)-4-oxo-4H-1-benzopyran-3-carboxylate.

To a stirred solution of ethyl 4,4,4-trifluoroacetoacetate (3.22 mL, 4.06 g, 22.07 mmol) in toluene (100 mL) was added portion-wise sodium hydride (0.971 g, of 60% oil dispersion reagent, 22.07 mmol) causing gas evolution. After gas evolution has subsided, 2-fluoro-5-(trifluoromethyl)benzoyl chloride (5.00 g, 22.07 mmol) was added. The reaction was stirred at room temperature for 24 hours, then heated to 105° C. for 24 hours. After cooling to room temperature, the reaction was diluted with diethyl ether and the resulting solution was washed with H₂O and brine, dried over MgSO₄, filtered and concentrated in vacuo yielding a slightly sticky white solid. This solid was triturated with hexanes yielding the desired ester (3.05 g, 39%) as a white powder: mp 116-120.1° C. 1H NMR (CDCl₃/300 MHz) 8.52 (d, 2H, J=1.6 Hz), 8.03 (dd, 1H, J=8.9, 2.2 Hz), 7.71 (d, 1H, J=8.9 Hz), 4.48 (q, 2H, J=7.3 Hz), 1.39 (t, 3H, J=7.3 Hz). FABLRMS m/z 355 (M+H). Anal. Calc'd for C₁₄H₈F₆₀₄: C, 47.45; H, 2.28. Found: C, 47.59; H, 2.43.

Step 2. Preparation of ethyl 2,6-bis(trifluoromethyl)-4-oxo-dihydrobenzopyran-3-carboxylate.

A 250 mL round bottom flask was charged with ethyl 2,6-bis(trifluoromethyl)-benzopyran-4-one-3-carboxylate (Step 1)(2.307 g, 6.513 mmol) and THF (20 mL) yielding a pale yellow solution. Ethanol (20 mL) was added and the reaction chilled in an ice-salt bath. While maintaining the reaction temperature at below 9° C., NaBH₄ (0.246 g, 6.513 mmol) was added in two portions and the mixture stirred 1 h. The crude reaction mixture was poured into a vigorously stirred mixture of ice (200 mL) and concentrated HCl (12 N, 5 mL) yielding a precipitate. Vacuum filtration of the resulting suspension yielded the desired keto ester (2.204 g, 87%) as faint pink powder of suitable purity to use in the next step without further purification: mp 71.8-76.9° C. ¹H NMR (acetone-d6/300 MHz) 12.71 (br s, 1H exch), 8.01 (d, 1H, J=2.0 Hz), 8.01 (d, 1H, J=2.0 Hz), 7.88 (dd, 1H, J=8.7, 1.8 Hz), 7.31 (d, 1H, J=8.7 Hz), 5.98 (q, 1H, J=6.6 Hz), 4.51-4.28 (m, 2H), 1.35 (t, 3H, J=7.0 Hz). FABLRMS m/z 355 (M−H). ESHRMS m/z 355.0394 (M−H, Calc'd 355.0405).

Anal. Calc'd for C14H10F6O4: C, 47.21; H, 2.83.

Found: C, 47.31; H, 2.97.

Step 3. Preparation of Ethyl 2,6-bis(trifluoromethyl)-4-trifluoromethanesulfonato-2H-1-benzopyran-3-carboxylate.

A 50 mL 3-neck Morton flask fitted with addition funnel, 2 stoppers was charged with 2.6-di-tert-butylpyridine (1.576 g, 1.50 mmol), methylene chloride (12 mL), and then via syringe was added trifluoromethanesulfonic anhydride (1.08 mL, 1.80 g, 1.25 mmol). To this solution was added dropwise a solution the keto ester (Step 2) (1.822 g, 5.115 mmol) in methylene chloride (10 mL) over 0.33 h and the reaction stirred for 48 h. The resulting off-white suspension was transferred to a 100 mL round bottom flask and was concentrated in vacuo. The residue was suspended in diethyl ether (50 mL) and vacuum filtered to remove salts. The filtrate was further diluted with diethyl ether (50 mL) and was washed with ice cold HCl solution (2 N), brine, and dried over Na₂CO₃, filtered and concentrated in vacuo yielding the desired triflate (1.64 g, 66%) as a tan clumpy powder of suitable purity to use in the next step without further purification.

Step 4. Preparation of Ethyl 2,6-bis(trifluoromethyl)-2H-1-benzopyran-3-carboxylate.

A 25 mL pear flask was charged with LiCl (0.136 g, 3.219 mmol), affixed to a high vacuum line and heated with a heat gun removing superficial water. The flask was allowed to cool to room temperature, and tetrakis(triphenylphosphine)palladium(0)(0.124 g, 0.107 mmol) and THF (2 mL) were added. A reflux condenser was affixed to the flask and the apparatus was purged with nitrogen. A solution of the triflate(Step 3)(0.524 g, 1.073 mmol)in THF (2 mL) and tri-n-butyltin hydride (0.32 mL, 0.34 g, 1.18 mmol) were added sequentially via syringe. The resulting light orange solution was heated to 50° C. with stirring for 1 h, 60° C. for one hour, and 65° C. for one hour. The reaction was allowed to cool to room temperature and was poured into 2 N HCl, stirred, and extracted with hexanes. The hexane phase was dried over MgSO₄, filtered and concentrated yielding a light brown oil. The oil was dissolved in hexane and was washed with aqueous ammonium fluoride solution. The resulting hexane phase was dried over MgSO₄, filtered and concentrated in vacuo yielding a dull yellow oily solid which solidified as a flaky powder (0.443 g). This solid was purified by flash silica chromatography (eluant: hexanes-methylene chloride, 4:1) yielding ethyl 2,6-di-trifluoromethyl-2H-1-benzopyran-3-carboxylate(0.069 g, 19%) as a white crystalline solid of suitable purity to proceed with the next step.

Step 5. Preparation of 2,6-bis(trifluoromethyl)-2H-1-benzopyran-3-carboxylic Acid.

To a stirred solution of the ester (Step 4) (0.065 g, 0.191 mmol) in THF-EtOH—H₂O (7:2:1, 1 mL) was added NaOH solution (0.084 mL, 0.210 mmol)in one portion at room temperature and allowed to stir overnight. The reaction was partially concentrated in vacuo yielding a pale yellow clear syrup. The syrup was diluted with water (5 mL) and brine (1 mL) and was washed with diethyl ether (3×5 mL). The resulting aqueous phase was sparged with nitrogen to remove trace ether. With stirring, concentrated HCl was added to the aqueous phase causing the formation of a very fine white precipitate. This suspension was extracted with diethyl ether and the ether dried over Na₂SO₄, filtered, and concentrated by slow evaporation at atmospheric pressure. The resulting product was recrystallized from hexanes and ethyl acetate yielding the title compound (0.038 g, 64%) as a fine tan powder: mp 143.5-145.2° C. ¹H NMR (acetone-d6/300 MHz) 11.97-11.67 (br s, 1H), 8.03 (s, 1H), 7.92 (s, 1H), 7.77 (d, 1H, J=8.5 Hz), 7.26 (d, 1H, J=8.7 Hz), 5.96 (q, 1H, J=7.0 Hz). FABLRMS m/z 311 (M−H). ESHRMS m/z 311.0107 (M−H, Calc'd 311.0143).

### Example 19

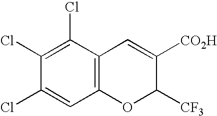

### 5,6,7-Trichloro-2-(trifluoromethyl)-2H-1-benzopyran-3-carboxylic Acid

3,4,5-Trichlorophenol was converted to 4,5,6-trichlorosalicylaldehyde via a procedure similar to that described in Example 9, Step 1. The 4,5,6-trichirorsalicylaldehyde was converted to the title compound by a procedure similar to that described in Example 1: mp 236.2-239.3° C. ¹H NMR (acetone-d6/300 MHz) 8.05 (s, 1H), 7.40 (s, 1H), 5.99 (q, 1H, J=7.0 Hz). ESLRMS m/z 345 (M−H). ESHRMS m/z 344.9113 (M−H, Calc'd 344.9100). Anal. Calc'd for C11H4Cl3F3O3+0.89 wt % H₂O: C, 37.68; H, 1.25; Cl, 30.33. Found: C, 37.48; H, 1.25; Cl, 30.33.

### Example 20

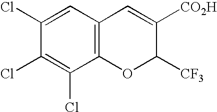

### 6,7,8-Trichloro-2-(trifluoromethyl)-2H-1-benzopyran-3-carboxylic Acid

2,3,4-Trichlorophenol was converted to 3,4,5-trichlorosalicylaldehyde via a procedure similar to that described in Example 9, Step 1. The 3,4,5-trichirorsalicylaldehyde was converted to the title compound by a procedure similar to that described in Example 1: mp 222.0-225.3° C. ¹H NMR (acetone-d6/300 MHz) 7.94 (s, 1H), 7.78 (s, 1H), 6.07 (q, 1H, J=7.0 Hz). ESLRMS m/z 345 (M−H). EIHRMS m/z 344.9117 (M−H, Calc'd 344.9100). Anal. Calc'd for C11H4Cl3F3O3+1.56 wt % H₂O: C, 37.43; H, 1.32; Cl, 30.13. Found: C, 37.79; H,0.93; Cl, 29.55.

### Example 21

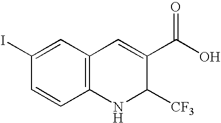

### 6-Iodo-1,2-dihydro-2-(trifluoromethyl)-3-quinolinecarboxylic Acid

Step 1. Preparation of ethyl 6-iodo-1,2-dihydro-2-(trifluoromethyl)-3-quinolinecarboxylate.

A mixture of 5-iodo-2-aminobenzaldehyde (24.0 g, 96.7 mmol), diazbicyclo[2.2.2]-undec-7-ene (32.2 g, 212.0 mmol), and ethyl 4,4,4-trifluorocrotonate (35.7 g, 212.0 mmol) in 1,3-dimethyl-3,4,5,6-tetrahydro-2(1H)-pyrimidinone (48 mL) was heated at 60° C. for 8 hours. The solution was cooled to room temperature and the solution poured into ethyl acetate-hexanes (1:1, 500 mL). The solution was extracted with 2.5 N aqueous hydrochloric acid (2×200 mL), saturated aqueous ammonium chloride (2×200 mL), dried over sodium sulfate, filtered and concentrated in vacuo. The resulting dark yellow oil was dissolved in hexanes (100 mL) and fine yellow crystals formed upon standing. Vacuum filtration of this suspension yielded ethyl 6-iodo-1,2-dihydro-2-(trifluoromethyl)-3-quinolinecarboxylate as fine yellow crystals (19.3 g, 50% yield): mp 137-138° C. ¹H NMR (CDCl₃, 300 MHz) 7.62 (s, 1H), 7.36-7.48 (m, 2H), 6.43 (d, J=8.2 Hz), 5.36 (brs, 1H), 5.11 (q, 1H, J=7.1 Hz), 4.25-4.35 (m, 2H), 1.34 (t, 3H, J=7.0 Hz). ESHRMS m/z 395.9716 (M−H, Calc'd 395.9708).

Step 2. Preparation of 6-iodo-1,2-dihydro-2-(trifluoromethyl)-3-quinolinecarboxylic Acid

Hydrolysis of the ester (Step 1) was performed by a procedure similar to that described in Example 12, Step 3, yielding the carboxylic acid. mp 188-192° C. ¹H NMR (CD₃OD/300 MHz) 7.668 (s, 1H), 7.46 (d, 1H, J=2.2 Hz), 7.39 (dd, 1H, J=8.4, 2.2 Hz), 6.52 (d, 1H, J=8.4 Hz), 5.01 (q, 1H, J=7.5 Hz). ESHRMS m/z 367.9401 (M, Calc'd 367.9395).

### Example 22

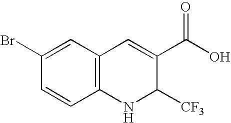

### 6-Bromo-1,2-dihydro-2-(trifluoromethyl)-3-quinolinecarboxylic Acid

The 1,2-dihydro-3-quinolinecarboxylic acid was prepared by a procedure similar to that described in Example 21: mp 185-186° C. ¹H NMR (CD₃OD/300 MHz) 7.68 (s, 1H), 7.31 (d, 1H, J=2.2 Hz), 7.23 (dd, 1H, J=8.7, 2.2 Hz), 6.64 (d, 1H, J=8.7 Hz), 5.01 (q, 1H, J=7.5 Hz). EIHRMS m/z 319.9519 (M, Calc'd 319.9534). Anal. Calc'd for C₁₁H₇BrF₃NO₂: C, 41.02; H, 2.19; N, 4.35; Found: C, 41.27, H, 2.23, N, 4.26.

### Example 23

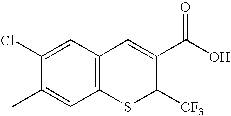

### 6-Chloro-7-methyl-2-(trifluoromethyl)-2H-1-benzothiopyran-3-carboxylic Acid

Step 1. Preparation of N,N-dimethyl-O-(4-chloro-2-formyl-5-methylphenyl)thiocarbamate.

A mixture of 5-chloro-4-methylsalicylaldehyde (12.96 g, 76.0 mmol) and triethylamine (11.58 g, 114.4 mmol) was dissolved in anhydrous DMF (15 mL) treated with N,N-dimethylthiocarbamoyl chloride (11.25 g, 91.0 mmol) and stirred at room temperature for 16 hours. The reaction was treated with 3 N HCl (50 mL) and filtered to give an orange solid. The solid was dissolved in ethyl acetate washed with 3 N HCl, water, brine, dried over anhydrous MgSO4, filtered and concentrated in vacuo to afford a brown solid (16.79 g) which was recrystallized from diethyl ether/hexane to give the O-aryl thiocarbamate as a tan solid (4.92 g, 25%): ¹H NMR (acetone-d6/300 MHz) 9.96 (s, 1H), 7.80 (s, 1H), 7.19 (s, 1H), 3.46 (s, 3H), 3.42 (s, 3H), 2.43 (s, 3H).

Step 2. Preparation of N,N-dimethyl-S-(4-chloro-2-formyl-5-methylphenyl)thiocarbamate.

The O-aryl thiocarbamate (Step 1) (4.92 g, 19.1 mmol) was dissolved in N,N-dimethylaniline (25 mL) and immersed in and stirred at 200° C. for 1.5 hours. The reaction mixture was cooled to room temperature and poured into a mixture of 3 N HCl (200 mL) and ice. Filtration gave a brown semisolid which was dissolved in ethyl acetate, washed with 3 N HCl, brine, dried over anhydrous MgSO4, filtered and concentrated in vacuo to afford the S-arylthiocarbamate as a brown oil (3.80 g, 77%) which was used in the next step without further purification.

Step 3. Preparation of Ethyl 6-chloro-7-methyl-2-(trifluoromethyl)-2H-1-benzothiopyran-3-carboxylate.

The S-arylthiocarbamate (Step 2) (3.80 g, 14.7 mmol) was dissolved in THF (10 mL) and ethanol (10 mL), treated with 2.5 N sodium hydroxide (16.5 mL, 34.2 mmol), and stirred at room temperature for 0.9 hours. The reaction was diluted with diethyl ether and washed with 3 N HCl, brine, dried over MgSO₄, filtered and concentrated in vacuo to yield the crude substituted 2-mercaptobenzaldehyde as a brown oil (2.82 g). This oil was added to DMF (10 mL) and ethyl 4,4,4-trifluorocrotonate (3.89 g, 23.1 mmol). With stirring, K₂CO₃ (3.23 g, 23.4 mmol) was added causing the reaction to become a deep red. The reaction was stirred at room temperature for 14.5 hours, acidified with 3 N HCl, extracted with ethyl acetate. The resulting organic phase was washed with brine, dried over MgSO₄, filtered and concentrated in vacuo to give a yellow solid (6.36 g) which was used in the next step without further purification.

Step 4. Preparation of 6-chloro-7-methyl-2-(trifluoromethyl)-2H-1-benzothiopyran-3-carboxylic Acid

The ester from Step 3 (2.02 g, 6.0 mmol) was dissolved in THF (10 mL) and ethanol (10 mL), treated with 2.5 N sodium hydroxide (5.5 mL, 13.8 mmol), and stirred at room temperature for 4.8 hours. The reaction mixture was concentrated in vacuo, acidified with 3 N HCl yielding a suspension. The solid was collected by filtration and was recrystallized from ethanol-water to yield the title compound as a yellow solid (0.20 g, 11%): mp 240.5-241.7° C. ¹H NMR (acetone-d6/300 MHz) 7.99 (s, 1H), 7.67 (s, 1H), 7.43 (s, 1H), 4.99 (q, 1H, J=8.5 Hz), 2.39 (s, 3H). FABLRMS m/z 307 (M−H). FABHRMS m/z 306.9831 (M−H, Calc'd 306.9807). Anal. Calc'd for C₁₂H₈ClF₃O₂S: C, 46.69; H, 2.61; Cl, 11.48. Found: C, 46.78; H, 2.61; Cl, 11.41.

### Example 24

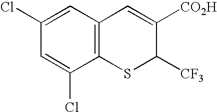

### 6,8-Dichloro-2-trifluoromethyl-2H-1-benzothiopyran-3-carboxylic Acid

The 2H-1-benzothiopyran-3-carboxylic acid was prepared by a procedure similar to the method described in Example 23: mp 217.9-220.3° C. ¹H NMR (acetone-d6/300 MHz) 12.50-11.20 (br s, 1H exch.), 8.06 (s, 1H), 7.75 (d, 1H, J=2.0 Hz), 7.64 (d, 1H, J=2.2 Hz), 5.23 (q, 1H, J=8.5).

Therapeutic Illustrations

The following non-limiting illustrative examples describe various neoplasia disorders or cancer diseases and therapeutic approaches that may be used in the present invention, and are for illustrative purposes only. Some Cox-2 selective inhibiting agents (or prodrugs thereof) that can be used in the below non-limiting illustrations include, but are not limited to, celecoxib, rofecoxib, valdecoxib, parecoxib, deracoxib, MK-663 and JTE-522, and some DNA topoisomerase I inhibiting agents that can be used with the below non-limiting illustrations include, for example, irinotecan, rubitecan, lurtotecan, exetecan mesylate, karenitecan, or silatecan.

Illustration 1

Lung Cancer

In many countries including Japan, Europe and America, the number of patients with lung cancer is fairly large and continues to increase year after year and is the most frequent cause of cancer death in both men and women. Although there are many potential causes for lung cancer, tobacco use, and particularly cigarette smoking, is the most important. Additionally, etiologic factors such as exposure to asbestos, especially in smokers, or radon are contributory factors. Also, occupational hazards such as exposure to uranium have been identified as an important factor. Finally, genetic factors have also been identified as another factor that increases the risk of cancer.

Lung cancers can be histologically classified into non-small cell lung cancers (e.g. squamous cell carcinoma (epidermoid), adenocarcinoma, large cell carcinoma (large cell anaplastic), etc.) and small cell lung cancer (oat cell). Non-small cell lung cancer (NSCLC) has different biological properties and responses to chemotherapeutics from those of small cell lung cancer (SCLC). Thus, chemotherapeutic formulas and radiation therapy are different between these two types of lung cancer.

Non-Small Cell Lung Cancer

Where the location of the non-small cell lung cancer tumor can be easily excised (stage I and II disease) surgery is the first line of therapy and offers a relatively good chance for a cure. However, in more advanced disease (stage IIa and greater), where the tumor has extended to tissue beyond the bronchopulmonary lymph nodes, surgery may not lead to complete excision of the tumor. In such cases, the patient's chance for a cure by surgery alone is greatly diminished. Where surgery will not provide complete removal of the NSCLC tumor, other types of therapies must be utilized.

Today radiation therapy is the standard treatment to control unresectable or inoperable NSCLC. Improved results have been seen when radiation therapy has been combined with chemotherapy, but gains have been modest and the search continues for improved methods of combining modalities.

Radiation therapy is based on the principle that high-dose radiation delivered to a target area will result in the death of reproductive cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. In one embodiment, a course of treatment for a patient undergoing radiation therapy for NSCLC will be a treatment with daily administration of a Cox-2 selective inhibiting agent and a DNA topoisomerase I inhibiting agents and a treatment schedule over a 5 to 6 week period with a total dose of 50 to 60 Gy administered to the patient in a single daily fraction of 1.8 to 2.0 Gy, 5 days a week. A Gy is an abbreviation for Gray and refers to 100 rad of dose.

However, as NSCLC is a systemic disease, and radiation therapy is a local modality, radiation therapy as a single line of therapy is unlikely to provide a cure for NSCLC, at least for those tumors that have metastasized distantly outside the zone of treatment. Thus, the use of radiation therapy with other modality regimens of the present invention have important potential beneficial effects for the treatment of NSCLC.

Generally, radiation therapy has been combined temporally with chemotherapy to improve the outcome of treatment. There are various terms to describe the temporal relationship of administering radiation therapy in combination with a Cox-2 selective inhibiting agent, a DNA topoisomerase I inhibiting agents and chemotherapy, and the following examples are some treatment regimens and are provided for illustration only and are not intended to limit the use of other combinations. “Sequential” therapy refers to the administration of chemotherapy and/or a Cox-2 selective inhibiting agent and/or a DNA topoisomerase I inhibiting agents and/or radiation therapy separately in time in order to allow the separate administration of chemotherapy and/or a Cox-2 selective inhibiting agent and/or a DNA topoisomerase I inhibiting agents and/or radiation therapy. “Concomitant” therapy refers to the administration of chemotherapy and/or a Cox-2 selective inhibiting agent and/or a DNA topoisomerase I inhibiting agents and/or radiation therapy on the same day. Finally, “alternating therapy refers to the administration of radiation therapy on the days in which chemotherapy and/or a Cox-2 selective inhibiting agent and/or a DNA topoisomerase I inhibiting agents would not have been administered if it was given alone.

Several chemotherapeutic agents have been shown to be efficacious against NSCLC. In one embodiment, chemotherapeutic agents that can be used in the methods, combinations and compositions of the present invention against NSCLC include etoposide, carboplatin, methotrexate, 5-Fluorouracil, epirubicin, doxorubicin, taxol, inhibitor of normal mitotic activity; and cyclophosphamide. In another embodiment, chemotherapeutic agents that may be used in the methods, combinations and compositions of the present invention active against NSCLC include cisplatin, ifosfamide, mitomycin C, epirubicin, vinblastine, and vindesine.

Other agents that are under investigation for use against NSCLC include: camptothecins, a topoisomerase 1 inhibitor; navelbine (vinorelbine), a microtubule assembly inhibitor; gemcitabine, a deoxycytidine analogue; fotemustine, a nitrosourea compound; and edatrexate, an antifol.

The overall and complete response rates for NSCLC has been shown to increase with use of combination chemotherapy as compared to single-agent treatment. Haskel C M: Chest. 99: 1325, 1991; Bakowski M T: Cancer Treat Rev 10: 159, 1983; Joss R A: Cancer Treat Rev 11:205, 1984.

In one embodiment, therapy for the treatment of NSCLC is a combination of neoplasia disorder effective amounts of a Cox-2 selective inhibiting agent and a DNA topoisomerase I inhibiting agents in combination with one or more of the following combinations of antineoplastic agents: 1) itosfamide, cisplatin, etoposide; 2) cyclophosphamide, doxorubicin, cisplatin; 3) isofamide, carboplatin, etoposide; 4) bleomycin, etoposide, cisplatin; 5) isofamide, mitomycin, cisplatin; 6) cisplatin, vinblastine; 7) cisplatin, vindesine; 8) mitomycin C, vinblastine, cisplatin; 9) mitomycin C, vindesine, cisplatin; 10) isofamide, etoposide; 11) etoposide, cisplatin; 12) isofamide, mitomycin C; 13) fluorouracil, cisplatin, vinblastine; 14) carboplatin, etoposide; or radiation therapy.

Small Cell Lung Cancer

Approximately 15 to 20 percent of all cases of lung cancer reported worldwide is small cell lung cancer (SCLC). Ihde D C: Cancer 54:2722, 1984. Currently, treatment of SCLC incorporates multi-modal therapy, including chemotherapy, radiation therapy and surgery. Response rates of localized or disseminated SCLC remain high to systemic chemotherapy, however, persistence of the primary tumor and persistence of the tumor in the associated lymph nodes has led to the integration of several therapeutic modalities in the treatment of SCLC.

In one embodiment, a therapy for the treatment of lung cancer is a combination of neoplasia disorder effective amounts of a Cox-2 selective inhibiting agent and a DNA topoisomerase I inhibiting agents, in combination with one or more of the following antineoplastic agents: vincristine, cisplatin, carboplatin, cyclophosphamide, epirubicin (high dose), etoposide (VP-16) I.V., etoposide (VP-16) oral, isofamide, teniposide (VM-26), and doxorubicin. Other single-agents chemotherapeutic agents that may be used in the methods, combinations and compositions of the present invention include BCNU (carmustine), vindesine, hexamethylmelamine (altretamine), methotrexate, nitrogen mustard, and CCNU (lomustine). Other chemotherapeutic agents under investigation that have shown activity against SCLC include iroplatin, gemcitabine, lonidamine, and taxol. Single-agent chemotherapeutic agents that have not yet shown activity against SCLC include mitoguazone, mitomycin C, aclarubicin, diaziquone, bisantrene, cytarabine, idarubicin, mitomxantrone, vinblastine, PCNU and esorubicin.

Another contemplated therapy for the treatment of SCLC is a combination of neoplasia disorder effective amounts a Cox-2 selective inhibiting agent and a DNA topoisomerase I inhibiting agents in combination with one or more of the following combinations of antineoplastic agents: 1) etoposide (VP-16), cisplatin; 2) cyclophosphamide, adriamycin [(doxorubicin), vincristine, etoposide (VP-16)]; 3) Cyclophosphamide, adriamycin(doxorubicin), vincristine; 4) Etoposide (VP-16), ifosfamide, cisplatin; 5) etoposide (VP-16), carboplatin; 6) cisplatin, vincristine (Oncovin), doxorubicin, etoposide.

Additionally, radiation therapy in conjunction with combinations of a Cox-2 selective inhibiting agent and a DNA topoisomerase I inhibiting agents and/or systemic chemotherapy is contemplated to be effective at increasing the response rate for SCLC patients. The typical dosage regimen for radiation therapy ranges from 40 to 55 Gy, in 15 to 30 fractions, 3 to 7 times week. The tissue volume to be irradiated is determined by several factors and generally the hilum and subcarnial nodes, and bilateral distal nodes up to the thoracic inlet are treated, as well as the primary tumor up to 1.5 to 2.0 cm of the margins.

Illustration 2

Colorectal Cancer

Survival from colorectal cancer depends on the stage and grade of the tumor, for example precursor adenomas to metastatic adenocarcinoma. Generally, colorectal cancer can be treated by surgically removing the tumor, but overall survival rates remain between 45 and 60 percent. Colonic excision morbidity rates are fairly low and is generally associated with the anastomosis and not the extent of the removal of the tumor and local tissue. In patients with a high risk of reoccurrence, however, chemotherapy has been incorporated into the treatment regimen in order to improve survival rates.

Tumor metastasis prior to surgery is generally believed to be the cause of surgical intervention failure and up to one year of chemotherapy is required to kill the non-excised tumor cells. As severe toxicity is associated with the chemotherapeutic agents, only patients at high risk of recurrence are placed on chemotherapy following surgery. Thus, the incorporation of a Cox-2 and a DNA topoisomerase I inhibiting agents into the management of colorectal cancer will play an important role in the treatment of colorectal cancer and lead to overall improved survival rates for patients diagnosed with colorectal cancer.

In one embodiment, a combination therapy for the treatment of colorectal cancer is surgery, followed by a regimen of one or more chemotherapeutic agents and a Cox-2 selective inhibiting agent and a DNA topoisomerase I inhibiting agents, cycled over a one year time period. In another embodiment, a combination therapy for the treatment of colorectal cancer is a regimen of a Cox-2 selective inhibiting agent and a DNA topoisomerase I inhibiting agents, followed by surgical removal of the tumor from the colon or rectum and then followed be a regimen of one or more chemotherapeutic agents and a Cox-2 selective inhibiting agent and a DNA topoisomerase I inhibiting agent, cycled over a one year time period. In still another embodiment, a therapy for the treatment of colon cancer is a combination of neoplasia disorder effective amounts of a Cox-2 selective inhibiting agent and a DNA topoisomerase I inhibiting agents.

In another embodiment, a therapy for the treatment of colon cancer is a combination of neoplasia disorder effective amounts of a Cox-2 selective inhibiting agent and a DNA topoisomerase I inhibiting agents in combination with fluorouracil and Levamisole. Typically, fluorouracil and Levamisole are used in combination.

In yet another embodiment, a therapy for the treatment of colon cancer is a combination of neoplasia disorder effective amounts of a Cox-2 selective inhibiting agent and a DNA topoisomerase I inhibiting agents in combination with fluorouracil and leucovorin. Typically, fluorouracil and leucovorin are used in combination.

Illustration 3

Breast Cancer

Today, among women in the United States, breast cancer remains the most frequent diagnosed cancer. One in 8 women in the United States are at risk of developing breast cancer in their lifetime. Age, family history, diet, and genetic factors have been identified as risk factors for breast cancer. Breast cancer is the second leading cause of death among women.

Different chemotherapeutic agents are known in art for treating breast cancer. Cytoxic agents used for treating breast cancer include doxorubicin, cyclophosphamide, methotrexate, 5-fluorouracil, mitomycin C, mitoxantrone, taxol, and epirubicin.

In the treatment of locally advanced noninflammatory breast cancer, a Cox-2 selective inhibiting agent and a DNA topoisomerase I inhibiting agents can be used to treat the disease in combination with surgery, radiation therapy and/or chemotherapy. Combinations of chemotherapeutic agents, radiation therapy and surgery that can be used in combination with the present invention include, but are not limited to the following combinations: 1) doxorubicin, vincristine, radical mastectomy; 2) doxorubicin, vincristine, radiation therapy; 3) cyclophosphamide, doxorubicin, 5-flourouracil, vincristine, prednisone, mastectomy; 4) cyclophosphamide, doxorubicin, 5-flourouracil, vincristine, prednisone, radiation therapy; 5) cyclophosphamide, doxorubicin, 5-flourouracil, premarin, tamoxifen, radiation therapy for pathologic complete response; 6) cyclophosphamide, doxorubicin, 5-flourouracil, premarin, tamoxifen, mastectomy, radiation therapy for pathologic partial response; 7) mastectomy, radiation therapy, levamisole; 8) mastectomy, radiation therapy; 9) mastectomy, vincristine, doxorubicin, cyclophosphamide, levamisole; 10) mastectomy, vincristine, doxorubicin, cyclophosphamide; 11) mastectomy, cyclophosphamide, doxorubicin, 5-fluorouracil, tamoxifen, halotestin, radiation therapy; 12) mastectomy, cyclophosphamide, doxorubicin, 5-fluorouracil, tamoxifen, halotestin.

In the treatment of locally advanced inflammatory breast cancer, a Cox-2 selective inhibiting agent and a DNA topoisomerase I inhibiting agents can be used to treat the disease in combination with surgery, radiation therapy or with chemotherapeutic agents. In one embodiment, combinations of chemotherapeutic agents, radiation therapy and surgery that can be used in combination with the present invention include, but or not limited to, the following combinations: 1) cyclophosphamide, doxorubicin, 5-fluorouracil, radiation therapy; 2) cyclophosphamide, doxorubicin, 5-fluorouracil, mastectomy, radiation therapy; 3) 5-fluorouracil, doxorubicin, clyclophosphamide, vincristine, prednisone, mastectomy, radiation therapy; 4) 5-fluorouracil, doxorubicin, clyclophosphamide, vincristine, mastectomy, radiation therapy; 5) cyclophosphamide, doxorubicin, 5-fluorouracil, vincristine, radiation therapy; 6) cyclophosphamide, doxorubicin, 5-fluorouracil, vincristine, mastectomy, radiation therapy; 7) doxorubicin, vincristine, methotrexate, radiation therapy, followed by vincristine, cyclophosphamide, 5-florouracil; 8) doxorubicin, vincristine, cyclophosphamide, methotrexate, 5-florouracil, radiation therapy, followed by vincristine, cyclophosphamide, 5-florouracil; 9) surgery, followed by cyclophosphamide, methotrexate, 5-fluorouracil, prednisone, tamoxifen, followed by radiation therapy, followed by cyclophosphamide, methotrexate, 5-fluorouracil, prednisone, tamoxifen, doxorubicin, vincristine, tamoxifen; 10) surgery, followed by cyclophosphamide, methotrexate, 5-fluorouracil, followed by radiation therapy, followed by cyclophosphamide, methotrexate, 5-fluorouracil, prednisone, tamoxifen, doxorubicin, vincristine, tamoxifen; 11) surgery, followed by cyclophosphamide, methotrexate, 5-fluorouracil, prednisone, tamoxifen, followed by radiation therapy, followed by cyclophosphamide, methotrexate, 5-fluorouracil, doxorubicin, vincristine, tamoxifen; 12) surgery, followed by cyclophosphamide, methotrexate, 5-fluorouracil, followed by radiation therapy, followed by cyclophosphamide, methotrexate, 5-fluorouracil, prednisone, tamoxifen, doxorubicin, vincristine; 13) surgery, followed by cyclophosphamide, methotrexate, 5-fluorouracil, prednisone, tamoxifen, followed by radiation therapy, followed by cyclophosphamide, methotrexate, 5-fluorouracil, prednisone, tamoxifen, doxorubicin, vincristine, tamoxifen; 14) surgery, followed by cyclophosphamide, methotrexate, 5-fluorouracil, followed by radiation therapy, followed by cyclophosphamide, methotrexate, 5-fluorouracil, prednisone, tamoxifen, doxorubicin, vincristine; 15) surgery, followed by cyclophosphamide, methotrexate, 5-fluorouracil, prednisone, tamoxifen, followed by radiation therapy, followed by cyclophosphamide, methotrexate, 5-fluorouracil, doxorubicin, vincristine; 16) 5-florouracil, doxorubicin, cyclophosphamide followed by mastectomy, followed by 5-florouracil, doxorubicin, cyclophosphamide, followed by radiation therapy.

In the treatment of metastatic breast cancer, a Cox-2 selective inhibiting agent and a DNA topoisomerase I inhibiting agents can be used to treat the disease in combination with surgery, radiation therapy and/or with chemotherapeutic agents. In one embodiment, combinations of chemotherapeutic agents that can be used in combination with a Cox-2 selective inhibiting agent and a DNA topoisomerase I inhibiting agents of the present invention, include, but are not limited to, the following combinations: 1) cyclophosphamide, methotrexate, 5-fluorouracil; 2) cyclophosphamide, adriamycin, 5-fluorouracil; 3) cyclophosphamide, methotrexate, 5-fluorouracil, vincristine, prednisone; 4) adriamycin, vincristine; 5) thiotepa, adriamycin, vinblastine; 6) mitomycin, vinblastine; 7) cisplatin, etoposide.

Illustration 4

Prostate Cancer

Prostate cancer is now the leading form of cancer among men and the second most frequent cause of death from cancer in men. It is estimated that more than 165,000 new cases of prostate cancer were diagnosed in 1993, and more than 35,000 men died from prostate cancer in that year. Additionally, the incidence of prostate cancer has increased by 50% since 1981, and mortality from this disease has continued to increase. Previously, most men died of other illnesses or diseases before dying from their prostate cancer. We now face increasing morbidity from prostate cancer as men live longer and the disease has the opportunity to progress.

Current therapies for prostate cancer focus exclusively upon reducing levels of dihydrotestosterone to decrease or prevent growth of prostate cancer. In addition to the use of digital rectal examination and transrectal ultrasonography, prostate-specific antigen (PSA) concentration is frequently used in the diagnosis of prostate cancer.

In one embodiment, a therapy for the treatment of prostate cancer is a combination of neoplasia disorder effective amounts of a Cox-2 selective inhibiting agent and a DNA topoisomerase I inhibiting agents.

U.S. Pat. No. 4,472,382 discloses treatment of benign prostatic hyperplasia (BPH) with an antiandrogen and certain peptides which act as LH-RH agonists.

U.S. Pat. No. 4,596,797 discloses aromatase inhibitors as a method of prophylaxis and/or treatment of prostatic hyperplasia.

U.S. Pat. No. 4,760,053 describes a treatment of certain cancers a LHRH agonist with an antiandrogen and/or an antiestrogen and/or at least one inhibitor of sex steroid biosynthesis.

U.S. Pat. No. 4,775,660 discloses a method of treating breast cancer with a combination therapy which may include surgical or chemical prevention of ovarian secretions and administering an antiandrogen and an antiestrogen.

U.S. Pat. No. 4,659,695 discloses a method of treatment of prostate cancer in susceptible male animals including humans whose testicular hormonal secretions are blocked by surgical or chemical means, e.g. by use of an LHRH agonist, which comprises administering an antiandrogen, e.g. flutamide, in association with at least one inhibitor of sex steroid biosynthesis, e.g. aminoglutethimide and/or ketoconazole.

Illustration 5

Bladder Cancer

The classification of bladder cancer is divided into three main classes: 1) superficial disease, 2) muscle-invasive disease, and 3) metastatic disease.

Currently, transurethral resection (TUR), or segmental resection, account for first line therapy of superficial bladder cancer, i.e., disease confined to the mucosa or the lamina propria. However, intravesical therapies are necessary, for example, for the treatment of high-grade tumors, carcinoma in situ, incomplete resections, recurrences, and multifocal papillary. Recurrence rates range from up to 30 to 80 percent, depending on stage of cancer.

Therapies that are currently used as intravesical therapies include chemotherapy, immuontherapy, bacille Calmette-Guerin (BCG) and photodynamic therapy. The main objective of intravesical therapy is twofold: to prevent recurrence in high-risk patients and to treat disease that cannot by resected. The use of intravesical therapies must be balanced with its potentially toxic side effects. Additionally, BCG requires an unimpaired immune system to induce an antitumor effect. Chemotherapeutic agents that are known to be of limited use against superficial bladder cancer include Cisplatin, actinomycin D, 5-fluorouracil, bleomycin, and cyclophosphamide methotrexate.

In the treatment of superficial bladder cancer, a Cox-2 selective inhibiting agent and a DNA topoisomerase I inhibiting agents can be used to treat the disease in combination with surgery (TUR), chemotherapy and/or intravesical therapies.

A therapy for the treatment of superficial bladder cancer is a combination of neoplasia disorder effective amounts of a Cox-2 selective inhibiting agent and a DNA topoisomerase I inhibiting agents in combination with: thiotepa (30 to 60 mg/day), mitomycin C (20 to 60 mg/day), and doxorubicin (20 to 80 mg/day).

In one embodiment, intravesicle immunotherapeutic agent that may be used in the methods, combinations and compositions of the present invention is BCG. A daily dose ranges from 60 to 120 mg, depending on the strain of the live attenuated tuberculosis organism used.

In another embodiment, a photodynamic therapeutic agent that may be used with the present invention is Photofrin I, a photosensitizing agent, administered intravenously. It is taken up by the low-density lipoprotein receptors of the tumor cells and is activated by exposure to visible light. Additionally, neomydium YAG laser activation generates large amounts of cytotoxic free radicals and singlet oxygen.

In the treatment of muscle-invasive bladder cancer, a Cox-2 selective inhibiting agent and a DNA topoisomerase I inhibiting agents can be used to treat the disease in combination with surgery (TUR), intravesical chemotherapy, radiation therapy, and/or radical cystectomy with pelvic lymph node dissection.

In one embodiment, radiation dose for the treatment of bladder cancer is between 5,000 to 7,000 cGY in fractions of 180 to 200 cGY to the tumor. Additionally, 3,500 to 4,700 cGY total dose is administered to the normal bladder and pelvic contents in a four-field technique. Radiation therapy should be considered only if the patient is not a surgical candidate, but may be considered as preoperative therapy.

In another embodiment, combination of surgery and chemotherapeutic agents that can be used in combination with a Cox-2 selective inhibiting agent and a DNA topoisomerase I inhibiting agents of the present invention is cystectomy in conjunction with five cycles of cisplatin (70 to 100 mg/m(square)); doxorubicin (50 to 60 mg/m(square); and cyclophosphamide (500 to 600 mg/m(square).

In one embodiment, a therapy for the treatment of superficial bladder cancer is a combination of neoplasia disorder effective amounts of a Cox-2 selective inhibiting agent and a DNA topoisomerase I inhibiting agents.

In another embodiment, a combination for the treatment of superficial bladder cancer is a combination of neoplasia disorder effective amounts of a Cox-2 selective inhibiting agent and a DNA topoisomerase I inhibiting agents in combination with one or more of the following combinations of antineoplastic agents: 1) cisplatin, doxorubicin, cyclophosphamide; and 2) cisplatin, 5-fluorouracil. A combination of chemotherapeutic agents that can be used in combination with radiation therapy and a Cox-2 selective inhibiting agent and a DNA topoisomerase inhibitor is a combination of cisplatin, methotrexate, vinblastine.

Currently no curative therapy exists for metastatic bladder cancer. The present invention contemplates an effective treatment of bladder cancer leading to improved tumor inhibition or regression, as compared to current therapies. In the treatment of metastatic bladder cancer, a Cox-2 selective inhibiting agent and a DNA topoisomerase I inhibiting agents can be used to treat the disease in combination with surgery, radiation therapy and/or with chemotherapeutic agents.

In one embodiment, a therapy for the treatment of metastatic bladder cancer is a combination of neoplasia disorder effective amounts of a Cox-2 selective inhibiting agent and a DNA topoisomerase I inhibiting agents. In another embodiment, therapy for the treatment of metastatic bladder cancer is a combination of neoplasia disorder effective amounts of a Cox-2 selective inhibiting agent and a DNA topoisomerase I inhibiting agents in combination with one or more of the following combinations of antineoplastic agents: 1) cisplatin and methotrexate; 2) doxorubicin, vinblastine, cyclophosphamide, and 5-fluorouracil; 3) vinblastine, doxorubicin, cisplatin, methotrexate; 4) vinblastine, cisplatin, methotrexate; 5) cyclophosphamide, doxorubicin, cisplatin; 6) 5-fluorouracil, cisplatin.

Illustration 6

Pancreas Cancer

Approximately 2% of new cancer cases diagnoses in the United States are pancreatic cancer. Pancreatic cancer is generally classified into two clinical types: 1) adenocarcinoma (metastatic and non-metastatic), and 2) cystic neoplasms (serous cystadenomas, mucinous cystic neoplasms, papillary cystic neoplasms, acinar cell systadenocarcinoma, cystic choriocarcinoma, cystic teratomas, angiomatous neoplasms).

In one embodiment, a therapy for the treatment of non-metastatic adenocarcinoma that may be used in the methods, combinations and compositions of the present invention include the use of a Cox-2 selective inhibiting agent and a DNA topoisomerase I inhibiting agents along with preoperative biliary tract decompression (patients presenting with obstructive jaundice); surgical resection, including standard resection, extended or radial resection and distal pancreatectomy (tumors of body and tail); adjuvant radiation; and/or chemotherapy.

In one embodiment for the treatment of metastatic adenocarcinoma, a therapy consists of a Cox-2 selective inhibiting agent and a DNA topoisomerase I inhibiting agents of the present invention in combination with continuous treatment of 5-fluorouracil, followed by weekly cisplatin therapy.

In another embodiment, a combination therapy for the treatment of cystic neoplasms is the use of a Cox-2 selective inhibiting agent and a DNA topoisomerase I inhibiting agents along with resection.

Illustration 7

Ovary Cancer

Celomic epithelial carcinoma accounts for approximately 90% of ovarian cancer cases. In one embodiment, a therapy for the treatment of ovary cancer is a combination of neoplasia disorder effective amounts of a Cox-2 selective inhibiting agent and a DNA topoisomerase I inhibiting agents.

Single agents that can be used in combination with a Cox-2 selective inhibiting agent and a DNA topoisomerase I inhibiting agents include, but are not limited to: alkylating agents, ifosfamide, cisplatin, carboplatin, taxol, doxorubicin, 5-fluorouracil, methotrexate, mitomycin, hexamethylmelamine, progestins, antiestrogens, prednimustine, dihydroxybusulfan, galactitol, interferon alpha, and interferon gamma.

In another embodiment, combinations for the treatment of celomic epithelial carcinoma is a combination of neoplasia disorder effective amounts of a Cox-2 selective inhibiting agent and a DNA topoisomerase I inhibiting agents in combination with one or more of the following combinations of antineoplastic agents: 1) cisplatin, doxorubicin, cyclophosphamide; 2) hexamthylmelamine, cyclophosphamide, doxorubicin, cisplatin; 3) cyclophosphamide, hexamehtylmelamine, 5-fluorouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethlmelamine, doxorubicin, cisplatin; 12) carboplatin, cyclophosphamide; 13) cisplatin, cyclophosphamide.

Germ cell ovarian cancer accounts for approximately 5% of ovarian cancer cases. Germ cell ovarian carcinomas are classified into two main groups: 1) dysgerminoma, and nondysgerminoma. Nondysgerminoma is further classified into teratoma, endodermal sinus tumor, embryonal carcinoma, chloricarcinoma, polyembryoma, and mixed cell tumors.

In one embodiment, a therapy for the treatment of germ cell carcinoma is a combination of neoplasia disorder effective amounts of a Cox-2 selective inhibiting agent and a DNA topoisomerase I inhibiting agents.

In another embodiment, a therapy for the treatment of germ cell carcinoma is a combination of neoplasia disorder effective amounts of a Cox-2 selective inhibiting agent and a DNA topoisomerase I inhibiting agents in combination with one or more of the following combinations of antineoplastic agents: 1) vincristine, actinomycin D, cyclophosphamide; 2) bleomycin, etoposide, cisplatin; 3) vinblastine, bleomycin, cisplatin.

Cancer of the fallopian tube is the least common type of ovarian cancer, accounting for approximately 400 new cancer cases per year in the United States. Papillary serous adenocarcinoma accounts for approximately 90% of all malignancies of the ovarian tube.

In one embodiment, a therapy for the treatment of fallopian tube cancer is a combination of neoplasia disorder effective amounts of a Cox-2 selective inhibiting agent and a DNA topoisomerase I inhibiting agents.

In another embodiment, a therapy for the treatment of fallopian tube cancer is a combination of neoplasia disorder effective amounts of a Cox-2 selective inhibiting agent and a DNA topoisomerase I inhibiting agents in combination with on or more of the following of antineoplastic agents: alkylating agents, ifosfamide, cisplatin, carboplatin, taxol, doxorubicin, 5-fluorouracil, methotrexate, mitomycin, hexamethylmelamine, progestins, antiestrogens, prednimustine, dihydroxybusulfan, galactitol, interferon alpha, and interferon gamma.

In still another embodiment, therapy for the treatment of fallopian tube cancer is a combination of neoplasia disorder effective amounts of a Cox-2 selective inhibiting agent and a DNA topoisomerase I inhibiting agents in combination with one or more of the following combinations of antineoplastic agents: 1) cisplatin, doxorubicin, cyclophosphamide; 2) hexamthylmelamine, cyclophosphamide, doxorubicin, cisplatin; 3) cyclophosphamide, hexamehtylmelamine, 5-fluorouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethimelamine, doxorubicin, cisplatin; 12) carboplatin, cyclophosphamide; 13) cisplatin, cyclophosphamide.

Illustration 8

Central Nervous System Cancers

Central nervous system cancer accounts for approximately 2% of new cancer cases in the United States. Common intracranial neoplasms include glioma, meninigioma, neurinoma, and adenoma.

In one embodiment, a therapy for the treatment of central nervous system cancers is a combination of neoplasia disorder effective amounts of a Cox-2 selective inhibiting agent and a DNA topoisomerase I inhibiting agent.

In another embodiment, a therapy for the treatment of malignant glioma is a combination of neoplasia disorder effective amounts of a Cox-2 selective inhibiting agent and a DNA topoisomerase I inhibiting agents in combination with one or more of the following combinations of therapies and antineoplastic agents: 1) radiation therapy, BCNU (carmustine); 2) radiation therapy, methyl CCNU (lomustine); 3) radiation therapy, medol; 4) radiation therapy, procarbazine; 5) radiation therapy, BCNU, medrol; 6) hyperfraction radiation therapy, BCNU; 7) radiation therapy, misonidazole, BCNU; 8) radiation therapy, streptozotocin; 9) radiation therapy, BCNU, procarbazine; 10) radiation therapy, BCNU, hydroxyurea, procarbazine, VM-26; 11) radiation therapy, BNCU, 5-flourouacil; 12) radiation therapy, Methyl CCNU, dacarbazine; 13) radiation therapy, misonidazole, BCNU; 14) diaziquone; 15) radiation therapy, PCNU; 16) procarbazine (matulane), CCNU, vincristine. A dose of radiation therapy is about 5,500 to about 6,000 cGY. Radiosensitizers include misonidazole, intra-arterial Budr and intravenous iododeoxyuridine (IUdR). It is also contemplated that radiosurgery may be used in combinations with a Cox-2 selective inhibiting agent and a DNA topoisomerase I inhibiting agent.

Illustration 9

Table Nos. 22 and 23 provide additional non-limiting illustrative examples of combination therapies that can be used in the methods, combinations and compositions of the present invention. In each combination identified in Table Nos. 22 and 23, the individual combination is used in combination with an aromatase inhibiting agent. Exemplary aromatase inhibiting agents that can be used in the below non-limiting illustrative examples include anastrozole, atamestane, exemestane, fadrozole, finrozol, formestane, letrozole, minamestane, MR-20492, Testolactone, YM-511, and vorozole. Other examples of aromatase inhibiting agents that can be used in the combinations of the below examples are provided in Table No. 3, above. Additionally, non-limiting illustrative examples of combinations of Cox-2 selective inhibiting agents and aromatase inhibiting agents are provided in Table No. 24 below. Table No. 22 provides non-limiting illustrative examples of a Cox-2 selective inhibiting agent in combination with a single antineoplastic agent in the treatment of an illustrative neoplasia disorder. Table No. 23 provides non-limiting illustrative examples of a Cox-2 selective inhibiting agent in combination with multiple antineoplastic agents in the treatment of an illustrative neoplasia disorder.

TABLE No. 22
A Cox-2 Inhibiting Agent in Combination with a Single
Antineoplastic Agent.

| Cox-2 | Antineoplastic | |
|---|---|---|
| Inhibitor | Agents | Indication |
| Celecoxib | Anastrozole | Breast |
| Celecoxib | Capecitabine | Breast |
| Celecoxib | Docetaxel | Breast |
| Celecoxib | Gemcitabine | Breast, Pancreas |
| Celecoxib | Letrozole | Breast |
| Celecoxib | Megestrol | Breast |
| Celecoxib | Paclitaxel | Breast |
| Celecoxib | Tamoxifen | Breast |
| Celecoxib | Toremifene | Breast |
| Celecoxib | Vinorelbine | Breast, Lung |
| Celecoxib | Topotecan | Lung |
| Celecoxib | Etoposide | Lung |
| Celecoxib | Fluorouracil | Colon |
| Celecoxib | Irinotecan (CPT-11) | Colon, Bladder |
| Celecoxib | Retinoids | Colon |
| Celecoxib | DFMO | Colon |
| Celecoxib | Ursodeoxycholic acid | Colon |
| Celecoxib | Calcium carbonate | Colon |
| Celecoxib | Selenium | Colon |
| Celecoxib | Sulindac sulfone | Colon |
| Celecoxib | Carboplatin | Brain |
| Celecoxib | Goserelin Acetate | Prostate |
| Celecoxib | Cisplatin | Lung |
| Celecoxib | Ketoconazole | Prostate |
| Rofecoxib | Anastrozole | Breast |
| Rofecoxib | Capecitabine | Breast |
| Rofecoxib | Docetaxel | Breast |
| Rofecoxib | Gemcitabine | Breast, Pancreas |
| Rofecoxib | Letrozole | Breast |
| Rofecoxib | Megestrol | Breast |
| Rofecoxib | Paclitaxel | Breast |
| Rofecoxib | Tamoxifen | Breast |
| Rofecoxib | Toremifene | Breast |
| Rofecoxib | Vinorelbine | Breast, Lung |
| Rofecoxib | Topotecan | Lung |
| Rofecoxib | Etoposide | Lung |
| Rofecoxib | Fluorouracil | Colon |
| Rofecoxib | Irinotecan (CPT-11) | Colon, Bladder |
| Rofecoxib | Retinoids | Colon |
| Rofecoxib | DFMO | Colon |
| Rofecoxib | Ursodeoxycholic acid | Colon |
| Rofecoxib | Calcium carbonate | Colon |
| Rofecoxib | Selenium | Colon |
| Rofecoxib | Sulindac sulfone | Colon |
| Rofecoxib | Carboplatin | Brain |
| Rofecoxib | Goserelin Acetate | Prostate |
| Rofecoxib | Cisplatin | Lung |
| Rofecoxib | Ketoconazole | Prostate |
| Valdecoxib | Anastrozole | Breast |
| Valdecoxib | Capecitabine | Breast |
| Valdecoxib | Docetaxel | Breast |
| Valdecoxib | Gemcitabine | Breast, Pancreas |
| Valdecoxib | Letrozole | Breast |
| Valdecoxib | Megestrol | Breast |
| Valdecoxib | Paclitaxel | Breast |
| Valdecoxib | Tamoxifen | Breast |
| Valdecoxib | Toremifene | Breast |
| Valdecoxib | Vinorelbine | Breast, Lung |
| Valdecoxib | Topotecan | Lung |
| Valdecoxib | Etoposide | Lung |
| Valdecoxib | Fluorouracil | Colon |
| Valdecoxib | Irinotecan (CPT-11) | Colon, Bladder |
| Valdecoxib | Retinoids | Colon |
| Valdecoxib | DFMO | Colon |
| Valdecoxib | Ursodeoxycholic acid | Colon |
| Valdecoxib | Calcium carbonate | Colon |
| Valdecoxib | Selenium | Colon |
| Valdecoxib | Sulindac sulfone | Colon |
| Valdecoxib | Carboplatin | Brain |
| Valdecoxib | Goserelin Acetate | Prostate |
| Valdecoxib | Cisplatin | Lung |
| Valdecoxib | Ketoconazole | Prostate |
| Deracoxib | Anastrozole | Breast |
| Deracoxib | Capecitabine | Breast |
| Deracoxib | Docetaxel | Breast |
| Deracoxib | Gemcitabine | Breast, Pancreas |
| Deracoxib | Letrozole | Breast |
| Deracoxib | Megestrol | Breast |
| Deracoxib | Paclitaxel | Breast |
| Deracoxib | Tamoxifen | Breast |
| Deracoxib | Toremifene | Breast |
| Deracoxib | Vinorelbine | Breast, Lung |
| Deracoxib | Topotecan | Lung |
| Deracoxib | Etoposide | Lung |
| Deracoxib | Fluorouracil | Colon |
| Deracoxib | Irinotecan (CPT-11) | Colon, Bladder |
| Deracoxib | Retinoids | Colon |
| Deracoxib | DFMO | Colon |
| Deracoxib | Ursodeoxycholic acid | Colon |
| Deracoxib | Calcium carbonate | Colon |
| Deracoxib | Selenium | Colon |
| Deracoxib | Sulindac sulfone | Colon |
| Deracoxib | Carboplatin | Brain |
| Deracoxib | Goserelin Acetate | Prostate |
| Deracoxib | Cisplatin | Lung |
| Deracoxib | Ketoconazole | Prostate |
| JTE-522 | Anastrozole | Breast |
| JTE-522 | Capecitabine | Breast |
| JTE-522 | Docetaxel | Breast |
| JTE-522 | Gemcitabine | Breast, Pancreas |
| JTE-522 | Letrozole | Breast |
| JTE-522 | Megestrol | Breast |
| JTE-522 | Paclitaxel | Breast |
| JTE-522 | Tamoxifen | Breast |
| JTE-522 | Toremifene | Breast |
| JTE-522 | Vinorelbine | Breast, Lung |
| JTE-522 | Topotecan | Lung |
| JTE-522 | Etoposide | Lung |
| JTE-522 | Fluorouracil | Colon |
| JTE-522 | Irinotecan (CPT-11) | Colon, Bladder |
| JTE-522 | Retinoids | Colon |
| JTE-522 | DFMO | Colon |
| JTE-522 | Ursodeoxycholic acid | Colon |
| JTE-522 | Calcium carbonate | Colon |
| JTE-522 | Selenium | Colon |
| JTE-522 | Sulindac sulfone | Colon |
| JTE-522 | Carboplatin | Brain |
| JTE-522 | Goserelin Acetate | Prostate |
| JTE-522 | Cisplatin | Lung |
| JTE-522 | Ketoconazole | Prostate |
| MK-663 | Anastrozole | Breast |
| MK-663 | Capecitabine | Breast |
| MK-663 | Docetaxel | Breast |
| MK-663 | Gemcitabine | Breast, Pancreas |
| MK-663 | Letrozole | Breast |
| MK-663 | Megestrol | Breast |
| MK-663 | Paclitaxel | Breast |
| MK-663 | Tamoxifen | Breast |
| MK-663 | Toremifene | Breast |
| MK-663 | Vinorelbine | Breast, Lung |
| MK-663 | Topotecan | Lung |
| MK-663 | Etoposide | Lung |
| MK-663 | Fluorouracil | Colon |
| MK-663 | Irinotecan (CPT-11) | Colon, Bladder |
| MK-663 | Retinoids | Colon |
| MK-663 | DFMO | Colon |
| MK-663 | Ursodeoxycholic acid | Colon |
| MK-663 | Calcium carbonate | Colon |
| MK-663 | Selenium | Colon |
| MK-663 | Sulindac sulfone | Colon |
| MK-663 | Carboplatin | Brain |
| MK-663 | Goserelin Acetate | Prostate |
| MK-663 | Cisplatin | Lung |
| MK-663 | Ketoconazole | Prostate |

TABLE No. 23
A Cox-2 Inhibiting Agent in Combination with Multiple
Antineoplastic Agents.

| Cox-2 | Antineoplastic | |
|---|---|---|
| Inhibitor | Agents | Indication |
| Celecoxib | Doxorubicin and | Breast |
| | Cyclophosphamide | |
| Celecoxib | Cyclophosphamide, | Breast |
| | Doxorubicin, and | |
| | Fluorouracil | |
| Celecoxib | Cyclophosphamide, | Breast |
| | Fluorouracil and | |
| | Mitoxantrone | |
| Celecoxib | Mitoxantrone, | Breast |
| | Fluorouracil and | |
| | Leucovorin | |
| Celecoxib | Vinblastine, Doxorubicin, | Breast |
| | Thiotepa, and | |
| | Fluoxymestrone | |
| Celecoxib | Cyclophosphamide, | Breast |
| | Methotrexate, | |
| | Fluorouracil | |
| Celecoxib | Doxorubicin, | Breast |
| | Cyclophosphamide, | |
| | Methotrexate, | |
| | Fluorouracil | |
| Celecoxib | Vinblastine, Doxorubicin, | Breast |
| | Thiotepa, | |
| | Fluoxymesterone | |
| Celecoxib | Fluorouracil, Levamisole | Colon |
| Celecoxib | Leucovorin, Fluorouracil | Colon |
| Celecoxib | Cyclophosphamide, | Lung |
| | Doxorubicin, Etoposide | |
| Celecoxib | Cyclophosphamide, | Lung |
| | Doxorubicin, Vincristine | |
| Celecoxib | Etoposide, Carboplatin | Lung |
| Celecoxib | Etoposide, Cisplatin | Lung |
| Celecoxib | Paclitaxel, Carboplatin | Lung |
| Celecoxib | Gemcitabine, Cisplatin | Lung |
| Celecoxib | Paclitaxel, Cisplatin | Lung |
| Rofecoxib | Doxorubicin and | Breast |
| | Cyclophosphamide | |
| Rofecoxib | Cyclophosphamide, | Breast |
| | Doxorubicin, and | |
| | Fluorouracil | |
| Rofecoxib | Cyclophosphamide, | Breast |
| | Fluorouracil and | |
| | Mitoxantrone | |
| Rofecoxib | Mitoxantrone, | Breast |
| | Flourouracil and | |
| | Leucovorin | |
| Rofecoxib | Vinblastine, Doxorubicin, | Breast |
| | Thiotepa, and | |
| | Fluoxymestrone | |
| Rofecoxib | Cyclophosphamide, | Breast |
| | Methotrexate, | |
| | Fluorouracil | |
| Rofecoxib | Doxorubicin, | Breast |
| | Cyclophosphamide, | |
| | Methotrexate, | |
| | Fluorouracil | |
| Rofecoxib | Vinblastine, Doxorubicin, | Breast |
| | Thiotepa, | |
| | Fluoxymesterone | |
| Rofecoxib | Fluorouracil, Levamisole | Colon |
| Rofecoxib | Leucovorin, Fluorouracil | Colon |
| Rofecoxib | Cyclophosphamide, | Lung |
| | Doxorubicin, Etoposide | |
| Rofecoxib | Cyclophosphamide, | Lung |
| | Doxorubicin, Vincristine | |
| Rofecoxib | Etoposide, Carboplatin | Lung |
| Rofecoxib | Etoposide, Cisplatin | Lung |
| Rofecoxib | Paclitaxel, Carboplatin | Lung |
| Rofecoxib | Gemcitabine, Cisplatin | Lung |
| Rofecoxib | Paclitaxel, Cisplatin | Lung |
| Valdecoxib | Doxorubicin and | Breast |
| | Cyclophosphamide | |
| Valdecoxib | Cyclophosphamide, | Breast |
| | Doxorubicin, and | |
| | Fluorouracil | |
| Valdecoxib | Cyclophosphamide, | Breast |
| | Fluorouracil and | |
| | Mitoxantrone | |
| Valdecoxib | Mitoxantrone, | Breast |
| | Fluorouracil and | |
| | Leucovorin | |
| Valdecoxib | Vinblastine, Doxorubicin, | Breast |
| | Thiotepa, and | |
| | Fluoxymestrone | |
| Valdecoxib | Cyclophosphamide, | Breast |
| | Methotrexate, | |
| | Fluorouracil | |
| Valdecoxib | Doxorubicin, | Breast |
| | Cyclophosphamide, | |
| | Methotrexate, | |
| | Fluorouracil | |
| Valdecoxib | Vinblastine, Doxorubicin, | Breast |
| | Thiotepa, | |
| | Fluoxymesterone | |
| Valdecoxib | Fluorouracil, Levamisole | Colon |
| Valdecoxib | Leucovorin, Fluorouracil | Colon |
| Valdecoxib | Cyclophosphamide, | Lung |
| | Doxorubicin, Etoposide | |
| Valdecoxib | Cyclophosphamide, | Lung |
| | Doxorubicin, Vincristine | |
| Valdecoxib | Etoposide, Carboplatin | Lung |
| Valdecoxib | Etoposide, Cisplatin | Lung |
| Valdecoxib | Paclitaxel, Carboplatin | Lung |
| Valdecoxib | Gemcitabine, Cisplatin | Lung |
| Valdecoxib | Paclitaxel, Cisplatin | Lung |
| Deracoxib | Doxorubicin and | Breast |
| | Cyclophosphamide | |
| Deracoxib | Cyclophosphamide, | Breast |
| | Doxorubicin, and | |
| | Fluorouracil | |
| Deracoxib | Cyclophosphamide, | Breast |
| | Fluorouracil and | |
| | Mitoxantrone | |
| Deracoxib | Mitoxantrone, | Breast |
| | Fluorouracil and | |
| | Leucovorin | |
| Deracoxib | Vinblastine, Doxorubicin, | Breast |
| | Thiotepa, and | |
| | Fluoxymestrone | |
| Deracoxib | Cyclophosphamide, | Breast |
| | Methotrexate, | |
| | Fluorouracil | |
| Deracoxib | Doxorubicin, | Breast |
| | Cyclophosphamide, | |
| | Methotrexate, | |
| | Fluorouracil | |
| Deracoxib | Vinblastine, Doxorubicin, | Breast |
| | Thiotepa, | |
| | Fluoxymesterone | |
| Deracoxib | Fluorouracil, Levamisole | Colon |
| Deracoxib | Leucovorin, Fluorouracil | Colon |
| Deracoxib | Cyclophosphamide, | Lung |
| | Doxorubicin, Etoposide | |
| Deracoxib | Cyclophosphamide, | Lung |
| | Doxorubicin, Vincristine | |
| Deracoxib | Etoposide, Carboplatin | Lung |
| Deracoxib | Etoposide, Cisplatin | Lung |
| Deracoxib | Paclitaxel, Carboplatin | Lung |
| Deracoxib | Gemcitabine, Cisplatin | Lung |
| Deracoxib | Paclitaxel, Cisplatin | Lung |
| JTE-522 | Doxorubicin and | Breast |
| | Cyclophosphamide | |
| JTE-522 | Cyclophosphamide, | Breast |
| | Doxorubicin, and | |
| | Fluorouracil | |
| JTE-522 | Cyclophosphamide, | Breast |
| | Fluorouracil and | |
| | Mitoxantrone | |
| JTE-522 | Mitoxantrone, | Breast |
| | Flourouracil and | |
| | Leucovorin | |
| JTE-522 | Vinblastine, Doxorubicin, | Breast |
| | Thiotepa, and | |
| | Fluoxymestrone | |
| JTE-522 | Cyclophosphamide, | Breast |
| | Methotrexate, | |
| | Fluorouracil | |
| JTE-522 | Doxorubicin, | Breast |
| | Cyclophosphamide, | |
| | Methotrexate, | |
| | Fluorouracil | |
| JTE-522 | Vinblastine, Doxorubicin, | Breast |
| | Thiotepa, | |
| | Fluoxymesterone | |
| JTE-522 | Fluorouracil, Levamisole | Colon |
| JTE-522 | Leucovorin, Fluorouracil | Colon |
| JTE-522 | Cyclophosphamide, | Lung |
| | Doxorubicin, Etoposide | |
| JTE-522 | Cyclophosphamide, | Lung |
| | Doxorubicin, Vincristine | |
| JTE-522 | Etoposide, Carboplatin | Lung |
| JTE-522 | Etoposide, Cisplatin | Lung |
| JTE-522 | Paclitaxel, Carboplatin | Lung |
| JTE-522 | Gemcitabine, Cisplatin | Lung |
| JTE-522 | Paclitaxel, Cisplatin | Lung |
| MK-663 | Doxorubicin and | Breast |
| | Cyclophosphamide | |
| MK-663 | Cyclophosphamide, | Breast |
| | Doxorubicin, and | |
| | Fluorouracil | |
| MK-663 | Cyclophosphamide, | Breast |
| | Fluorouracil and | |
| | Mitoxantrone | |
| MK-663 | Mitoxantrone, | Breast |
| | Fluorouracil and | |
| | Leucovorin | |
| MK-663 | Vinblastine, Doxorubicin, | Breast |
| | Thiotepa, and | |
| | Fluoxymestrone | |
| MK-663 | Cyclophosphamide, | Breast |
| | Methotrexate, | |
| | Fluorouracil | |
| MK-663 | Doxorubicin, | Breast |
| | Cyclophosphamide, | |
| | Methotrexate, | |
| | Fluorouracil | |
| MK-663 | Vinblastine, Doxorubicin, | Breast |
| | Thiotepa, | |
| | Fluoxymesterone | |
| MK-663 | Fluorouracil, Levamisole | Colon |
| MK-663 | Leucovorin, Fluorouracil | Colon |
| MK-663 | Cyclophosphamide, | Lung |
| | Doxorubicin, Etoposide | |
| MK-663 | Cyclophosphamide, | Lung |
| | Doxorubicin, Vincristine | |
| MK-663 | Etoposide, Carboplatin | Lung |
| MK-663 | Etoposide, Cisplatin | Lung |
| MK-663 | Paclitaxel, Carboplatin | Lung |
| MK-663 | Gemcitabine, Cisplatin | Lung |
| MK-663 | Paclitaxel, Cisplatin | Lung |
| MK-663 | Doxorubicin and | Breast |
| | Cyclophosphamide | |
| MK-663 | Cyclophosphamide, | Breast |
| | Doxorubicin, and | |
| | Fluorouracil | |
| MK-663 | Cyclophosphamide, | Breast |
| | Fluorouracil and | |
| | Mitoxantrone | |
| MK-663 | Mitoxantrone, | Breast |
| | Fluorouracil and | |
| | Leucovorin | |
| MK-663 | Vinblastine, Doxorubicin, | Breast |
| | Thiotepa, and | |
| | Fluoxymestrone | |
| MK-663 | Cyclophosphamide, | Breast |
| | Methotrexate, | |
| | Fluorouracil | |
| MK-663 | Doxorubicin, | Breast |
| | Cyclophosphamide, | |
| | Methotrexate, | |
| | Fluorouracil | |
| MK-663 | Vinblastine, Doxorubicin, | Breast |
| | Thiotepa, | |
| | Fluoxymesterone | |
| MK-663 | Fluorouracil, Levamisole | Colon |
| MK-663 | Leucovorin, Fluorouracil | Colon |
| MK-663 | Cyclophosphamide, | Lung |
| | Doxorubicin, Etoposide | |
| MK-663 | Cyclophosphamide, | Lung |
| | Doxorubicin, Vincristine | |
| MK-663 | Etoposide, Carboplatin | Lung |
| MK-663 | Etoposide, Cisplatin | Lung |
| MK-663 | Paclitaxel, Carboplatin | Lung |
| MK-663 | Gemcitabine, Cisplatin | Lung |
| MK-663 | Paclitaxel, Cisplatin | Lung |

Illustration 10

Table No. 24 illustrates examples of some combinations of the present invention where the combination comprises a Cox-2 selective inhibiting agent and a DNA topoisomerase I inhibiting agent.

TABLE No. 24
Combinations of Cox-2 selective inhibiting agents and
DNA topoisomerase I inhibiting agents

| Cox-2 selective | |
|---|---|
| inhibiting agent | DNA topoisomerase I inhibiting agent |
| Celecoxib | irinotecan |
| Rofecoxib | irinotecan |
| Valdecoxib | irinotecan |
| Deracoxib | irinotecan |
| JTE-522 | irinotecan |
| MK-663 | irinotecan |
| Celecoxib | camptothecin |
| Rofecoxib | camptothecin |
| Valdecoxib | camptothecin |
| Deracoxib | camptothecin |
| JTE-522 | camptothecin |
| MK-663 | camptothecin |
| Celecoxib | lurtotecan |
| Rofecoxib | lurtotecan |
| Valdecoxib | lurtotecan |
| Deracoxib | lurtotecan |
| JTE-522 | lurtotecan |
| MK-663 | lurtotecan |
| Celecoxib | homosilatecans |
| Rofecoxib | homosilatecans |
| Valdecoxib | homosilatecans |
| Deracoxib | homosilatecans |
| JTE-522 | homosilatecans |
| MK-663 | homosilatecans |
| Celecoxib | 9-amino camptothecin |
| Rofecoxib | 9-amino camptothecin |
| Valdecoxib | 9-amino camptothecin |
| Deracoxib | 9-amino camptothecin |
| JTE-522 | 9-amino camptothecin |
| MK-663 | 9-amino camptothecin |
| Celecoxib | 9-nitrocamptothecin |
| Rofecoxib | 9-nitrocamptothecin |
| Valdecoxib | 9-nitrocamptothecin |
| Deracoxib | 9-nitrocamptothecin |
| JTE-522 | 9-nitrocamptothecin |
| MK-663 | 9-nitrocamptothecin |
| Celecoxib | 4-Acridinecarboxamide, N-[2-(dimethylamino)ethyl]- |
| Rofecoxib | 4-Acridinecarboxamide, N-[2-(dimethylamino)ethyl]- |
| Valdecoxib | 4-Acridinecarboxamide, N-[2-(dimethylamino)ethyl]- |
| Deracoxib | 4-Acridinecarboxamide, N-[2-(dimethylamino)ethyl]- |
| JTE-522 | 4-Acridinecarboxamide, N-[2-(dimethylamino)ethyl]- |
| MK-663 | 4-Acridinecarboxamide, N-[2-(dimethylamino)ethyl]- |
| Celecoxib | 4-Acridinecarboxamide, N-[2-(dimethylamino)ethyl]-, |
| | dihydrochloride |
| Rofecoxib | 4-Acridinecarboxamide, N-[2-(dimethylamino)ethyl]-, |
| | dihydrochloride |
| Valdecoxib | 4-Acridinecarboxamide, N-[2-(dimethylamino)ethyl]-, |
| | dihydrochloride |
| Deracoxib | 4-Acridinecarboxamide, N-[2-(dimethylamino)ethyl]-, |
| | dihydrochloride |
| JTE-522 | 4-Acridinecarboxamide, N-[2-(dimethylamino)ethyl]-, |
| | dihydrochloride |
| MK-663 | 4-Acridinecarboxamide, N-[2-(dimethylamino)ethyl]-, |
| | dihydrochloride |
| Celecoxib | topotecan |
| Rofecoxib | topotecan |
| Valdecoxib | topotecan |
| Deracoxib | topotecan |
| JTE-522 | topotecan |
| MK-663 | topotecan |
| Celecoxib | topotecan hydrochloride |
| Rofecoxib | topotecan hydrochloride |
| Valdecoxib | topotecan hydrochloride |
| Deracoxib | topotecan hydrochloride |
| JTE-522 | topotecan hydrochloride |
| MK-663 | topotecan hydrochloride |

Evaluation of Cox-1 and Cox-2 Activity In Vitro

The Cox-2 selective inhibiting agents of this invention exhibit inhibition in vitro of Cox-2. The Cox-2 inhibition activity of the compounds illustrated in the Examples above were determined by the following methods. The Cox-2 inhibition activity of the other Cox-2 inhibitors of the present invention may also be determined by the following methods.

a. Preparation of Recombinant COX Baculoviruses

Recombinant Cox-1 and Cox-2 were prepared as described by Gierse et al, [J. Biochem., 305, 479-84 (1995)]. A 2.0 kb fragment containing the coding region of either human or murine Cox-1 or human or murine Cox-2 was cloned into a BamH1 site of the baculovirus transfer vector pVL1393 (Invitrogen) to generate the baculovirus transfer vectors for Cox-1 and Cox-2 in a manner similar to the method of D.R. O'Reilly et al (Baculovirus Expression Vectors: A Laboratory Manual (1992)). Recombinant baculoviruses were isolated by transfecting 4 μg of baculovirus transfer vector DNA into SF9 insect cells (2×108) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses were purified by three rounds of plaque purification and high titer (107-108 pfu/mL) stocks of virus were prepared. For large scale production, SF9 insect cells were infected in 10 liter fermentors (0.5×106/mL) with the recombinant baculovirus stock such that the multiplicity of infection was 0.1. After 72 hours the cells were centrifuged and the cell pellet homogenized in Tris/Sucrose (50 mM: 25%, pH 8.0) containing 1% 3-[(3-cholamidopropyl)dimethylammonio]-1-propanesulfonate (CHAPS). The homogenate was centrifuged at 10,000×G for 30 minutes, and the resultant supernatant was stored at −80° C. before being assayed for COX activity.

b. Assay for Cox-1 and Cox-2 Activity

Cox activity is assayed as PGE2 formed/μg protein/time using an ELISA to detect the prostaglandin released. CHAPS-solubilized insect cell membranes containing the appropriate Cox enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

c. Fast assay for Cox-1 and Cox-2 Activity

Cox activity was assayed as PGE2 formed/μg protein/time using an ELISA to detect the prostaglandin released. CHAPS-solubilized insect cell membranes containing the appropriate COX enzyme were incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds were pre-incubated with the enzyme for 10 minutes at 25° C. prior to the addition of arachidonic acid. Any reaction between the arachidonic acid and the enzyme was stopped after two minutes at 37° C./room temperature by transferring 40 μl of reaction mix into 160 μl ELISA buffer and 25 μM indomethacin. The PGE2 formed was measured by standard ELISA technology (Cayman Chemical). Results are shown below in Table 25.

TABLE 25

| | Cox-2* | Cox-1* |
|---|---|---|
| Example | IC₅₀ μM | IC₅₀ μM |
| 1 | 0.7 | 43 |
| 2 | >0.1 | 16.7 |
| 3 | <0.1 | 64.4 |
| 4 | <0.1 | 20.5 |
| 5 | <0.1 | 18.8 |
| 6 | <0.1 | 6.7 |
| 7 | 0.7 | >500 |
| 8 | <0.1 | 1.6 |
| 9 | 0.9 | 1.0 |
| 10 | <0.1 | 1.5 |
| 11 | <0.1 | 0.7 |
| 12 | 0.6 | >500 |
| 13 | 0.2 | >100 |
| 14 | 0.2 | 9.7 |
| 15 | 3.6 | 57 |
| 16 | <0.1 | 94.6 |
| 17 | <0.1 | 1.6 |
| 18 | <0.1 | 5.6 |
| 19 | <0.1 | 1.4 |
| 20 | <0.1 | 2.8 |
| 21 | 0.8 | >100 |
| 22 | 0.4 | >100 |
| 23 | <0.1 | 365 |
| 24 | <0.1 | 0.2 |

*fast assay

Biological Evaluation

A combination therapy of a Cox-2 selective inhibiting agent and a DNA topoisomerase I inhibiting agents for the treatment or prevention of a neoplasia disorder in a mammal can be evaluated as described in the following tests.

1. Lewis Lung Model:

Mice are injected subcutaneously in the left paw (1×106 tumor cells suspended in 30% Matrigel) and tumor volume is evaluated using a phlethysmometer twice a week for 30-60 days. Blood is drawn twice during the experiment in a 24 h protocol to assess plasma concentration and total exposure by AUC analysis. The data is expressed as the mean +/−SEM. Student's and Mann-Whitney tests is used to assess differences between means using the InStat software package. Celecoxib given in the diet at doses between 160-3200 ppm retards the growth of these tumors. The inhibitory effect of celecoxib is dose-dependent and ranges from 48% to 85% as compared with the control tumors. Analysis of lung metastasis is done in all the animals by counting metastasis in a stereomicroscope and by histochemical analysis of consecutive lung sections. Celecoxib does not affect lung metastasis at the lower dose of 160 ppm, however surface metastasis is reduced by more than 50% when given at doses between 480-3200 ppm. In addition, histopathological analysis revealed that celecoxib dose-dependently reduces the size of the metastasic lesions in the lung.

2. HT-29 Model:

Mice are injected subcutaneously in the left paw (1×10⁶ tumor cells suspended in 30% Matrigel) and tumor volume is evaluated using a phlethysmometer twice a week for 30-60 days. Implantation of human colon cancer cells (HT-29) into nude mice produces tumors that reach 0.6-2 ml between 30-50 days. Blood is drawn twice during the experiment in a 24 h protocol to assess plasma concentration and total exposure by AUC analysis. The data is expressed as the mean +/−SEM. Student's and Mann-Whitney tests is used to assess differences between means using the InStat software package.

Mice injected with HT-29 cancer cells are treated with a DNA topoisomerase I inhibiting agents i.p at doses of 50 mg/kg on days 5,7 and 9 in the presence or absence of celecoxib in the diet. The efficacy of both agents is determined by measuring tumor volume.

B. In a second assay, mice injected with HT-29 cancer cells are treated with a DNA topoisomerase I inhibiting agents on days 12 through 15. Mice injected with HT-29 cancer cells are treated with a DNA topoisomerase I inhibiting agents i.p at doses of 50 mg/kg on days 12, 13, 14, and 15 in the presence or absence of celecoxib in the diet. The efficacy of both agents is determined by measuring tumor volume.

C. In a third assay, mice injected with HT-29 colon cancer cells are treated with a DNA topoisomerase I inhibiting agents i.p 50 mg/kg on days 14 through 17 in the presence or absence of celecoxib (1600 ppm) and valdecoxib (160 ppm) in the diet. The efficacy of both agents is determined by measuring tumor volume.

3. NFSA Tumor Model:

The NFSA sarcoma is a nonimmunogenic and prostaglandin producing tumor that spontaneously developed in C3Hf/Kam mice. It exhibits an increased radioresponse if indomethacin is given prior to tumor irradiation. The NFSA tumor is relatively radioresistant and is strongly infiltrated by inflammatory mononuclear cells, primarily macrophages which secrete factors that stimulate tumor cell proliferation. Furthermore, this tumor produces a number of prostaglandins, including prostaglandin E₂ and prostaglandin I₂.

Solitary tumors are generated in the right hind legs of mice by the injection of 3×10⁵ viable NFSA tumor cells. Treatment with a Cox-2 selective inhibiting agent (6 mg/kg body weight) and a DNA topoisomerase I inhibiting agents or vehicle (0.05% Tween 20 and 0.95% polyethylene glycol) given in the drinking water is started when tumors are approximately 6 mm in diameter and the treatment ia continued for 10 consecutive days. Water bottles are changed every 3 days. In some experiments, tumor irradiation is performed 3-8 days after initiation of the treatment. The end points of the treatment are tumor growth delay (days) and TCD₅₀ (tumor control dose 50, defined as the radiation dose yielding local tumor cure in 50% of irradiated mice 120 days after irradiation). To obtain tumor growth curves, three mutually orthogonal diameters of tumors are measured daily with a vernier caliper, and the mean values are calculated.

Local tumor irradiation with single γ-ray doses of 30, 40, or 50 Gy is given when these tumors reach 8 mm in diameter. Irradiation to the tumor is delivered from a dual-source ¹³⁷CS irradiator at a dose rate of 6.31 Gy/minute. During irradiation, unanesthetized mice are immobilized on a jig and the tumor is centered in a circular radiation field 3 cm in diameter. Regression and regrowth of tumors is followed at 1-3 day intervals until the tumor diameter reaches approximately 14 mm.

The magnitude of tumor growth delay as a function of radiation dose with or without treatment with a Cox-2 selective inhibiting agent and a DNA topoisomerase I inhibiting agents is plotted to determine the enhancement of tumor response to radiation. This requires that tumor growth delay after radiation be expressed only as the absolute tumor growth delay, i.e., the time in days for tumors treated with radiation to grow from 8 to 12 mm in diameter minus the time in days for untreated tumors to reach the same size. It also requires that the effect of the combined a Cox-2 selective inhibiting agent and DNA topoisomerase I inhibiting agents plus-radiation treatment be expressed as the normalized tumor growth delay. Normalized tumor growth delay is defined as the time for tumors treated with both a Cox-2 selective inhibiting agent and radiation to grow from 8 to 12 mm in diameter minus the time in days for tumors treated with a Cox-2 selective inhibiting agent and DNA topoisomerase I inhibiting agents alone to reach the same size.

The contents of each of the references cited herein, including the contents of the references cited within these primary references, are herein incorporated by reference in their entirety.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the methods, combinations and compositions of the present invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for preventing or treating a neoplasia disorder in a subject that is in need of such prevention or treatment comprising administering to the subject a topoisomerase I inhibitor and a Cox-2 selective inhibitor, wherein the topoisomerase I inhibitor is edotecarin.

2. The method according to claim 1, wherein the Cox-2 selective inhibitor comprises at least one compound selected from the group consisting of celecoxib, parecoxib, deracoxib, valdecoxib, etoricoxib, meloxicam, rofecoxib, lumiracoxib, prodrugs of any of them, and mixtures thereof.

3. The method according to claim 1, wherein the Cox-2 selective inhibitor comprises at least one compound selected from the group consisting of celecoxib, parecoxib, valdecoxib, and mixtures thereof.

4. The method according to claim 1, wherein the Cox-2 selective inhibitor is celecoxib.

5. The method according to claim 1, wherein the Cox-2 selective inhibitor comprises a chromene Cox-2 selective inhibitor.

6. The method according to claim 5, wherein the chromene Cox-2 selective inhibitor comprises at least one compound selected from the group consisting of
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid. 6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid, (S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

7. The method according to claim 5, wherein the chromene Cox-2 selective inhibitor comprises at least one compound selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid, and
(2S)-6-chloro-5,7-dimethyl-2-(trifluoromethyl)-2H-chromene-3-carboxylic acid, and mixtures thereof.

8. The method according to claim 1, wherein the neoplasia disorder is at least one disorder selected from the group consisting of a neoplasia disorder of the lung, breast, skin, stomach, intestine, esophagus, bladder, head, neck, brain, cervix, and ovary.

9. The method according to claim 1, wherein the neoplasia disorder is at least one disorder selected from the group consisting of acral lentiginous melanoma, actinic keratosis, adenocarcinoma, adenoid cycstic carcinoma, adenoma, adenosarcoma, adenosquamous carcinoma, astrocytic tumor, bartholin gland carcinoma, basal cell carcinoma, bronchial gland carcinoma, capillary carcinoma, carcinoid, carcinoma, carcinosarcoma, cavernous carcinoma, cholangiocarcinoma, chondosarcoma, choriod plexus papilloma, choriod plexus carcinoma, clear cell carcinoma, cystadenoma, endodermal sinus tumor, endometrial hyperplasia, endometrial stromal sarcoma, endometrioid adenocarcinoma, ependymal carcinoma, epitheloid carcinoma, Ewing's sarcoma, fibrolamellar, focal nodular hyperplasia, gastrinoma, germ cell tumor, glioblastoma, glucagonoma, hemangiblastoma, hemangioendothelioma, hemangioma, hepatic adenoma, hepatic adenomatosis, hepatocellular carcinoma, insulinoma, interepithelial neoplasia, interepithelial squamous cell neoplasia, invasive squamous cell carcinoma, large cell carcinoma, leiomyosarcoma, a lentigo maligna melanoma, malignant melanoma, malignant mesothelial tumor, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillaryserous adenocarcinoma, pineal cell, pituitary tumor, plasmacytoma, pseudo sarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

10. A method for the prevention or treatment of topoisomerase I inhibitor-related diarrhea in a subject in need of such prevention or treatment wherein the method comprises administering to the subject a diarrhea preventing or treating-effective amount of a source of a Cox-2 inhibitor, thereby preventing or treating the topoisomerase I inhibitor-related diarrhea, wherein the topoisomerase I inhibitor is edotecarin.

11. A therapeutic composition comprising a topoisomerase I inhibitor and a Cox-2 selective inhibitor, wherein the topoisomerase I inhibitor is edotecarin.

12. The therapeutic composition according to claim 11, wherein the Cox-2 selective inhibitor comprises at least one compound selected from the group consisting of celecoxib, parecoxib, deracoxib, valdecoxib, etoricoxib, meloxicam, rofecoxib, lumiracoxib, prodrugs of any of them, and mixtures thereof.

13. The therapeutic composition according to claim 11, wherein the Cox-2 selective inhibitor is celecoxib.

14. The therapeutic composition according to claim 11, wherein the Cox-2 selective inhibitor comprises a chromene Cox-2 selective inhibitor.

15. The therapeutic composition according to claim 14, wherein the chromene Cox-2 selective inhibitor comprises at least one compound selected from the group consisting of
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid.
6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid,
(S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

16. The therapeutic composition according to claim 14, wherein the chromene Cox-2 selective inhibitor comprises at least one compound selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid,
(2S)-6-chloro-5,7-dimethyl-2-(trifluoromethyl)-2H-chromene-3-carboxylic acid, and mixtures thereof.

17. A pharmaceutical composition comprising a topoisomerase I inhibitor, a Cox-2 selective inhibitor, and a pharmaceutically acceptable carrier, wherein the topoisomerase I inhibitor is edotecarin.

18. The pharmaceutical composition according to claim 17, wherein the Cox-2 selective inhibitor at least one compound that is selected from the group consisting of celecoxib, parecoxib, deracoxib, valdecoxib, etoricoxib, meloxicam, rofecoxib, lumiracoxib, prodrugs of any of them, and mixtures thereof.

19. The pharmaceutical composition according to claim 17, wherein the Cox-2 selective inhibitor is celecoxib.

20. The pharmaceutical composition according to claim 17, wherein the Cox-2 selective inhibitor comprises a chromene Cox-2 selective inhibitor.

21. The pharmaceutical composition according to claim 20, wherein the chromene Cox-2 selective inhibitor comprises at least one compound selected from the group consisting of
6-chloro-2-trifluoromethyl-2H-1-benzopyran-3-carboxylic acid,
6-chloro-7-methyl-2-trifluoromethyl-2H-1-benzopyran-3-carboxylic acid,
8-(1-methylethyl)-2-trifluoromethyl-2H-1-benzopyran-3-carboxylic acid,
6-chloro-7-(1,1-dimethylethyl)-2-trifluoromethyl-2H-1-benzopyran-3-carboxylic acid,
6-chloro-8-(1-methylethyl)-2-trifluoromethyl-2H-1-benzopyran-3-carboxylic acid,
2-trifluoromethyl-3H-naphthopyran-3-carboxylic acid,
7-(1,1-dimethylethyl)-2-trifluoromethyl-2H-1-benzopyran-3-carboxylic acid,
6-bromo-2-trifluoromethyl-2H-1-benzopyran-3-carboxylic acid,
8-chloro-2-trifluoromethyl-2H-1-benzopyran-3-carboxylic acid,
6-trifluoromethoxy-2-trifluoromethyl-2H-1-benzopyran-3-carboxylic acid,
5,7-dichloro-2-trifluoromethyl-2H-1-benzopyran-3-carboxylic acid,
8-phenyl-2-trifluoromethyl-2H-1-benzopyran-3-carboxylic acid,
7,8-dimethyl-2-trifluoromethyl-2H-1-benzopyran-3-carboxylic acid,
6,8-bis(dimethylethyl)-2-trifluoromethyl-2H-1-benzopyran-3-carboxylic acid,
7-(1-methylethyl)-2-trifluoromethyl-2H-1-benzopyran-3-carboxylic acid,
7-phenyl-2-trifluoromethyl-2H-1-benzopyran-3-carboxylic acid,
6-chloro-7-ethyl-2-trifluoromethyl-2H-1-benzopyran-3-carboxylic acid,
6-chloro-8-ethyl-2-trifluoromethyl-2H-1-benzopyran-3-carboxylic acid,
6-chloro-7-phenyl-2-trifluoromethyl-2H-1-benzopyran-3-carboxylic acid,
6,7-dichloro-2-trifluoromethyl-2H-1-benzopyran-3-carboxylic acid,
6,8-dichloro-2-trifluoromethyl-2H-1-benzopyran-3-carboxylic acid,
2-trifluoromethyl-3H-naptho[2,1-b]pyran-3-carboxylic acid,
6-chloro-8-methyl-2-trifluoromethyl-2H-1-benzopyran-3-carboxylic acid,
8-chloro-6-methyl-2-trifluoromethyl-2H-1-benzopyran-3-carboxylic acid,
8-chloro-6-methoxy-2-trifluoromethyl-2H-1-benzopyran-3-carboxylic acid,
6-bromo-8-chloro-2-trifluoromethyl-2H-1-benzopyran-3-carboxylic acid,
8-bromo-6-fluoro-2-trifluoromethyl-2H-1-benzopyran-3-carboxylic acid,
8-bromo-6-methyl-2-trifluoromethyl-2H-1-benzopyran-3-carboxylic acid,
8-bromo-5-fluoro-2-trifluoromethyl-2H-1-benzopyran-3-carboxylic acid,
6-chloro-8-fluoro-2-trifluoromethyl-2H-1-benzopyran-3-carboxylic acid,
6-bromo-8-methoxy-2-trifluoromethyl-2H-1-benzopyran-3-carboxylic acid,
6-[[(phenylmethyl)amino]sulfonyl]-2-trifluoromethyl-2H-1-benzopyran-3-carboxylic acid,
6-[(dimethylamino)sulfonyl]-2-trifluoromethyl-2H-1-benzopyran-3-carboxylic acid,
6-[(methylamino)sulfonyl]-2-trifluoromethyl-2H-1-benzopyran-3-carboxylic acid,
6-[(4-morpholino)sulfonyl]-2-trifluoromethyl-2H-1-benzopyran-3-carboxylic acid,
6-[(1,1-dimethylethyl)aminosulfonyl]-2-trifluoromethyl-2H-1-benzopyran-3-carboxylic acid,
6-[(2-methylpropyl)aminosulfonyl]-2-trifluoromethyl-2H-1-benzopyran-3-carboxylic acid,
6-methylsulfonyl-2-trifluoromethyl-2H-1-benzopyran-3-carboxylic acid,
8-chloro-6-[[(phenylmethyl)amino]sulfonyl]-2-trifluoromethyl-2H-1-benzopyran-3-carboxylic acid,
6-phenylacetyl-2-trifluoromethyl-2H-1-benzopyran-3-carboxylic acid,
6,8-dibromo-2-trifluoromethyl-2H-1-benzopyran-3-carboxylic acid,
8-chloro-5,6-dimethyl-2-trifluoromethyl-2H-1-benzopyran-3-carboxylic acid,
6,8-dichloro-(S)-2-trifluoromethyl-2H-1-benzopyran-3-carboxylic acid,
6-benzylsulfonyl-2-trifluoromethyl-2H-1-benzopyran-3-carboxylic acid,
6-[[N-(2-furylmethyl)amino]sulfonyl]-2-trifluoromethyl-2H-1-benzopyran-3-carboxylic acid,
6-[[N-(2-phenylethyl)amino]sulfonyl]-2-trifluoromethyl-2H-1-benzopyran-3-carboxylic acid,
6-iodo-2-trifluoromethyl-2H-1-benzopyran-3-carboxylic acid,
7-(1,1-dimethylethyl)-2-pentafluoroethyl-2H-1-benzopyran-3-carboxylic acid,
6-chloro-2-trifluoromethyl-2H-1-benzothiopyran-3-carboxylic acid.
6-chloro-2-trifluoromethyl-2H-1-benzopyran-3-carboxylic acid,
(S)-6-chloro-2-trifluoromethyl-2H-1-benzopyran-3-carboxylic acid,
6-chloro-7-(1,1-dimethylethyl)-2-trifluoromethyl-2H-1-benzopyran-3-carboxylic acid,
(S)-6-chloro-7-(1,1-dimethylethyl)-2-(trifluoromethyl)-2H-1-benzopyran-3-carboxylic acid,
6-trifluoromethoxy-2-trifluoromethyl-2H-1-benzopyran-3-carboxylic acid,
(S)-6-trifluoromethoxy-2-trifluoromethyl-2H-1-benzopyran-3-carboxylic acid,
6-formyl-2-(trifluoromethyl)-2H-1-benzopyran-3-carboxylic acid,
6-(difluoromethyl)-2-(trifluoromethyl)-2H-1-benzopyran-3-carboxylic acid,
6,8-dichloro-7-methyl-2-(trifluoromethyl)-2H-1-benzopyran-3-carboxylic acid,
6,8-dichloro-2-trifluoromethyl-2H-1-benzopyran-3-carboxylic acid,
(S)-6,8-dichloro-2-(trifluoromethyl)-2H-1-benzopyran-3-carboxylic acid,
6-chloro-1,2-dihydro-2-(trifluoromethyl)-3-quinolinecarboxylic acid,
(S)-6-chloro-1,2-dihydro-2-(trifluoromethyl)-3-quinolinecarboxylic acid,
6,8-dichloro-1,2-dihydro-2-(trifluoromethyl)-3-quinolinecarboxylic acid,
7-(1,1-dimethylethyl)-2-trifluoromethyl-2H-1-benzopyran-3-carboxylic acid,
6,7-dichloro-2-trifluoromethyl-2H-1-benzopyran-3-carboxylic acid,
5,6-dichloro-2-(trifluoromethyl)-2H-1-benzopyran-3-carboxylic acid,
2,6-bis(trifluoromethyl)-2H-1-benzopyran-3-carboxylic acid,
5,6,7-trichloro-2-(trifluoromethyl)-2H-1-benzopyran-3-carboxylic acid,
6,7,8-trichloro-2-(trifluoromethyl)-2H-1-benzopyran-3-carboxylic acid,
6-iodo-1,2-dihydro-2-(trifluoromethyl)-3-quinolinecarboxylic acid,
6-bromo-1,2-dihydro-2-(trifluoromethyl)-3-quinolinecarboxylic acid,
6-chloro-7-methyl-2-(trifluoromethyl)-2H-1-benzothiopyran-3-carboxylic acid,
6,8-dichloro-2-trifluoromethyl-2H-1-benzothiopyran-3-carboxylic acid, and mixtures thereof.

22. The pharmaceutical composition according to claim 20, wherein the chromene Cox-2 selective inhibitor comprises at least one compound selected from the group consisting of
(S)-6-chloro-7-(1,1-dimethylethyl)-2-(trifluoromethyl)-2H-1-benzopyran-3-carboxylic acid,
(2S)-6,8-dimethyl-2-(trifluoromethyl)-2H-chromene-3-carboxylic acid,
(2S)-6-chloro-8-methyl-2-(trifluoromethyl)-2H-chromene-3-carboxylic acid,
(2S)-8-ethyl-6-(trifluoromethoxy)-2-(trifluoromethyl)-2H-chromene-3-carboxylic acid,
(S)-6,8-dichloro-2-(trifluoromethyl)-2H-1-benzopyran-3-carboxylic acid,
(2S)-6-chloro-5,7-dimethyl-2-(trifluoromethyl)-2H-chromene-3-carboxylic acid, and mixtures thereof.

23. A kit for preventing and treating neoplasia disorders in a subject that is in need of such prevention and treatment, the kit comprising one dosage form comprising a Cox-2 inhibitor and a second dosage form comprising a topoisomerase I inhibitor, wherein the topoisomerase I inhibitor is edotecarin.
